SEQUENCE LISTING (74 sequences)

SEQ ID NO: 1-2 - skipped (2 entries)

SEQ ID NO: 3 - DNA, 17 bp, Artificial Sequence
  Synthetic Oligonucleotide
accgtcagtgaaaacgt

SEQ ID NO: 4 - DNA, 17 bp, Artificial Sequence
  Synthetic Oligonucleotide
accgttagtgaaaacgt

SEQ ID NO: 5 - DNA, 17 bp, Artificial Sequence
  Synthetic Oligonucleotide
accatcagtgaaaacgt

SEQ ID NO: 6 - DNA, 17 bp, Artificial Sequence
  Synthetic Oligonucleotide
gccttgtagacgtaggg

SEQ ID NO: 7 - DNA, 17 bp, Artificial Sequence
  Synthetic Oligonucleotide
gccttgtaaacgtaggg

SEQ ID NO: 8 - DNA, 17 bp, Artificial Sequence
  Synthetic Oligonucleotide
gctttgtacacgtaagg

SEQ ID NO: 9 - DNA, 17 bp, Artificial Sequence
  Synthetic Oligonucleotide
gctttgtggacgtaggg

SEQ ID NO: 10 - DNA, 15 bp, Artificial Sequence
  Synthetic Oligonucleotide
tcactcgctggactc

SEQ ID NO: 11 - DNA, 15 bp, Artificial Sequence
  Synthetic Oligonucleotide
tcacgcgctggactc

SEQ ID NO: 12 - DNA, 15 bp, Artificial Sequence
  Synthetic Oligonucleotide
tcacacgttggactc

SEQ ID NO: 13 - DNA, 15 bp, Artificial Sequence
  Synthetic Oligonucleotide
tcacgcgctgaactc

SEQ ID NO: 14 - skipped

SEQ ID NO: 15 - DNA, 23 bp, Artificial Sequence
  Synthetic Oligonucleotide
cctgctctcacagatcacgggac

SEQ ID NO: 16 - DNA, 23 bp, Artificial Sequence
  Synthetic Oligonucleotide
cccgccctcacagatcacggaac

SEQ ID NO: 17 - DNA, 23 bp, Artificial Sequence
  Synthetic Oligonucleotide
cccgctctcacagatcacgggac

SEQ ID NO: 18 - DNA, 17 bp, Artificial Sequence
  Synthetic Oligonucleotide
accrtyagtgaaaacgt

SEQ ID NO: 19 - DNA, 23 bp, Artificial Sequence
  Synthetic Oligonucleotide
ccygcyctcacagatcacggrac

SEQ ID NO: 20 - DNA, 15 bp, Artificial Sequence
  Synthetic Oligonucleotide
tcacdcgytgractc

SEQ ID NO: 21 - DNA, 17 bp, Artificial Sequence
  Synthetic Oligonucleotide
gcyttgtrvacgtargg

SEQ ID NO: 22 - DNA, 128 bp, Artificial Sequence
  Amplification Product
accrtyagtgaaaacgttccygcyctyacagatcacggraccctgccgctgcgtagcagtatcagtggagtrcaycgdgtcaccatcactgacgcccgccgccggacctgtccrtacgtvracaaygc

SEQ ID NO: 23 - DNA, 110 bp, Artificial Sequence
  Amplification Product
ccygcyctyacagatcacggraccctgccgctgcgtagcagtatcagtggagtrcaycgdgtcaccatcactgacgcccgccgccggacctgtccrtacgtvracaaygc

SEQ ID NO: 24 - DNA, 82 bp, Artificial Sequence
  Amplification Product
accrtyagtgaaaacgttccygcyctyacagatcacggraccctgccgctgcgtagcagtatcagtggagtrcaycgdgtca

SEQ ID NO: 25 - DNA, 32767 bp, Adenovirus type 12
  ref <308>: DATABASE ACCESSION NUMBER: X73487.1
  ref <309>: DATABASE ENTRY DATE: 2008-10-23
  ref <313>: RELEVANT RESIDUES IN SEQ ID NO: (1)..(32767)
cctatctaataatataccttatactggactagtgccaatattaaaatgaagtgggcgtagtgtgtaatttgattgggtggaggtgtggctttggcgtgcttgtaagtttgggcggatgaggaagtggggcgcggcgtgggagccgggcgcgccggatgtgacgttttagacgccattttacacggaaatgatgttttttgggcgttgtttgtgcaaattttgtgttttaggcgcgaaaactgaaatgcggaagtgaaaattgatgacggcaattttattataggcgcggaatatttaccgagggcagagtgaactctgagcctctacgtgtgggtttcgatacgtgagcgacggggaaactccacgttggcgctcaaagggcgcgtttattgttctgtcagctgatcgtttgggtatttaatgccgccgtgttcgtcaagaggccactcttgagtgccagcgagaagagttttctctgccagctcattttcacggcgccattatgagaactgaaatgactcccttggtcctgtcgtatcaggaagctgacgacatattggagcatttggtggacaacttttttaacgaggtacccagtgatgatgatctttatgttccgtctctttacgaactgtatgatcttgatgtggagtctgccggtgaagataataatgaacaggcggtgaatgagttttttcccgaatcgcttattttagctgccagtgaggggttgtttttaccggagcctcctgtactttctcctgtctgtgagcctattgggggcgaatgtatgccacaactgcaccctgaagatatggatttattgtgctacgagatgggctttccctgtagcgattcggaagacgagcaagacgagaacggaatggcgcatgtttctgcatccgcagctgctgctgccgctgatagggaacgtgaggagtttcagttagaccatccagagttgcccggacacaattgtaagtcctgtgagcaccaccggaatagtactggaaatactgacttaatgtgctctttgtgctatctgcgagcctacaacatgttcatttacagtaagtgtgctatgggaggtgggaggtgatttttttttcttaagcagtgaaaaataatattttgttgtttttaggtcctgtttccgataatgagcctgaacctaatagcactttggatggcgatgagcgaccctcacccccgaaactaggaagtgcggttccagaaggagtaataaaacctgtgcctcagcgggtgactgggaggcgtagatgtgctgtggaaagcattttggatttgattcaagaggaagaaagagaacaaacagtgcctgttgatctgtcagtgaaacgccctagatgtaattaatggactttgagcacctgggcaataaaataggggtaatgtggtttttgtgagtcatgtataataaaactggtttcggttgaagtgtcttgttaatgtttgtttgggcgtggttaaacagggatataaagctgggttggtgttgctttgaatagttcatcttagtaatggagttggaaactgtgctgcaaagttttcagagcgttcgccagctcttgcagtatacctctaaaaacacttcaggtttttggaggtatctgtttggctctaccttaagcaaggtggtaaatagggtgaaagaagactatagagaggaatttgaaaacatattggccgactgtccagggcttttggcttcactagacctttgttaccacttggtgtttcaggaaaaagtggtcagatccttagatttttcatctgtgggacgaacggttgcttctattgcttttttggcaaccatattggataaatggagcgagaaatcccacctgagttgggattacatgctggattacatgtcaatgcagctgtggagggcatggctgaagaggagggtttgcatttactcgctggcgcggcctttgaccatgccgccgctgccgacgttgcaagaggagaaggaggaggagcggaaccctgcggtggtggagaagtaaacatggaacaacaggtgcaagaaggccatgtacttgactctggcgaagggcctagttgcgcagatgatagagataagcaggaaaaaaaagaaagtttaaaggaagctgctgttcttagtaggctaactgttaatctgatgtcccgcccgcgtttggaaactgtatattggcaggagttgcaggatgaatttcagcggggtgatatgcatttacagtacaaatacagttttgaacaattaaaaacccactggttagagccatgggaggatatggagtgtgctattaaagcttttgctaaattggccttacgtcctgattgtagctacagaattactaaaacagtaaccattacttcatgcgcctatattataggtaacggggcaatagttgaggtagatacaagcgacagagttgcttttagatgtcgaatgcagggtatgggcccaggggtggtgggtttggatggaattacatttataaatgttaggtttgctggagataagtttaaaggcattatgttcgaagctaatacctgtcttgtcttgcatggtgtttactttcttaactttagtaacatttgtgtagagtcttggaataaggtttctgctaggggctgtactttttatggatgttggaagggtttggtgggtagaccaaaaagtaaactgtctgtaaaaaagtgtttgtttgaaaaatgtgtacttgctttaattgtagagggggatgcacatattaggcataatgcagcttcagaaaatgcctgttttgtattattgaagggaatggctattttaaagcataatatggtttgtggggtgtctgatcaaactatgcgacgttttgttacctgtgctgatggaaattgtcataccttaaaaactgttcatattgtgagccacagtagacattgttggcctgtatgtgatcataacatgtttatgcgctgtaccatacatttaggcttaaggcggggtatgtttagaccttcccaatgtaacttcagccactcaaacattatgctggaacctgaagtgttttctagagtgtgtttaaatggggtatttgatttatctgtggaattatgtaaggttataagatataatgatgatactcgacatcgttgccgacagtgtgagtgtggtagcagtcatctagaacttcgtcccattgtgctaaatgtaactgaggagctgagaagtgaccaccttaccctgtcttgcctgcggactgactatgagtcaagtgatgaagacgacaactgaggtaagtgggtggagctaggtgggattataaaaggctggaagtcaactaaaaattgtttttgttcttttaacagcacgatgaacggaactactcagaacaacgctgcgctttttgatggaggggtttttagcccttatttgacttccaggttaccatattgggccggagtacgtcagaatgtggtaggatctacagtggacggtcgacctgtggcacctgcaaattcatcaacattaacctatgcaactattggaccctcgcctttggataccgccgccgccgctgcagcttccgcggccgcttctacggctcgcagtatggcagctgatttcagcttctacaatcacttggcttcgaatgctgtgacacgcaccgcagttcgagaggacattctgactgttatgcttgccaagcttgaaactctaactgctcagctggaagagctatcgcaaaaggttgaggaattagctgatgctactacccataccccagcccaacctgtaacccaataaagaaaaaacttaaattgagatggtgttatgaatctttattgatacttgttttttctgacatggtaagctcttgaccaccgttccctatcattaagaacacggtgaatgtgttccagtattttgtaaagatgagcctgtatattaaggtacattggcattaggccatctttgggatgaaggtaggaccattgaagggcttcatgttccgggttagtgttgtagataatccagtcatagcaacaacgctgggcatggtgattaaatatatcttttaacaacaagctaattgctaatggaagacctttagtataggtattgataaaacggttaagctgggtgggatgcatccgaggtgacatgatatgaagttttgattgtattttgagattggcaatgttacctgccaaatctcttcttggattcatattgtggagaaccacgaaaacggtgtagccagtacacttgggaaatttgtcatggagtttagaaggaaaggcatggaaaaacttggaaacgcctttgtgacttcccaaattttccatacactcatccattattatggcaattggaccgcgagcagcggcttgagcaaaaatgttttctggatcagaaacatcatagttgtggtctagagttaggtcatcgtaggacaacttaacaaatttaggacacagcgttccagattgtggaataatagttccctctggtcctgggacataatttccctcacaaatttgcatttcccaagatttaatttcagatgggggaatcatgtccacttgcggaacaataaaaaaaacagtttctggagcaggtgtaaccagctgggcagaaagcaaattacgcaacaactgagacttcccacagccagtgggtccataaattaccccaattacaggttgcaagtgatagtttaacgaggtgcagctgccgtcttcgtggagaagcggagccacttcattcatcatttgtcggacgcggatgttttgcttggccagttcccctaacagacgctctccgcctaaggaaagtaactcttgtaaagatttgaaatttttaagtggctttaggccatcggccataggcatgtggtccagggtttgcttcagcagttgcaagcgatcccatagctcagttatattttctatgccatctcgatccagcaaacttcctcgttgcgggggtttggctggctgttgctgtaaggaacgaggcggtgagcatccaaatggacgagggttttgtccttccagggacgtaatgtgcgcgtcagggttgtttcggtcacggtgaatggatgcgctcctggttgagcgctggccagtgtgcgctttaaactgaggcggctggtgctgaagcgcgtgtcttctccctgtgcttcggcaaggtagcattttaacataagatcataagacaaagcctctgtagcgtggcctttagcccgtatttttcctttggaggtgctcccgcagtgaggacactgaaggcatttaagggcgtacagttttggagccaaaaaaacagattctggagaataagcatctgcgccacaataactacaaacagtttcacattcaactgaccaggtcagctcaggacatgatggatcaaaaacaagtttccctccgtactttttgatgcgtttcttaccttgcgactccataaggcggcgtcctttctctgtgacaaaaagactgtcagtgtctccgtatacagatttaaggggtctatccttcagtggtattccgcggtcctcctcgtacaggaattctgaccactctgacacaaaagctctagtccaagcaagtacaaaggaagccacatgggaagggtaccgatcgttgttaattaaagggttagaactttctaaggtgtgtaaacacatgtctccttcttcagcgtccatgaatgtgattggtttgtaggtgtaagtcacgtgttcacaattttctggtggtgggctataaaaaggggcgggtccttggtcttcatcgctttcttctgcttcgctgtttacgagcgccaactggttgggtgagtacacgcgctcaaaggcaggcattacctctgtactcaacgtgtcagtttctataaacgatgaggatttgatgtttaatcgccccgctgcaatttctttcattaggctttcttccatttgatcagaaaaaactatttttttgttatctagtttggtagcaaaagatccgtacaaggcattggaaagcagcttggctatagatcttagggtttgatttttgtccctatcggcccgttcttttgcggcaatattgagttgcacatattcgcgtgccaggcatttccaggtggggaaaatggtggtgcgctcgtcagatagcaagcgtaagcgccacccgcgattatgcagtgtaaccagatctacgctggtaactacttcaccgcgcaagctttcattggtccaggctaaacgaccgccttttctagaacaaaaaggaggaagaacatccaactgattttcatctggggggtcggcatctatagtaaaaatgccaggacaaagatttttgtcaaaataatcaattttgcaagtgtaattttccagcgccacctgccattgccgcacggccaatgcccgctcataggggttaaggggaggaccccaaggcatggggtgtgtgagggccgatgcatacatgccgcaaatatcatatacatatatgggctcttttagtactcctatgtaagtaggatagcacctgccgccacgaatgctggcgcgaacgtagtcatatagctcatgtgaaggcgccaggatgttgggcccaagatgtgtgcgctgtggtttttcggcgcggtacaaaatttgtctgaaaattgcatgagagttagaggaaatggtaggacgctgaaacacattaaaatgtgccgcgtcaagacccactgcgtcagtaacaaactgggcgtatgagctacgcagtttttctaccaatgaggcagtcacaagtacatccagggcacaatagtttaatgtttccccgataagattgtaatttttttctcctttttttttccatagttcttgatttaggaggtattcctccttatccttccagtactcctccaggggaaacccatttgcatctgcacggtaagaaccaagcatataaaactgatttaccgccttgtacggacaacatcctttttctacaggcagggcatacgcttgtgcagcctttcttaaagatgtatgagtaagagcaaaggtatctctgaccattacttttaaatactggtatttaaaatcttggtcgtcacaccctccgtgttcccacagtaggaagttagttcgctttttgtagtggggattgggaagggcaaaagtaatatcattaaataatattttgccagctcttggaataaaatttctagaaattttaaagggtccagggacgtccaagcggttattgattacctgagcggcaagaacaatttcatcaaatccattaatattgtgtcctactatatacaactctacaaatcttggctcacccttaattgcaggggctcttttaagatcttcgtaggaaagatcttcaagcgcgactagtccgttttcttcttgagcccattgagacaagtgtggatttttttgtaaaaaagtcatccaaagatcagtagctaaggaggtttgtaagcggtttctataggtacgaaactgttgaccgaccttcattttttctggggttaagcagtagaaagtagtagagtctttttcccattggtcccatccaagttctaatgcaagttgtaaggcatgtttgacaagattgtcatccccagacagtttcatcaccagcataaatgggacaagttgctttccaaatgcccccatccaggtgtaggtttctacatcataggtaataaaaaggcgctcagtgcgaggatgcgaaccgattgggaaaaagtggatctcctgccaccagttggaagaatggctgttgatgtgatgaaagtagaaatctcgtcggcggacagagcattcatgctgatgtttgtaaaagcgtgcgcagtgttcgcatcgttgcacgggctgtatctgttgaatgaggtgtacctggcggcctcgcaccagaaagcagatgggaaaatcaataccacttggcagctgccgttcgtcctcttcctcttctgctgcattgccactaccgtttggatcctcgaaagcgagaacggagagggtgacggtgcccctcgacctgcatgtccagatttcagcacgagaggggcggaaacgggaaatcagggcgtacagcctggagctgtccatggtatcagtcagagagaaaagcatgtccgcggggacagcgcgcaagttgacttcgcacaggcgggtaagagcaggctggaggtgcaggtaatacttaatttctagaggcgtgccgttggcagagtctattgcgtgaagtattccatgagcccggggactaaccacggttccacggtgcacttttccaatgcgcctgcttaaaatcggcggcgcggacgagctcccggaggaagcggcggttcgggtcctgcgggaagcgggggaagcggtatgtcggcctgacgctctggcaggggaaggtgttgagcccgaagttgactggcatgggcgactacccggcgattgatatcttgaatctgtcggcgttgtgtaaacactaccggccctgttgttttgaacctgaaagaaagttcaacagaatcaatctcagtgtcatttactgcagcctgtcttaaaatctcctgaacgtcgcctgagttatcttggtaggcaatttctgccattaattgatcaatttcttcctcctggaggtctccatgtcccgcacgttcaatagtggctgcaaggtcattagatatccgactcataagctgtgaaaatgcgtttagtccaatttcgttccagactcggctgtatactacccctccttcgctgtcccgagcgcgcataaccacttgcgccaagttgagttccacgagccgtgcgaacacgccgtagttgcgcaagcgctgaaacaggtagtttaaggtggtggcaacgtgttctgagacgaagaaatacagaatccaccgacgaagcgtcagctcgttgatgtcacctaaggcttcaagacgttccatggcttcgtaaaagtctactgcaaaattgaaaaactgggagttgcgagctgccaccgtcaattcttcttccaacagacgaataagctcggccaccgtctcgcgcacttcttgctgaaatgcgcccggaactatttcttgttcttcctcttctacctccattatttcttcctcgaccacaggtggtgggggttgtcttcttcgacgccggcgaacgggcagcctgtctacaaatctttcaatcatttcgccgcgacggcggcgcatagtttcggttactgctcgaccgttttcacgtggtcgtaactcaaaaactccacctctaagttctgtttcatgtaaaatgggaaatgaggcgttgcgaggggcgttaggtagggatacagcgctgattatgcattttattatttgctgcgtaggaactccgcgcaaggagctaagcgtctgcatatccaccgggtcggagaacctttcaagaaaggcatctagccagtcacagtcacaaggtaggctaagttttgtttcttctaaagtaccaggaagctgagcaatgctactaataatgtaattgaagtaagctgttttaagcccacgaatggttttaagaagcaccacatctttgggtccggcttgttgaattcgcaggcggtctgccattccccacacgtcactttgacatcgtccaagatctttgtagtagtcttgcattaacctttccacctctacctcgcggtttccgcgatcagccatgtgcgtgcttccgtagccttgcagcggttgtaataaagctaaatctgccactacccgttccgcaagcactgcctgttgaatttgggtaagggtggttgcaaagtcatccacatctacaaagcggtgataagctcctgcattaatggtgtagctgcagtttgtcattactgaccaattaacagtttgcgtgcctggctgtacagtttctgtgtatcgcaagcgtgagtaagcccgagagtcaaaaacatagtcattgcaggtgcgcactaggtattgatagcccacaaggaaatgaggaggaggttcgcgatacaacggccagccaagcgtagccgcagcacctggagcgagatcttccaacatgaggcggtggtattcatatatgtatctggacatccatgtgatgccggcagcggtagttgttgctcgcataaattcgcgggctcggttccaaatattgcgcaggggtaaaaagcgttcaatagttgccacgctttgaccggtcaggcgtgcgcagtcttgaatgctctggacatggaaaaaatgaaagttggtaagcgactcccttccgtggtttggtggaaaagtcacaagggtaccatagcgaggaaccccggttcgaaaccggcaggatccgctatgagcacaagtgaggcgcttgcgcgttgaacccggccaaggacccccagacacggagaggagtctttttttatttattttttcttagatgcatcctgtcctgcgacaaatgcgacctcagcccagggcaaccacggcctcagcagcggtggcgctttcgggctctggcgaacaggaagagcctcaatgtcctacattggagttggaagaaggagaaggcatagcccgattgggcgcccactctcctgagcgtcacccaagggtgcagctcgcccgggacagtcgcgtggcatttgtgcctcgtcagaacatgtttcgcgacaacagcggggaggaagctgaggaaatgcgagactgcaggtttagggccggtcgcgagctgcgccgcggatttaatcgcgagcgactgctgcgtgaggaggactttgagccagatgaacattcggggattagttctgcacgggcccatgtatcagcagccaacttagtaacagcatatgaacaaacggttacagaggaacgtaactttcaaaaaagctttaataaccatgtgcgcacactaatagcgcgagaagaagtagccattggtttaatgcatctttgggactttgtagaagcttatgtacataatccagcaagtaaacccctaactgcccagctgttcttaatagttcaacatagtagagacaatgaaacttttagggatgcaatgcttaacatagctgaaccccagggtcggtggttactcgatttaattaacattctgcagagcattgtggttcaggaacgcagtcttagtttggcagacaaggtggccgccattaattactccatgttaagtttgggaaagttttatgctcgtaaaatctacaaaagtccgtatgttcccattgacaaggaagtgaagatagacagcttttatatgcgcatggctttaaaggtactaacattaagcgacgatcttggagtgtaccgcaatgaccgaatccacaaagcagtaagcgccagtcgccgcagagagctaagcgacaaagagcttatgcatagcttacaaagggcgctgacgggagcaggaacagaggacgagtcgttctttgatatgggcgcagacctacggtggcagccaagcgctcgcgctttggaggcagctggagtggcgtctgctgacgtcactggcgatgacgatgacgaagaccagtacgaggactgatcggccgtaccttttgttagatgcagcgaccggcgatcatcgcggagagggctcctaacctggatcccgcggttttggcggccatgcaaagccagccttctggcgttacagcttcagatgactggacagcggccatggatcgtattatggctttaacggcgcgcagtcctgatgcttttcgccagcagccccaagctaaccgcttttcggccattttggaagcagtagtgccgtctcgtactaaccctactcacgagaaagtgttaaccattgtaaatgctttgttggatagcaaagccatccgcaaagatgaggctggtttaatatacaacgctttgcttgagcgcgtggcacgctataacagtaccaatgtgcaggctaacttagaccggatgggtacagatgtaaaggaggcgctggctcaacgagagcgctttcatcgcgatggtaatcttggttcgctagtagcattaaacgcttttttgagtactcagccggctaatgttccgcgtggtcaggaagattatacaaacttcatcagcgccttgcgactaatggttactgaagtgcctcaaagtgaagtgtatcagtctggacccgattacttttttcaaacgtccaggcagggtttgcaaaccgtaaacttaactcaggcttttaaaaatttgcaaggtttgtggggggttcgtgctccagtaggcgatcgttcaactttgtccagtttactaacaccaaactcgcgcctattactgttgctaattgccccctttaccaacaccaacagtttaagtcgagattcatacctgggtcacttagttactttgtaccgcgaagccattggtcaagcgcaggtagacgaacaaacttatcaagaaataaccagtgttagtcgcgcactgggccaggaggacactggcagtttagaggccacacttaactttttactaactaaccgtcgccagcaagtgcctcctcagtacactttaaatgcggaagaagaacgcatattgcgctatgtacagcaatctgtaagtttgtatcttatgcgtgagggtgccacccccagtgccgccttagacatgacagcgcgcaatatggagccgtccttctacgcttccaatcgagctttcattaatcgcttgatggattaccttcaccgcgctgcggccatgaacggggaatactttacaaatgcaattctaaatccgcattggttgccccctcctggattttacactggtgaatttgatttgccggaaggaaatgatggctttttgtgggatgatgttacggacagtctgtttagtcctgcagttattggacaccatggtaaaaaggaagcaggtgatgaaggtcccttgcttgactctcgggcgagttctccattccccagtttaactagtttacccgccagtgttaacagcggtcgtaccaccagaccccgactaacaggtgaaagtgaatacttaaatgaccccatcttgtttccagtgcgcgacaaaaattttcccaacaatggcatagaaagtttggtagataaaatgtctcgctggaaaacatatgcacaagagcggcgagaatgggaggaaagacagccaagaccagttcgccctcctaggcaacgttggcagcgacgcaaaaaaggggcacatgcgggggatgaaggaagcgatgactcagctgacgacagtagtgtattagatttaggagggtcaggaaacccatttgctcatttgcgcccacagggttgcatagggtcattgtattaaattgaataaaagcatacttaccaaagccatggcgaccagtgttcgtcttattttccttcttccgttagctgtgaaatgaggcgcgcggtggaactgcagacagtggcttttcctgagacaccacctccctcttacgaaaccgtgatggcagcggcgccaccctacgtgcctccccgctatttgggtcctacggagggaagaaacagtatccgttactcggaattgtcaccgttgtacgataccactcgagtgtacttggtggacaacaagtcttctgacattgcttcattgaattaccagaatgatcacagcaactttttaaccactgtagtgcaaaataatgactattcccctatagaggctggcacgcaaactattaactttgatgaaaggtctagatggggtggagatttaaaaaccatcttacataccaacatgccaaacgtgaacgattttatgtttaccaccaaatttaaggccagggtaatggtggctaggaaaacaaacaacgaaggccaaaccattttagaatatgagtgggcagaatttgtgctacccgagggtaactattcggaaaccatgactattgacttaatgaacaatgctattattgagcattatttgcgagtaggaagacagcatggagtgctggaaagtgacattggagttaagtttgacaccagaaactttcgtctgggttgggaccccgaaacccaattagtaactccgggagtgtacactaatgaggcttttcatccagatatagtactgcttccaggttgcggggttgattttacagagagcagattaagcaacatactaggtataagaaagaggcagccgtttcaggaaggatttgtgattatgtatgaacacttagagggaggcaatattccagctcttttggatgtaaaaaaatacgaaaacagtctgcaggatcaaaacactgtaagaggagacaactttattgccttaaataaggctgctaggattgaaccggttgaaacagaccccaaaggacgcagttacaacttgcttccagacaaaaaaaatactaaatatcgcagctggtatttggcatacaactacggagacccagaaaaaggagttcggtcatggactctactaacaactccagatgtaacaggcggctccgaacaggtgtactggtccctacccgatatgatgcaagatccggtgacttttcgctcctcgcgtcaagttagcaactatcctgtagttgcagcagaattactgccagttcatgctaaaagcttctacaacgagcaagccgtctactcacagcttattcgccagtcaaccgcgcttacgcgcgtgtttaatcgctttcccgagaaccagatactggtgcgtccaccagccgctaccatcactaccgtcagtgaaaacgttcccgcccttacagatcacgggaccctgccgctgcgtagcagtatcagtggagttcagcgagtcaccatcactgacgcccgccgccggacctgtccctacgtttacaaagcactgggcatagtttctccacgagtgctttctagtcgcactttttaaaaaagtgtggtaacatgtccattttggtttcgccaagtaacaacacgggctggggactgggtgccgcccgcatgtatggaggagctaaaacaaggtctagccaacatccagtgcgcgtacgcggacattaccgagctccatggggcgcgcatacccgaggacgcactggtcgcaccactgtagacgatgttattgactcggtagtggccgatgctcgcaagtaccgcgcgcccgctgaaacagcagggtctactgttgatgcagtaattgatgaggtagtggcaaacgcgcgggcttatgcaaggcgccgcagacggctgcgtcgccggcgtagaccaaccaccgccatgcgcgcggccagagcgttggttcgacgggccaggcgcattgggcggcgagctatgatgcgggcagccaggcgggctgcaacgcctgccggtcgagcgcggagacgggccgcagctgcggccgcaacagctattgcaaacctagctgctccgcgacgaggaaatgtatactgggtgcgcgactcagtgaccgggacgcgtgtgccagttcgtacgcgtccacctcacccttagaagacaaagagtgactcaatgtctgttatgtatgcccagcatgaccaaacgcaagttcaaagaagagctgctgcaggccttagcgcctgaaatatatggcccatcggataaccttaccaagcgcgatatcaagcatgttaaaaaacgggaaaaaaaagaggaagaagtcgccgcggcgtcagcagacggcgtcgagtttgtgcgctcatttgcgcccagacgtagggtacagtggaagggacggcaagtaaaacgcattttgcgaccgggcaccacagtggttttttctcccggagagcgaacgattatgcgtcccctaaagcgcgagtacgacgaagtgtacgcagacgatgacattttggagcaagcggcacaacagactggggaatttgcatatggaaaaaaagggcgttacggagacaaaattgctattcctttggacgagggaaatccaacacccagtttaaaggctgtcactttgcaacaagtgttgcccgtccttgggccttcggaagaaaagcgtggaattaaaagggaagccatggatgaattgcagcctacaatgcaactgatggtgcctaagcggcaaaagttagaggacgtactagagcacatgaaggtggatcctagcgtacagccagatgtaaaagtacgtccgataaaaaaggtagctccaggattgggagttcaaacagtggacattcaaattcctgtgcaaactgcattgggtgaaactatggaaatccaaacttcgccaataaaaacaacggtgaacgcaagcgtgcaaacagacccttggtacccgccagtgctttcaacaaaaaaaaagcgtcactacagacaaacaagttcgcttttgccagactacgttttacatccttccattgtgcccacgcctgggtaccgtgggacaacttttcagcgccgagccacagcccctagccgtagacgaggtccatcacgccgtagacgtcgacgcaaagccactttagccccagcggcagtacgtcgcgttgtacaaagggggcgcacactaatacttccatccgtgcgttaccaccctagcattctctaacaagctgcgctgccgttttttcagatggctcttacttgccgaatgcgcatacccattccaggatacagaggacgaccccgccggaggaaagggctgaccgggaacggtcgatttcggcggcgtagtatgcgcagacgcatgaagggtggggtgctgcccttcctaattccacttattgctgcggccattggagccgttcccggaattgcctcagtagccttgcaggcttctcgaaaaaattaaaataaaataaaacttccaacttattactggtactatgactgttttatgcagactaaatggaagacatcaatttttcgtcgctggccccgcgacacggcacgcggccgtacatgggcacctggaacgagatcggcacgagccagctgaacgggggcgccttcaattggaacagtatctggagcggtcttaaaaattttggttccacgattaagacatatggcaccaaggcgtggaacagccaaaccggccagatgctaagggacaagttaaaagaccaaaattttcaacagaaagttgtagatggtctggcttcgggaattaatggagttgtagacatagccaatcaggctgtacagaaaaaaattgccaaccgtttagagccgcggcccgacgaggtaatggtagaggaaaagctgccacctctagaaactgtgcccggatccgttccaaccaaaggagaaaagcggccacggccggatgcagaggaaaccttagtaacgcacacaacagaaccgccgtcctatgaggaagcaataaaacaaggagccgctctgtcacctaccacctatcccatgaccaagcctattttacccatggctactagagtgtatggaaaaaacgaaaatgtgcctatgacccttgagctgcctcctttgccagaacccactatcgcggatcccgtaggttccgttcctgttgcatctgttccagttgcatcgacagtgagccgtccagcagtgcggcctgttgccgtggctagcttgcgaaacccacgatccagtaattggcaaagtaccctaaacagtattgtgggactgggagtaaagtctctcaaacgccgacgctgctactaacattaaaagacgagtgttaattcccatctgtgtatacgcctcctatgttagcgccagaggaccaacgcgtgaatcgcagtcaccaccagcgctttcaagatggccactccctcgatgatgccgcagtggtcttacatgcacatcgccggtcaggatgcctcggagtacctgagtcccggtctggtgcaattcgcccgcgccacggacacctacttcaccctgggaaacaagtttagaaaccccaccgtggctcccacccatgatgttaccaccgatcgctcgcagcgtctgacgctgcgttttgtgcccgtggatcgggaagatactacctactcctacaaggctcgctttacgctggctgtgggtgacaaccgcgtgttagacatggctagttcttactttgacattcgaggggtactggatcgtggtcccagttttaagccctattccggaaccgcctacaattctttggcaccaaaaggcgctcctaatgcttcacaatggtcagataacgctaagcttaatacctttgctcaggcgccgtatcttagcgacactatcaccgccgccgatggtattaaagttggaacagacaccgcccaggcaggcgcggcggtgtatgccaacaaaacttatcagccagagccgcaagtaggaccaagtgaatggaacaccagcattgaaaacgttaaagctggcgggagggcattaaagcaaaccactgcaatgcagccgtgctatggctcctacgctcgtccaaccaacgaacacggaggacaatccaaggatgacaacattgaacttaagttctttgattcagctaacaatgcagcaaacactgctcaagttgtgttctataccgaagacgtaaaccttgaaatgccagacacgcatcttgtgtttaagcctactgttaccaatggaacaattgcttctgagtcgctgttgggacagcaagcagcgccaaatagagcaaactacattgcattcagagataattttattggcctgatgtattacaacagtacaggcaacatgggtgtattggccgggcaagcttcccaacttaacgcagtagtagacctgcaagacagaaatacagagctgtcataccagttaatgctggatgctttgggagacagaacacggtacttttccttgtggaattccgcagtggacagttacgaccctgacgttcgcgttattgagaatcacggggtagaggatgaactaccaaattattgctttcctcttagcgcagtaggtgaaataaaaaattacaaaggcattaagccagataacggaggaggaggtggctggactgccgacaacactgtcagtgaagcaaaccacataggcattgggaatatagccgccatggaaattaatttgcaggctaatttgtggagaagcttcttgtactcaaatgtgggcttatacctaccagacgacttaaaatacactccaggaaacataaaactacctgataacaagaacacctacgagtacatgaacgggcgtgtgactgccccggggttggtggatacctatgtcaatatcggcgctcgctggtccccagatgtgatggataatgtaaacccttttaaccaccaccgaaacgcagggttgcgctacagatccatgttgctaggcaatgggagatttgttccttttcacattcaggtgccgcaaaaattttttgccatcagaaatttgttgctgttgcccggttcctacacttacgaatggaactttagaaaggatgtaaacatgattcttcagagcacactgggaaatgatcttcgggtggacggagccagcgttcgctttgacaacattgccctgtatgctaacttttttcccatggcacataacacagcttctactttagaagccatgttaagaaatgacaccaacgaccagtcttttaacgattatttgtgtgctgcaaacatgctgtatcccatcccagctaacgccaccagcgtgcccatttcaataccttcgcgaaattgggcggcatttagaggctggagctttactcgcctaaaaactaaagaaactccttccctgggttcagggtttgacccctactttgtatactctggaaccattccctatttagacggcaccttttacctaaaccacacttttaagaaggtgtcaatcatgtttgactcctccgtgagttggcctggaaatgaccgtttgctaaccccaaatgaatttgaaataaagcgttctgtggatggggagggatacaatgtggcccaatgcaatatgactaaggattggttcctaatacaaatgcttagtcattacaacattggataccaaggtttttacattccagagagctacaaggaccgcatgtattctttctttagaaactttcagcccatgagtaggcaagttgtggataccacagaatataagaactacaaaaaagtaaccgtagagtttcaacataacaactcaggattcgtgggatacctgggccccactatgcgggagggacaagcttaccccgccaactatccctaccctcttataggccaaacagctgtggaaagcatcacacagaaaaagtttctatgcgatcgtgttatgtggcgcatcccattttctagtaacttcatgtctatgggggcgctaacggatcttgggcaaaatatgctgtacgcaaactcagcccatgctctagacatgacatttgaggtggatccaatggatgagcctacccttctttatgttttatttgaagttttcgacgtggtacgcattcaccagccacaccgcggcgtcattgaagcggtctacctgcgcacgcccttctcggcgggtaacgctaccacctaagaaggcaccctcccagactgctgtaatgggttcaagcgaacaggagctgacggccattgttcgagatctaggctgtggaccctattttttgggaacctttgacaaacgttttccgggttttgtgtctcgcgaccgcttatcatgtgctattgttaacactgccggtcgcgaaactgggggcgtacactggctggcttttggatggaaccccaaatcgcacacttgctatttattcgatccatttggattttctgatcaacgactaaaacaaatctatcagtttgagtacgaaagtctgttgcgccgtagtgcgctagcggccactaaagaccgatgcgttaccctagaaaagtcaacccaaactgtacaaggaccgttttctgcagcgtgcggcctgttttgttgtatgttcttacacgcttttactcactggcctgaccatccaatggataaaaatcccactatggacctacttactggggtgcctaattgtatgctacaaagtcctcaggtagtgggcacattgcaacgcaatcagaatgaattgtataaattcttaaacaatctgtccccttactttcgtcacaaccgcgagcgcatagaaaaagctacatcttttactaaaatgcaaaatggactcaaataaacgtgtacacaatgcattaataataaaaccattttattagctcattggagtacaagcttgactgttttattaaaaatcaaatggctcttcgcgacagtcgccgtggttggtgggcagggatatgtttctgtactgcaaacgctgatgccacttgaattctggaataacaagcctagggggggagccgtcaaaattttctccccacagctggcgcacaagttgcagggcgcccataacatcaggagcagaaatcttgaagtcgcaattagggccagcattgccgcgcgcattgcgataaactggatttgcgcactgaaaaaccaacaaacacggatacttaatactggctaacgctccagggtcggttacttcgttgatatcaatgttatccacattgctgaggttaaaaggagtgattttacacagttgacgccccatccgtggcaggccatcttgcttgtttaaacattcgcagcgcactggcataaggagacgtttttgcccatgtcgcatgtgagggtagtcggccagcataaaagcttcaatttgcctaaaagctatttgagccttcattccttcagaataaaacaagccgcaggactttccggagaaagaattattcccgcagccaacatcatgaaaacagcagcgggcatcgtcgtttttaatttgaactacattacgcccccagcggttttgcgccaccttggctttcgaggggttctctttcaacgctcgttgcccactttcgctggttacatccatttccaccaaatgctctttgcgcaccatctccattccatgcaggcatctaagctccccttcgcgctcggtacacttatgctcccacacgcagcaaccggtgggttcccaggaattctgttggacaccggcataagcttgcatatatccttgcaaaaagcgtcccatgagctcctgaaaggttttttgggatgaaaaagtcagctgcaaaccgcgcttttcttcgttgagccatgttgtgcatattttcttgtacacgctgccctgatccggcaaaaaacgaaaggtggcgcgctcgtcgtgatccacatggtacttttccattagcatagccatggcttccatgcctttttcccaagctgaaactaggggctggcttgccggattgcgaacaacaacaacattcttttcattttcgtcgctgttttgagcggaagccttcaaaacgtgtacctgcctggtttccattttttgaaaagactgagaaccgtctgcatgatgcataatgcggacgggcggcatgctgaaacccattactcctaaaactgctcttggtggttctgcctcttcttcttctgcactctctggggaaagaggtatcgcagccatagatttcttgacttttttctttggaggtaaaggcacagcttccagttcttcttcgctttcggaatccagaaagtatctgcccatttttggcggcggcggctgagcgctgcggtctggggtgcgctccctctgtgagtgctgattgctggccattatttaatcctaggcaaagaaacacatgatggatctggagccacaggaaagcttaaccgcccccaccgctcccgccattggcgctacggctgtcatggagaaggacaaaagtctactcataccccaagacgcaccggttgagcagaacttgggctacgagactccccccgaggaatttgaaggctttcttcaaatccaaaagcaaccaaatgagcaaaacgctgggctcgaggaccatgactacctaaacgagggagatgtcctgtttaaacatctacagcgacaaagcactatcgttcgcgacgccatatctgatcgctcttcaataccagtttcaattgcagaactatcttgcatctacgaacgcaacctgttctccccacgtgtgccccctaaacggcaagccaacggcacatgcgagccaaatcctcgccttaacttctacccagtttttgcagtgccagaagcactggcaacataccatattttctttaaaaatcacaaaatacccctatcctgtcgagctaaccgcagccgcgcagatgagcttcttgctttaagggctggcgcttccatacctgggattgtgtccttggaagaggtgcctaaaatttttgaaggtttaggtcgggatgaaaaacgagcagcaaatgccctgcaaaaagaaaatgaacaaaatcaccatgggaatagtgctctaatagaactggaaggtgacaatgcccgcctggcagttttaaagcgcaatattgaggttactcactttgcctacccggcagtaaatcttccgccaaaggtaatgagcgcagtgatgaatcagctactaattaagcgagcccaacccattgacaaagatgcaaacttgcaagacccggaggcaacagatgatggaaagccggttgtaagcgacgagcaattaactaagtggttgggaacagacaattccaacgaactacaacagcggcgtaaactcatgatggccgccgtacttgtaactgtggaactcgagtgcatgcatcgttttttctccgacatcaccacattgcgcaaaattgaggaatgtcttcactacactttccgccatggctacgtgcgccaagcctgtaaaatttctaatgtggagctgagcaatctagtttcttacatgggcatcttgcatgaaaaccgattgggacagaacgtgctacactcaacactacgcgatgaagcacgcagagattacgtgcgagactgcatttaccttttcctgttacatacctggcaaactgggatgggtgtttggcagcaatgcttggaagaaaaaaaccttcgagaactaaacaaactgttagacagagcactaaaatccctatggaccggttttgacgaacggacagtagctgcagagctagctgacataattttcccagaaaggttaatgataaccttgcaaaacggcttgcctgactttatgagtcaaagtatgctgcacaattatcgctcttttatattagagcgttctgggatgcttcctagcatgtgttgtgcacttccttcagattttgtgcctatatattttagagagtgcccccctcccctgtggagccactgctacttactacgacttgctaactacctagcttaccactcagaccttatgacagattcaagcggcgaaggcctaatggagtgtcactgccgctgcaatctttgcaccccccaccgttctttggtttgcaatactgaactattaagtgaaagtcaagtcattggtaccttcgaaatgcagggaccgcagtctgacagcaatttcacgacgaacctaagacttacccctgggctttggacttctgcctacctgcgcaaatttgaaccccaagattaccacgcccacagtatcaatttttacgaagaccaatccaaacccccaaaagcgccactaacggcttgcgtcattacgcagggaaaaattctagcccaattgcatgctattaagcaagcgcgcgaagagtttttacttaaaaaaggacacggagtgtaccttgatccccaaaccggcgaggaactaaaccttccatcacctttgtgtgctactgcgtctccccattcgcagcatgtccccgaaagccgcaaaacaggctattgcgcagcaacgctcaaagaaacagcagcaacggcaggaaatctgggaggaagaatcttgggagagtcaggcagaggacgaggtcgaggacttggaagaatgggaggaggaggaggcggacagcctagacgaggatccagaggaggaggaggaaggttccaaggacggagcgaccgccgccaaaccgtcgctttcaaccaagccctctccaatgaaacccgctgtgagcaaatctcagaaagccaaccgtagatgggacaccattgaaaccagcgccgcaaacttgggtaagaatcgcaagcaggcgcgtcggggctactgctcatggcgggctcaccaaagtaatattgtagcctgctttcagcactgcggggggaatatctcatttgcaaggcggtatttgctataccatgatggagtggcgattccaaggaatgtcctccattactaccgtcatctctacagcccctttgaagagctcgacaaggaaccgacctgcaacagccaagcggcccactagaatcggcaacagcagcaacaaggaaagtcctgaggcgcgcgagttaagaaaacgcatttttcccactttatatgctatttttcagcagagtcgaggtcaagaacacgaactgaaaataaaaaaccgttccctgcgttcacttacccgcagctgtctctacctcaaaagcgaagatcagttgcaacgcaccttgcaggacgcagaagctctgttcaataaatactgctccctctcgcttaaagagtaaaaaaagcccgcgcgcggactttcaacaggcgggaaaagtgacgtcacaacaagatgagtaaagatattcccacgccttacatgtggagctttcaaccccaaatgggactggcggccggcgcggctcaagactattctagcaaaatgaattggttaagcgccggaccccacatgatttccagggtgaatggggtacgagcccggcgtaaccaaatactgctagaacaagccgctctcaccgctacaccacgtaatcaacttaaccctccctcttggccagctgccctgatatatcaggaaaatccccctcctaccactgtacttttgcctcgcgacgcccaggccgaagtccatatgactaacgctggggcacagcttgcgggcggtgcacgtcacagtttcaggtataaaggtcgcactgagccctatccgtctccagctataaaaagagtactcatcagagggaaaggtattcagctgaacgacgaagtcacatcgccattgggagtcagacccgacggagtgtttcagctcggagggtccggacgttcctcctttaccgctcgtcaagcctacctgacactacagagctcatcctcagctccgagatctggtggtattggaactctccaatttgtggaggaatttactccatctgtttacttcaatcctttttcgggctcgcctggacactatcctgacgccttcatacccaactttgacgcagtgagtgaatctgtggatggctatgattaatgtctaatggagcggctgacagagcgcggctgcgacatttagaccactgtcgccaacctcactgctttgctcgagacatctgtgtctttacctactttgagcttccagaggagcacccccaggggccagctcacggtgtcagaataacagttgaaaaaggaattgatacacacctcattaaatttttcaccaaacgcccgctattggtggaaaaagatcaaggaaatactatattaactttatattgcatttgtcctgttcccggattacatgaagatttctgctgtcatttgtgtgctgaatttaatcatctgtagtggcgctgtaccgcctgaagaagaacctaactgtcatccgcatttaagcaacattaaaatcaacctttcgatccctcatatcactcttcgctgcagttttttttccacacatctcacctggacctttaacggaaaacacgttaccaatacagatataaagtttaaactacacaaagaaaacatcactctatttcaacctattaacctgggatactaccgctgctcagctccaccctgtacgcaagcattttttgttgctccagttattgacaaacgccctgctccgacaacagctgctgtcactgagcacatcaccgaggcagtttctccttctaaaggtacagaggaaattgtgtacttttcaaactttacaaaccacttagttttaaattgttcctgttctaactccttaatttcatggtttgctaacagctctctgtgcaaaactttctaccaaggaaaacttttgtattctgctaaactcacattgtgtaaccagagcaccccttcccaccttactctattgccaccttttgttgccggtcgttacttttgcataggagctgcacgtactagcccctgtcaacagcattggaatttaacttactgtcccccaccagtgtcgccctttgtgatcaatactgaatatttagactataatcccttgcttgcttacggcggtctcgcagctcttattttattcctgatttctaacttgtttctagtgcaacatttgtattcatactaacaatgctttccatttttcttttatttctcttttctttaccttctggcttgtatgctcaaacagccgaaagaccactaaaagtcgtggtggaagctggccataatgtaacccttccccacctttctggttcacaccaaactggccatgttacttggctagtagagacatcagattatggttcagcttctccagacaacttcattttcagtggacaaaaactatgccagtttactgacagaaccatggtgtggccttattacaatttacattttaactgtgaaaattatgaccttaatctgttttggcttaaggtggaaaattcggctatttacaacgttaaaaatacagtcaatgcttctgaaacaaatatttactatgatttaagagtagtacaaatttttccacctaaatgcatcattacttcaaagtaccttacaaacgattattgtcacattacaattaactgcactaactctgattaccccaataaggttgtgtttaataatgtcagtcgatggtactacggatacggtaagggcagcccaacccttcccaactactttataactaactttaatgtttcaggtattactaaaagctttaatcacacttacccttttaatgagctctgtgattatcccacatcccaatctcaacacagtttaacacatacagtaagcacagtaatctttttaggaataattggcttcagcattttgattattatagcagcctttatttatctgtgctggcatagaaaatctttgtgtgtttctaaaacagaacctcttatgccgattccttactagttttcttttttcttacagtatggtgacggttcttctcatctttttatgcctgccagtcattttttcttcttcgacttttgccgcagtcagtgaccttgatcccgagtgtttagccccctttgcggtgtacctgattttcacatttgtgactgctacctgcgtctgcagtattattactctgctaatcacctcgctccaattttttgattactactacgtgagaattgtttaccgcagacaccacccccgttaccaaaaccctcaaattgcggctcttttgcagctccaaccatgaaaacagcattagttcttttctttatgttaatcccagtttgggctagttcttgtcaactacataaaccatggaattttttagattgttatactaaagaaacaaactacataggctgggtttatggaattatgtctggcttagtatttgtctcctctgtagtttctttacaactgtatgcgcgccttaattttagttggaataagtatactgatgatcttcccgaatatccaaacccccaggatgatttacccctaaatattgtatttccagagcccccgcgtcctccttctgttgttagctattttaagttcaccggtgaagatgattgaacctgatctagaaattgatggaagaatcaccgaacagaggctcctcactgatcgcgctaggcgacgccaacaggatcaaaaaaataaagagttaattgatttacaaaccgtgcatcagtgtaaaaaaggacttttttgcctggtaaaacaagctacccttcgctatgaatctttaccaggcaaagaacatcaactgtgctacacgctgcccactcagcgacaaacctttactgcaatggtgggctcggtacctattaaagtgtcccaacaagcaggagaacaagaaggctctattcggtgcctatgtgataaccctgaatgtttgtacactttaataaaaacactgtgcggtttaagaaatcttttaccaatgaattaaataaattacttaccggaaatctgaaaatacatcatggtctccgtgtactcttataaaatttccctcttcccaactgtcaaacctgacagacttgcaaacagcaaactttctccaaatcttaaatggaaggtcagattcttcttcccaatccctacccaccatcttcatcttttctagatgaagcgcagcagaacccagtatgctgaagaaacagaagaaaatgatgacttcaaccccgtttacccttttgacccatttgacacatcagacgtaccctttgttacacccccttttacttcttccaatggtcttcaagaaaaaccaccaggtgtattagcacttaattacaaagaccccattgtaactgaaaatggaacccttacactcaagctaggggacggaataaaacttaatgcccaaggtcaacttacagctagtaataatatcaatgttttggagccccttaccaacacctcacaaggtcttaaactttcttggagcgcccccctagcagtaaaggctagtgccctcacacttaacacaagagcgcccttaaccacaacggatgaaagcttagccttaataaccgcccctcccattacagtagagtcttcgcgtttgggcttggccaccatagcccctctaagcttagatggaggtggaaacctaggtttaaatctttctgctcccctggacgttagtaacaacaatttgcatctcaccactgaaactcccttagttgtaaattctagcggtgccctatctgttgctactgcagaccccataagtgttcgcaacaacgctcttaccctacctacggcagatccgttaatggtgagctccgatgggttgggaataagtgtcactagtcccattacagtaataaacggttccttagccttgtctacaactgctcccctcaacagcacaggatccactttaagtctgtctgttgccaatcctctgactatttcacaagacacattgactgtttccactggtaacggtcttcaagtgtcggggtctcaattagtaacaagaataggggatggtttaacattcgataatggggtcatgaaagtaaacgttgccgggggaatgagaacttctggcggtagaataattttagatgttaattatccctttgatgcgagcaataacctgtccttaagacggggattgggactaatttataaccaatctacaaactggaacttaacaactgatattagtaccgaaaaaggtttaatgtttagtggcaatcaaatagctcttaatgcaggtcaggggcttacatttaataatggccaacttagggttaagttgggagctggacttatttttgattcaaacaataacattgccttaggcagcagcagcaacactccatacgaccctctgacactgtggacaactcctgacccaccaccaaactgcagcctcatacaagagctagatgcaaaactcaccctgtgcttaacaaaaaacggatctattgttaatggcattgtaagtttagtgggtgttaagggtaatctcctaaatatccaaagtactactaccactgtaggagtgcatttagtgtttgatgaacagggaagattaatcacatcaacccctactgccctggttccccaagcttcgtggggatatagacaaggccaatcagtgtctaccaatactgttaccaatggtctaggttttatgcctaatgtgagtgcttaccctagaccaaatgccagtgaggctaaaagccaaatggtaagtctcacgtacttacagggagatacatctaaacctataacaatgaaagttgcatttaatggcattacgtcgctaaatggatactctttaacattcatgtggtcaggtctatcaaactatataaatcagcctttctctacaccatcctgctccttttcttacattacccaagaataaaaacacacacaaaacacaaattgcgtacttattgtttatttttttttttttttacactatacgcgtggttaaactgcctccttcccattttaccttgtatacctccctttccccctttgtagctgaaaacaactgcacttgaatatttcgacttaggttttttggcgttagcgtccacacagtttctttacgggcaaagcgagggtcggtgatggaaacgaatccctcgcccgcacagtcactcaagcggcattccccatccaaaaccaggtccatgattttatcctacaaaaagtaacaacagtcagtgtccatcagccgcccaaggattctctcgttgattataatctccaaataaaattgctcgatgatgcataattaaaccctttagcagttgctgacgataacgttcatgccgactatgttttagagggcgaacagtgttttcagcaattacttgaacaacttttaacattagcagtctggtacgacgagcgcaacagcgcatgcgtatctcacttaagtctttacaataatcacaacacagcactaacatgttatttaaaattccataattaaaggcgctccatccaaaactaactttttctaacgctaaccaggcatggccatcatacataattttaaagtaaattaaatggcgacctctaacaaaggtgcttcccacatacatcacctctttaggcattaaatggttaacaacctcccgataccaaaaacaccttttgttaattaaggcgccatatacggccattttgaaccagcgtcccaaaagcatcccagctgacatacactgtagtgaacccggacgctggcaatgacaatgaataagccaccgctcatgaccatgtaataattgagtaacttcaacatttatagtggcacaacacatacatacactcatgtattttttcaaaataaacatctcataatcagttagaatcatatcccacggtattggccattcctgcagcactgtaaaacctacacatgaaggaatgcctcttacctcacttacattatgtaaagtcagactattacactcaggccataaagaattttccgaagtactcaacgtagcttttgactgttcctcacagggcggtagttggtacttgttgtatggtgccaatctgtagcgataccgtctgtcgcgctgcatcgtaaacaacagacttgcgagcgtcttcgtacttaaaaaaacaaaaccacgtacgaccactggttatcgcacctcgtcctttttgtttgcagcgttggcgttccgtcaaaaaagcaaagtacaaccactctcgcaggcttgctaaaatgtattcagcttcaggtgttatcttcaaatcatgatgtttaataaagcgcagagtatccacacaggatgcatgggctaaaccaagccatgctatgcaggcagccgtgtcccgacttacaggaggaggaggaatacaaggtagaggcataaaaacttaatcaagacggtcagcaaggatttgaatgcgtaaatctcgcaggtggcagcgatcgcctccgctgtgctggtgaaagatcacagccagatcaaattgtaagcgattttccaaatgttcaacaacagcttctaaaagagccacagctctgattt

SEQ ID NO: 26 - DNA, 18 bp, Artificial Sequence
  Synthetic Oligonucleotide
cggtggaactgcagacag

SEQ ID NO: 27 - DNA, 20 bp, Artificial Sequence
  Synthetic Oligonucleotide
catcacggtttcgtaagagg

SEQ ID NO: 28 - DNA, 23 bp, Artificial Sequence
  Synthetic Oligonucleotide
cgctgaactccactgatactgct

SEQ ID NO: 29 - DNA, 22 bp, Artificial Sequence
  Synthetic Oligonucleotide
tggcttttcccgaggcaccacc

SEQ ID NO: 30 - DNA, 23 bp, Artificial Sequence
  Synthetic Oligonucleotide
cccgcccttacagatcacgggac

SEQ ID NO: 31 - DNA, 20 bp, Artificial Sequence
  Synthetic Oligonucleotide
tggactccccggatactgct

SEQ ID NO: 32 - DNA, 20 bp, Artificial Sequence
  Synthetic Oligonucleotide
tgccgttgcgcagcagtatc

SEQ ID NO: 33 - DNA, 22 bp, Artificial Sequence
  Synthetic Oligonucleotide
acagaccatcaatcttgacgac

SEQ ID NO: 34 - DNA, 21 bp, Artificial Sequence
  Synthetic Oligonucleotide
accatcacccgcgccttaaac

SEQ ID NO: 35 - DNA, 22 bp, Artificial Sequence
  Synthetic Oligonucleotide
gtaatggtcactcgctggactc

SEQ ID NO: 36 - DNA, 26 bp, Artificial Sequence
  Synthetic Oligonucleotide
cggcgacctgaaaaccatcctgcata

SEQ ID NO: 37 - DNA, 19 bp, Artificial Sequence
  Synthetic Oligonucleotide
acggtcactcgctggactc

SEQ ID NO: 38 - DNA, 21 bp, Artificial Sequence
  Synthetic Oligonucleotide
gatggtagtcaggatgagctg

SEQ ID NO: 39 - DNA, 23 bp, Artificial Sequence
  Synthetic Oligonucleotide
aatggtcatggtcaccgagaagt

SEQ ID NO: 40 - DNA, 19 bp, Artificial Sequence
  Synthetic Oligonucleotide
taacggtcacgcgctggac

SEQ ID NO: 41 - DNA, 26 bp, Artificial Sequence
  Synthetic Oligonucleotide
atacgagtgggtggagtttgagctgc

SEQ ID NO: 42 - DNA, 23 bp, Artificial Sequence
  Synthetic Oligonucleotide
tttagcctacaactatggagacc

SEQ ID NO: 43 - DNA, 23 bp, Artificial Sequence
  Synthetic Oligonucleotide
tttggcctacaactacggagatg

SEQ ID NO: 44 - DNA, 17 bp, Artificial Sequence
  Synthetic Oligonucleotide
tcttgcatcatgtcggg

SEQ ID NO: 45 - DNA, 19 bp, Artificial Sequence
  Synthetic Oligonucleotide
actccactgatactgctgc

SEQ ID NO: 46 - DNA, 22 bp, Artificial Sequence
  Synthetic Oligonucleotide
accagtacacctgctgcgaccc

SEQ ID NO: 47 - DNA, 23 bp, Artificial Sequence
  Synthetic Oligonucleotide
ccygcyctyacagatcacggrac

SEQ ID NO: 48 - DNA, 17 bp, Artificial Sequence
  Synthetic Oligonucleotide
  modified_base (2)..(3): m5c
  misc_feature (2)..(3): n is a, c, g, or t
  modified_base (15)..(15): m5c
  misc_feature (15)..(15): n is a, c, g, or t
anngttagtgaaaangt

SEQ ID NO: 49 - DNA, 17 bp, Artificial Sequence
  Synthetic Oligonucleotide
  modified_base (2)..(3): m5c
  misc_feature (2)..(3): n is a, c, g, or t
  modified_base (6)..(6): m5c
  misc_feature (6)..(6): n is a, c, g, or t
  modified_base (15)..(15): m5c
  misc_feature (15)..(15): n is a, c, g, or t
annatnagtgaaaangt

SEQ ID NO: 50 - DNA, 17 bp, Artificial Sequence
  Synthetic Oligonucleotide
  modified_base (2)..(2): m5c
  misc_feature (2)..(2): n is a, c, g, or t
gncttgtagacgtaggg

SEQ ID NO: 51 - DNA, 17 bp, Artificial Sequence
  Synthetic Oligonucleotide
  modified_base (2)..(3): m5c
  misc_feature (2)..(3): n is a, c, g, or t
  modified_base (11)..(11): m5c
  misc_feature (11)..(11): n is a, c, g, or t
gnnttgtaaangtaggg

SEQ ID NO: 52 - DNA, 17 bp, Artificial Sequence
  Synthetic Oligonucleotide
  modified_base (2)..(2): m5c
  misc_feature (2)..(2): n is a, c, g, or t
  modified_base (9)..(9): m5c
  misc_feature (9)..(9): n is a, c, g, or t
  modified_base (11)..(11): m5c
  misc_feature (11)..(11): n is a, c, g, or t
gntttgtanangtaagg

SEQ ID NO: 53 - DNA, 23 bp, Artificial Sequence
  Synthetic Oligonucleotide
  modified_base (2)..(2): m5c
  misc_feature (2)..(2): n is a, c, g, or t
  modified_base (5)..(5): m5c
  misc_feature (5)..(5): n is a, c, g, or t
  modified_base (7)..(7): m5c
  misc_feature (7)..(7): n is a, c, g, or t
  modified_base (9)..(9): m5c
  misc_feature (9)..(9): n is a, c, g, or t
cntgntntnacagatcacgggac

SEQ ID NO: 54 - DNA, 17 bp, Artificial Sequence
  Synthetic Oligonucleotide
  modified_base (2)..(3): m5c
  misc_feature (2)..(3): n is a, c, g, or t
  modified_base (6)..(6): m5c
  misc_feature (6)..(6): n is a, c, g, or t
  modified_base (15)..(15): m5c
  misc_feature (15)..(15): n is a, c, g, or t
anngtnagtgaaaangt

SEQ ID NO: 55 - DNA, 17 bp, Artificial Sequence
  Synthetic Oligonucleotide
  modified_base (2)..(3): m5c
  misc_feature (2)..(3): n is a, c, g, or t
  modified_base (5)..(6): propyne dU
  misc_feature (5)..(6): n is a, c, g, or t
  modified_base (9)..(9): propyne dU
  misc_feature (9)..(9): n is a, c, g, or t
  modified_base (15)..(15): propyne dC
  misc_feature (15)..(15): n is a, c, g, or t
anngnnagngaaaangt

SEQ ID NO: 56 - DNA, 17 bp, Artificial Sequence
  Synthetic Oligonucleotide
  modified_base (2)..(3): m5c
  misc_feature (2)..(3): n is a, c, g, or t
  modified_base (5)..(5): propyne dU
  misc_feature (5)..(6): n is a, c, g, or t
  modified_base (6)..(6): m5c
  modified_base (9)..(9): propyne dU
  misc_feature (9)..(9): n is a, c, g, or t
  modified_base (15)..(15): propyne dC
  misc_feature (15)..(15): n is a, c, g, or t
annannagngaaaangt

SEQ ID NO: 57 - DNA, 17 bp, Artificial Sequence
  Synthetic Oligonucleotide
  modified_base (2)..(3): propyne dC
  misc_feature (2)..(5): n is a, c, g, or t
  modified_base (4)..(5): propyne dU
  modified_base (7)..(7): propyne dU
  misc_feature (7)..(7): n is a, c, g, or t
  modified_base (11)..(11): propyne dC
  misc_feature (11)..(11): n is a, c, g, or t
  modified_base (13)..(13): propyne dU
  misc_feature (13)..(13): n is a, c, g, or t
gnnnngnagangnaggg

SEQ ID NO: 58 - DNA, 17 bp, Artificial Sequence
  Synthetic Oligonucleotide
  modified_base (2)..(3): propyne dC
  misc_feature (2)..(5): n is a, c, g, or t
  modified_base (4)..(5): propyne dU
  modified_base (7)..(7): propyne dU
  misc_feature (7)..(7): n is a, c, g, or t
  modified_base (11)..(11): propyne dC
  misc_feature (11)..(11): n is a, c, g, or t
  modified_base (13)..(13): propyne dU
  misc_feature (13)..(13): n is a, c, g, or t
gnnnngnaaangnaggg

SEQ ID NO: 59 - DNA, 17 bp, Artificial Sequence
  Synthetic Oligonucleotide
  modified_base (2)..(2): propyne dC
  misc_feature (2)..(5): n is a, c, g, or t
  modified_base (3)..(5): propyne dU
  modified_base (7)..(7): propyne dU
  misc_feature (7)..(7): n is a, c, g, or t
  modified_base (9)..(9): propyne dC
  misc_feature (9)..(9): n is a, c, g, or t
  modified_base (11)..(11): propyne dC
  misc_feature (11)..(11): n is a, c, g, or t
  modified_base (13)..(13): propyne dU
  misc_feature (13)..(13): n is a, c, g, or t
gnnnngnanangnaagg

SEQ ID NO: 60 - DNA, 17 bp, Artificial Sequence
  Synthetic Oligonucleotide
  modified_base (2)..(2): propyne dC
  misc_feature (2)..(5): n is a, c, g, or t
  modified_base (3)..(5): propyne dU
  modified_base (7)..(7): propyne dU
  misc_feature (7)..(7): n is a, c, g, or t
  modified_base (11)..(11): propyne dC
  misc_feature (11)..(11): n is a, c, g, or t
  modified_base (13)..(13): propyne dU
  misc_feature (13)..(13): n is a, c, g, or t
gnnnngnggangnaggg

SEQ ID NO: 61 - DNA, 23 bp, Artificial Sequence
  Synthetic Oligonucleotide
  modified_base (2)..(2): m5c
  misc_feature (2)..(2): n is a, c, g, or t
  modified_base (5)..(5): m5c
  misc_feature (5)..(5): n is a, c, g, or t
  modified_base (7)..(7): m5c
  misc_feature (7)..(7): n is a, c, g, or t
  modified_base (9)..(9): m5c
  misc_feature (9)..(9): n is a, c, g, or t
  modified_base (11)..(11): m5c
  misc_feature (11)..(11): n is a, c, g, or t
  modified_base (16)..(16): m5c
  misc_feature (16)..(16): n is a, c, g, or t
  modified_base (18)..(18): m5c
  misc_feature (18)..(18): n is a, c, g, or t
  modified_base (23)..(23): m5c
  misc_feature (23)..(23): n is a, c, g, or t
cntgntntnanagatnangggan

SEQ ID NO: 62 - DNA, 23 bp, Artificial Sequence
  Synthetic Oligonucleotide
  modified_base (9)..(9): m5c
  misc_feature (9)..(9): n is a, c, g, or t
  modified_base (11)..(11): m5c
  misc_feature (11)..(11): n is a, c, g, or t
  modified_base (16)..(16): m5c
  misc_feature (16)..(16): n is a, c, g, or t
  modified_base (18)..(18): m5c
  misc_feature (18)..(18): n is a, c, g, or t
cccgccctnanagatnanggaac

SEQ ID NO: 63 - DNA, 23 bp, Artificial Sequence
  Synthetic Oligonucleotide
  modified_base (7)..(7): m5c
  misc_feature (7)..(7): n is a, c, g, or t
  modified_base (9)..(9): m5c
  misc_feature (9)..(9): n is a, c, g, or t
  modified_base (11)..(11): m5c
  misc_feature (11)..(11): n is a, c, g, or t
  modified_base (16)..(16): m5c
  misc_feature (16)..(16): n is a, c, g, or t
  modified_base (18)..(18): m5c
  misc_feature (18)..(18): n is a, c, g, or t
cccgctntnanagatnangggac

SEQ ID NO: 64 - DNA, 20 bp, Artificial Sequence
  Synthetic Oligonucleotide
  modified_base (1)..(1): m5c
  misc_feature (1)..(1): n is a, c, g, or t
  modified_base (3)..(3): propyne dU
  misc_feature (3)..(4): n is a, c, g, or t
  modified_base (4)..(4): m5c
  modified_base (6)..(6): m5c
  misc_feature (6)..(6): n is a, c, g, or t
  modified_base (9)..(11): propyne dU
  misc_feature (9)..(12): n is a, c, g, or t
  modified_base (12)..(12): m5c
  modified_base (14)..(14): propyne dU
  misc_feature (14)..(14): n is a, c, g, or t
nannanggnnnngnaagagg

SEQ ID NO: 65 - DNA, 22 bp, Artificial Sequence
  Synthetic Oligonucleotide
  modified_base (4)..(4): m5c
  misc_feature (4)..(4): n is a, c, g, or t
  modified_base (9)..(11): m5c
  misc_feature (9)..(11): n is a, c, g, or t
tggnttttnnngaggcaccacc

SEQ ID NO: 66 - DNA, 23 bp, Artificial Sequence
  Synthetic Oligonucleotide
  modified_base (6)..(7): m5c
  misc_feature (6)..(7): n is a, c, g, or t
  modified_base (11)..(11): m5c
  misc_feature (11)..(11): n is a, c, g, or t
  modified_base (16)..(16): m5c
  misc_feature (16)..(16): n is a, c, g, or t
  modified_base (18)..(18): m5c
  misc_feature (18)..(18): n is a, c, g, or t
  modified_base (23)..(23): m5c
  misc_feature (23)..(23): n is a, c, g, or t
cccgcnnttanagatnangggan

SEQ ID NO: 67 - DNA, 22 bp, Artificial Sequence
  Synthetic Oligonucleotide
  modified_base (9)..(9): m5c
  misc_feature (9)..(9): n is a, c, g, or t
  modified_base (11)..(11): m5c
  misc_feature (11)..(11): n is a, c, g, or t
  modified_base (13)..(13): m5c
  misc_feature (13)..(13): n is a, c, g, or t
  modified_base (15)..(15): m5c
  misc_feature (15)..(15): n is a, c, g, or t
gtaatggtnantngntggactc

SEQ ID NO: 68 - DNA, 26 bp, Artificial Sequence
  Synthetic Oligonucleotide
  modified_base (7)..(8): m5c
  misc_feature (7)..(8): n is a, c, g, or t
  modified_base (15)..(16): m5c
  misc_feature (15)..(16): n is a, c, g, or t
  modified_base (19)..(20): m5c
  misc_feature (19)..(20): n is a, c, g, or t
cggcganntgaaaannatnntgcata

SEQ ID NO: 69 - DNA, 26 bp, Artificial Sequence
  Synthetic Oligonucleotide
  modified_base (4)..(4): m5c
  misc_feature (4)..(4): n is a, c, g, or t
  modified_base (8)..(8): propyne dU
  misc_feature (8)..(8): n is a, c, g, or t
  modified_base (12)..(12): propyne dU
  misc_feature (12)..(12): n is a, c, g, or t
  modified_base (23)..(23): m5c
  misc_feature (23)..(23): n is a, c, g, or t
atangagngggnggagtttgagntgc

SEQ ID NO: 70 - DNA, 23 bp, Artificial Sequence
  Synthetic Oligonucleotide
  modified_base (6)..(7): m5c
  misc_feature (6)..(8): n is a, c, g, or t
  modified_base (8)..(8): propyne dU
  modified_base (10)..(10): m5c
  misc_feature (10)..(10): n is a, c, g, or t
  modified_base (13)..(13): m5c
  misc_feature (13)..(14): n is a, c, g, or t
  modified_base (14)..(14): propyne dU
  modified_base (22)..(22): m5c
  misc_feature (22)..(22): n is a, c, g, or t
tttagnnnanaannatggaganc

SEQ ID NO: 71 - DNA, 23 bp, Artificial Sequence
  Synthetic Oligonucleotide
  modified_base (6)..(7): m5c
  misc_feature (6)..(7): n is a, c, g, or t
  modified_base (10)..(10): m5c
  misc_feature (10)..(10): n is a, c, g, or t
tttggnntanaactacggagatg

SEQ ID NO: 72 - DNA, 17 bp, Artificial Sequence
  Synthetic Oligonucleotide
  modified_base (2)..(2): m5c
  misc_feature (2)..(2): n is a, c, g, or t
  modified_base (6)..(6): m5c
  misc_feature (6)..(6): n is a, c, g, or t
  modified_base (9)..(9): m5c
  misc_feature (9)..(9): n is a, c, g, or t
  modified_base (14)..(14): m5c
  misc_feature (14)..(14): n is a, c, g, or t
tnttgnatnatgtnggg

SEQ ID NO: 73 - DNA, 19 bp, Artificial Sequence
  Synthetic Oligonucleotide
  modified_base (2)..(2): m5c
  misc_feature (2)..(2): n is a, c, g, or t
  modified_base (4)..(5): m5c
  misc_feature (4)..(5): n is a, c, g, or t
  modified_base (7)..(7): m5c
  misc_feature (7)..(8): n is a, c, g, or t
  modified_base (8)..(8): propyne dU
  modified_base (11)..(11): propyne dU
  misc_feature (11)..(11): n is a, c, g, or t
  modified_base (13)..(13): m5c
  misc_feature (13)..(13): n is a, c, g, or t
  modified_base (16)..(16): m5c
  misc_feature (16)..(16): n is a, c, g, or t
antnnannganantgntgc

SEQ ID NO: 74 - DNA, 22 bp, Artificial Sequence
  Synthetic Oligonucleotide
  modified_base (8)..(8): m5c
  misc_feature (8)..(8): n is a, c, g, or t
  modified_base (10)..(11): m5c
  misc_feature (10)..(11): n is a, c, g, or t
  modified_base (14)..(14): m5c
  misc_feature (14)..(14): n is a, c, g, or t
accagtananntgntgcgaccc

(19) United States
(12) Patent Application Publication
(10) Pub. No.: US 2024/0218469 A1
(43) Pub. Date: Jul. 4, 2024

(54) COMPOSITIONS AND METHODS FOR DETECTING HUMAN ADENOVIRUS NUCLEIC ACID

(71) Applicant: Gen-Probe Incorporated, San Diego, CA (US)

(72) Inventor: Joshua Kalani MOBERLY, San Diego, CA (US)

(21) Appl. No.: 18/558,346

(22) PCT Filed: May 13, 2022
(86) PCT No.: PCT/US2022/029310

§ 371 Date: Oct. 31, 2023

Related U.S. Application Data
(60) Provisional application No. 63/188,785, filed on May 14, 2021.

(51) Int. Cl. C12Q 1/70; C12Q 1/6844

(52) U.S. Cl. CPC C12Q 1/701; C12Q 1/6844

(57) ABSTRACT

Nucleic acid oligomers, including amplification oligomers and detection probes, for detection of human adenovirus nucleic acid by binding to the penton gene. Also disclosed are methods of specific nucleic acid amplification and detection using the disclosed oligomers, as well as corresponding formulations, reaction mixtures, and kits and related methods for preparing aqueous reaction mixtures from dried formulations. Nucleic acid oligomers and methods for nucleic acid amplification and detection of one or more adenovirus subgroups A, B, C, D, E and F are also disclosed.

### REFERENCE TO SEQUENCE LISTING

The instant application contains a Sequence Listing which has been submitted in ASCII format via EFS-Web and is hereby incorporated by reference in its entirety. Said ASCII Copy, created on May 13, 2022, is named “DIA-0142-02_Seq_List_ST25” and is [72, 124] bytes in size.

### BACKGROUND

Adenovirus may cause infections in a number of different organs including the gastrointestinal tract, the upper respiratory tract, and the eyes. In individuals with a properly functioning immune system, Adenovirus infections are not typically associated with life-threatening disease. However, adenovirus can cause serious infection in immuno-compromised patients—such as HIV-positive individuals and in patients receiving bone marrow transplants. More than 50 different human adenovirus serotypes have been identified. On the basis of various properties of adenovirus, they have been divided into seven major subgroups (subgenera or subtypes A-G). Six of these subgroups (A-F) are considered clinically relevant.

Early approaches for detecting adenovirus detection relied mainly on serological tests and cell culture. In immunosuppressed patients, however, the use of serological tests is limited due to the impaired immune response, and evaluation of positive cultures is a relatively slow method. The introduction of PCR-based assays has provided new methods for the rapid, specific, and sensitive detection of adenovirus. Many of these diagnostic approaches, however, do not effectively cover all adenovirus serotypes or use low stringency conditions to permit detection of the genetically highly diverse adenoviruses.

The homology of adenovirus DNA sequences between different species is low. Even conserved regions within the adenovirus genome display only limited homology between adenoviruses from different species. In many instances, considerable differences in DNA sequence even exist between serotypes belonging to the same species. These facts underscore the difficulty to develop molecular tests that facilitate reliable screening for adenovirus infections with the required broad specificity.

While several adenovirus assays are currently on the market, due to the highly variable genomic sequence, usually only subgroups B, C, and E (responsible for most of the respiratory infections) are detected. Tissue transplant patients are highly susceptible to severe infections and transplant failures due to lingering pathogens, and a lack of effective assays to test all clinically relevant adenovirus subtypes leaves transplant patients at risk for infection and/or transplant failure.

A molecular based assay is required to permit the sensitive and specific detection of adenovirus subgroups A-F.

### SUMMARY

In some aspects, the present invention provides a composition or kit for determining the presence or absence of human adenovirus in a sample. In one such aspect, the composition or kit generally includes at least two amplification oligomers capable of amplifying a target region of a human adenovirus target nucleic acid. In another, non-mutually exclusive aspect, the composition or kit generally includes at least one detection probe oligomer capable of hybridizing to a target region of a human adenovirus target nucleic acid or to an amplicon of said target region.

In another aspect, the present invention provides a formulation for amplifying a target region of a human adenovirus target nucleic acid in a sample. The formulation generally includes (i) at least two amplification oligomers capable of amplifying a target region of a human adenovirus target nucleic acid, and (ii) a buffer. In some embodiments, the formulation is a dried composition. In a related aspect, the present invention provides a kit comprising such a dried composition for amplifying human adenovirus nucleic acid. In another related aspect, the present invention provides a method for preparing an aqueous reaction mixture for determining the presence or absence of human adenovirus in a sample, wherein the method generally includes the step of combining the dried composition with an aqueous reconstitution reagent.

In another aspect, the present invention provides a formulation for detecting human adenovirus in a sample. The formulation generally includes (i) at least one detection probe oligomer capable of hybridizing to a target region of a human adenovirus target nucleic acid or to an amplicon of said target region, and (ii) a buffer. In some embodiments, the formulation is a dried composition. In a related aspect, the present invention provides a kit comprising such a dried composition for detecting human adenovirus. In another related aspect, the present invention provides a method for preparing an aqueous reaction mixture for detecting human adenovirus in a sample, wherein the method generally includes the step of combining the dried composition with an aqueous reconstitution reagent.

In yet another aspect, the present invention provides a method for determining the presence or absence of human adenovirus in a sample. The method generally includes performing an in vitro nucleic acid amplification reaction, utilizing at least two amplification oligomers capable of amplifying a target region of a human adenovirus target nucleic acid, to generate an amplification product corresponding to the human adenovirus target region, and detecting the presence or absence of the amplification product.

Representative embodiments of these aspects are further set forth below.

### Embodiments

Embodiment 1. A combination or kit for determining the presence or absence of human adenovirus in a sample, the composition or kit comprising:
at least two amplification oligomers capable of amplifying a target region of a human adenovirus target nucleic acid, wherein
(a) at least one amplification oligomer comprises a target-hybridizing sequence as shown in SEQ ID NO:18, or an RNA equivalent or a DNA/RNA chimeric thereof and including from 0 to 9 nucleotide analogs; and
(b) at least one amplification oligomer is selected from the group consisting of
   (i) an oligomer comprising a target-hybridizing sequence as shown in SEQ ID NO:21, or an RNA equivalent or a DNA/RNA chimeric thereof and including from 0 to 9 nucleotide analogs, and
   (ii) an oligomer comprising a target-hybridizing sequence as shown in SEQ ID NO:20, or an RNA equivalent or a DNA/RNA chimeric thereof and including from 0 to 9 nucleotide analogs.

Embodiment 2. The composition or kit of embodiment 1, wherein the target-hybridizing sequence of (a) is SEQ ID NO:3, SEQ ID NO:4, or SEQ ID NO:5, or an RNA equivalent or a DNA/RNA equivalent thereof and including from 0 to 9 nucleotide analogs.

Embodiment 3. The composition or kit of any one of embodiment 1 or 2, wherein the composition or kit comprises at least two amplification oligomers as in (a).

Embodiment 4. The composition or kit of embodiment 3, wherein the composition or kit comprises first, second, and third amplification oligomers as in (a), wherein
the first amplification oligomer as in (a) comprises a target-hybridizing sequence as shown in SEQ ID NO:3, or an RNA equivalent or a DNA/RNA chimeric thereof and including from 0 to 9 nucleotide analogs,
the second amplification oligomer as in (a) comprises a target-hybridizing sequence as shown in SEQ ID NO:4, or an RNA equivalent or a DNA/RNA chimeric thereof and including from 0 to 9 nucleotide analogs, and
the third amplification oligomer as in (a) comprises a target-hybridizing sequence as shown in SEQ ID NO:5, or an RNA equivalent or a DNA/RNA chimeric thereof and including from 0 to 9 nucleotide analogs.

Embodiment 5. The composition or kit of any one of embodiments 1 to 4, wherein the composition or kit comprises the at least one amplification oligomer of (b)(i).

Embodiment 6. The composition or kit of embodiment 5, wherein the target-hybridizing sequence of (b)(i) is SEQ ID NO:6, SEQ ID NO:7, SEQ ID NO:8, or SEQ ID NO:9, or an RNA equivalent or a DNA/RNA equivalent thereof and including from 0 to 9 nucleotide analogs.

Embodiment 7. The composition or kit of any one of embodiments 1 to 6, wherein the composition or kit comprises at least two amplification oligomers as in (b)(i).

Embodiment 8. The composition or kit of embodiment 7, wherein the composition or kit comprises first, second, third, and fourth amplification oligomers as in (b)(i), wherein
the first amplification oligomer as in (b)(i) comprises a target-hybridizing sequence as shown in SEQ ID NO:6, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:7, or an RNA equivalent or a DNA/RNA chimeric thereof and including from 0 to 9 nucleotide analogs,
the third amplification oligomer as in (b)(i) comprises a target-hybridizing sequence as shown in SEQ ID NO:8, or an RNA equivalent or a DNA/RNA chimeric thereof and including from 0 to 9 nucleotide analogs, and
the fourth amplification oligomer as in (b)(i) comprises a target-hybridizing sequence as shown in SEQ ID NO:9, or an RNA equivalent or a DNA/RNA chimeric thereof and including from 0 to 9 nucleotide analogs.

Embodiment 9. The composition or kit of any one of embodiments 1 to 4, wherein the composition or kit comprises the at least one amplification oligomer of (b)(ii).

Embodiment 10. The composition or kit of embodiment 9, wherein the target-hybridizing sequence of (b)(ii) is SEQ ID NO:10, SEQ ID NO:11, SEQ ID NO: 12, or SEQ ID NO: 13, or an RNA equivalent or a DNA/RNA equivalent thereof and including from 0 to 9 nucleotide analogs.

Embodiment 11. The composition or kit of any one of embodiments 1 to 10, wherein the composition or kit comprises at least two amplification oligomers as in (b)(ii).

Embodiment 12. The composition or kit of embodiment 11, wherein the composition or kit comprises first, second, third, and fourth amplification oligomers as in (b)(ii), wherein
the first amplification oligomer as in (b)(ii) comprises a target-hybridizing sequence as shown in SEQ ID NO: 10, or an RNA equivalent or a DNA/RNA chimeric thereof and including from 0 to 9 nucleotide analogs,
the second amplification oligomer as in (b)(ii) comprises a target-hybridizing sequence as shown in SEQ ID NO:11, or an RNA equivalent or a DNA/RNA chimeric thereof and including from 0 to 9 nucleotide analogs,
the third amplification oligomer as in (b)(ii) comprises a target-hybridizing sequence as shown in SEQ ID NO:12, or an RNA equivalent or a DNA/RNA chimeric thereof and including from 0 to 9 nucleotide analogs, and
the fourth amplification oligomer as in (b)(ii) comprises a target-hybridizing sequence as shown in SEQ ID NO: 13, or an RNA equivalent or a DNA/RNA chimeric thereof and including from 0 to 9 nucleotide analogs.

Embodiment 13. The composition or kit of any one of embodiments 1 to 12, further comprising at least one detection probe oligomer comprising a target-hybridizing sequence configured to specifically hybridize to an amplification product amplifiable by the at least two amplification oligomers.

Embodiment 14. The composition or kit of embodiment 13, wherein the at least one detection probe oligomer target-hybridizing sequence is a sequence as shown in SEQ ID NO:19, or an RNA equivalent or a DNA/RNA chimeric thereof and including from 0 to 9 nucleotide analogs.

Embodiment 15. The composition or kit of embodiment 14, wherein the at least one detection probe oligomer target-hybridizing sequence is SEQ ID NO:15, SEQ ID NO: 16, or SEQ ID NO:17, or an RNA equivalent or a DNA/RNA equivalent thereof and including from 0 to 9 nucleotide analogs.

Embodiment 16. The composition or kit of any one of embodiments 13 to 15 wherein the at least one detection probe oligomer comprises a detectable label.

Embodiment 17. The composition or kit of embodiment 16, wherein the detectable label is a chemiluminescent or fluorescent label.

Embodiment 18. The composition or kit of embodiment 17, wherein the detectable label is a fluorescent label and the at least one detection probe oligomer further comprises a non-fluorescent quencher.

Embodiment 19. The composition or kit of any one of embodiments 13 to 15, wherein the composition or kit comprises at least two detection probe oligomers.

Embodiment 20. The composition or kit of embodiment 19, wherein the composition or kit comprises first, second, and third detection probe oligomers, wherein
the first detection probe oligomer comprises a target-hybridizing sequence as shown in SEQ ID NO: 15, or an RNA equivalent or a DNA/RNA chimeric thereof and including from 0 to 9 nucleotide analogs,
the second detection probe oligomer comprises a target-hybridizing sequence as shown in SEQ ID NO:16, or an RNA equivalent or a DNA/RNA chimeric thereof and including from 0 to 9 nucleotide analogs, and
the third detection probe oligomer comprises a target-hybridizing sequence as shown in SEQ ID NO:17, or an RNA equivalent or a DNA/RNA chimeric thereof and including from 0 to 9 nucleotide analogs.

Embodiment 21. The composition or kit of embodiment 19, wherein the composition or kit comprises first and second detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs, and
the second detection probe oligomer comprises a target-hybridizing sequence as shown in SEQ ID NO:16, or an RNA equivalent or a DNA/RNA chimeric thereof and including from 0 to 9 nucleotide analogs.

Embodiment 22. The composition or kit of embodiment 21, wherein the composition or kit further comprises a third detection probe oligomer.

Embodiment 23. The composition or kit of any one of embodiments 19 to 22, wherein each detection probe oligomer comprises a detectable label.

Embodiment 24. The composition or kit of embodiment 23, wherein the detectable label is a chemiluminescent or fluorescent label.

Embodiment 25. The composition or kit of embodiment 24, wherein the detectable label is a fluorescent label and each detection probe oligomer further comprises a non-fluorescent quencher.

Embodiment 26. The composition or kit of any one of embodiments 1 to 25, wherein one or more cytosines of at least one of the at least two amplification oligomers are 5-methyl cytosine.

Embodiment 27. The composition or kit of any one of embodiments 13 to 18, wherein one or more cytosines of the at least one detection probe oligomer are 5-methyl cytosine.

Embodiment 28. The composition or kit of any one of embodiments 19 to 25, wherein one or more cytosines of at least one of the at least two detection probe oligomers are 5-methyl cytosine.

Embodiment 29. A composition or kit for detecting human adenovirus in a sample, the composition or kit comprising: at least one detection probe oligomer comprising a target-hybridizing sequence as shown in SEQ ID NO: 19, or an RNA equivalent or a DNA/RNA chimeric thereof and including from 0 to 9 nucleotide analogs.

Embodiment 30. The composition or kit of embodiment 29, wherein the at least one detection probe oligomer target-hybridizing sequence is SEQ ID NO: 15, SEQ ID NO: 16, or SEQ ID NO:17, or an RNA equivalent or a DNA/RNA equivalent thereof and including from 0 to 9 nucleotide analogs.

Embodiment 31. The composition or kit of embodiment 29 or 30, wherein the at least one detection probe oligomer comprises a detectable label.

Embodiment 32. The composition or kit of embodiment 31, wherein the detectable label is a chemiluminescent or fluorescent label.

Embodiment 33. The composition or kit of embodiment 32, wherein the detectable label is a fluorescent label and the at least one detection probe oligomer further comprises a non-fluorescent quencher.

Embodiment 34. The composition or kit of embodiment 29 or 30, wherein the composition or kit comprises at least two detection probe oligomers.

Embodiment 35. The composition or kit of embodiment 34, wherein the composition or kit comprises first, second, and third detection probe oligomers, wherein
the first detection probe oligomer comprises a target-hybridizing sequence as shown in SEQ ID NO: 15, or an RNA equivalent or a DNA/RNA chimeric thereof and including from 0 to 9 nucleotide analogs,
the second detection probe oligomer comprises a target-hybridizing sequence as shown in SEQ ID NO:16, or an RNA equivalent or a DNA/RNA chimeric thereof and including from 0 to 9 nucleotide analogs, and
the third detection probe oligomer comprises a target-hybridizing sequence as shown in SEQ ID NO:17, or an RNA equivalent or a DNA/RNA chimeric thereof and including from 0 to 9 nucleotide analogs.

Embodiment 36. The composition or kit of embodiment 34, wherein the composition or kit comprises first and second detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs, and
the second detection probe oligomer comprises a target-hybridizing sequence as shown in SEQ ID NO:16, or an RNA equivalent or a DNA/RNA chimeric thereof and including from 0 to 9 nucleotide analogs.

Embodiment 37. The composition or kit of embodiment 36, wherein the composition or kit further comprises a third detection probe oligomer.

Embodiment 38. The composition or kit of any one of embodiments 33 to 36, wherein each detection probe oligomer comprises a detectable label.

Embodiment 39. The composition or kit of embodiment 38, wherein the detectable label is a chemiluminescent or fluorescent label.

Embodiment 40. The composition or kit of embodiment 39, wherein the detectable label is a fluorescent label and each detection probe oligomer further comprises a non-fluorescent quencher.

Embodiment 41. The composition or kit of any one of embodiments 29 to 33, wherein one or more cytosines of the at least one detection probe oligomer are 5-methyl cytosine.

Embodiment 42. The composition or kit of any one of embodiments 34 to 40, wherein one or more cytosines of at least one of the at least two detection probe oligomers are 5-methyl cytosine.

Embodiment 43. A formulation for amplifying a target region of a human adenovirus target nucleic acid in a sample, the formulation comprising:
(a) at least one amplification oligomer comprising a target-hybridizing sequence as shown in SEQ ID NO:18, or an RNA equivalent or a DNA/RNA chimeric thereof and including from 0 to 9 nucleotide analogs;
(b) at least one amplification oligomer selected from the group consisting of (i) an oligomer comprising a target-hybridizing sequence as shown in SEQ ID NO:21, or an RNA equivalent or a DNA/RNA chimeric thereof and including from 0 to 9 nucleotide analogs, and (ii) an oligomer comprising a target-hybridizing sequence as shown in SEQ ID NO:20, or an RNA equivalent or a DNA/RNA chimeric thereof and including from 0 to 9 nucleotide analogs; and
(c) a buffer.

Embodiment 44. The formulation of embodiment 43, wherein the target-hybridizing sequence of (a) is SEQ ID NO:3, SEQ ID NO:4, or SEQ ID NO:5, or an RNA equivalent or a DNA/RNA equivalent thereof and including from 0 to 9 nucleotide analogs.

Embodiment 45. The formulation of any one of embodiment 43 or 44, wherein the formulation comprises at least two amplification oligomers as in (a).

Embodiment 46. The formulation of embodiment 45, wherein the formulation comprises first, second, and third amplification oligomers as in (a), wherein
the first amplification oligomer as in (a) comprises a target-hybridizing sequence as shown in SEQ ID NO:3, or an RNA equivalent or a DNA/RNA chimeric thereof and including from 0 to 9 nucleotide analogs,
the second amplification oligomer as in (a) comprises a target-hybridizing sequence as shown in SEQ ID NO:4, or an RNA equivalent or a DNA/RNA chimeric thereof and including from 0 to 9 nucleotide analogs, and
the third amplification oligomer as in (a) comprises a target-hybridizing sequence as shown in SEQ ID NO:5, or an RNA equivalent or a DNA/RNA chimeric thereof and including from 0 to 9 nucleotide analogs.

Embodiment 47. The formulation of any one of embodiments 43 to 46, wherein the formulation comprises the at least one amplification oligomer of (b)(i).

Embodiment 48. The formulation of embodiment 47, wherein the target-hybridizing sequence of (b)(i) is SEQ ID NO:6, SEQ ID NO:7, SEQ ID NO:8, or SEQ ID NO:9, or an RNA equivalent or a DNA/RNA equivalent thereof and including from 0 to 9 nucleotide analogs.

Embodiment 49. The formulation of any one of embodiments 43 to 48, wherein the formulation comprises at least two amplification oligomers as in (b)(i).

Embodiment 50. The formulation of embodiment 49, wherein the formulation comprises first, second, third, and fourth amplification oligomers as in (b)(i), wherein
the first amplification oligomer as in (b)(i) comprises a target-hybridizing sequence as shown in SEQ ID NO:6, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:7, or an RNA equivalent or a DNA/RNA chimeric thereof and including from 0 to 9 nucleotide analogs,
the third amplification oligomer as in (b)(i) comprises a target-hybridizing sequence as shown in SEQ ID NO:8, or an RNA equivalent or a DNA/RNA chimeric thereof and including from 0 to 9 nucleotide analogs, and
the fourth amplification oligomer as in (b)(i) comprises a target-hybridizing sequence as shown in SEQ ID NO:9, or an RNA equivalent or a DNA/RNA chimeric thereof and including from 0 to 9 nucleotide analogs.

Embodiment 51. The formulation of any one of embodiments 43 to 46, wherein the formulation comprises the at least one amplification oligomer of (b)(ii).

Embodiment 52. The formulation of embodiment 51, wherein the target-hybridizing sequence of (b)(ii) is SEQ ID NO:10, SEQ ID NO:11, SEQ ID NO:12, or SEQ ID NO: 13, or an RNA equivalent or a DNA/RNA equivalent thereof and including from 0 to 9 nucleotide analogs.

Embodiment 53. The formulation of any one of embodiments 43 to 52, wherein the composition or kit comprises at least two amplification oligomers as in (b)(ii).

Embodiment 54. The formulation of embodiment 53, wherein the formulation comprises first, second, third, and fourth amplification oligomers as in (b)(ii), wherein
the first amplification oligomer as in (b)(ii) comprises a target-hybridizing sequence as shown in SEQ ID NO: 10, or an RNA equivalent or a DNA/RNA chimeric thereof and including from 0 to 9 nucleotide analogs,
the second amplification oligomer as in (b)(ii) comprises a target-hybridizing sequence as shown in SEQ ID NO:11, or an RNA equivalent or a DNA/RNA chimeric thereof and including from 0 to 9 nucleotide analogs,
the third amplification oligomer as in (b)(ii) comprises a target-hybridizing sequence as shown in SEQ ID NO: 12, or an RNA equivalent or a DNA/RNA chimeric thereof and including from 0 to 9 nucleotide analogs, and
the fourth amplification oligomer as in (b)(ii) comprises a target-hybridizing sequence as shown in SEQ ID NO: 13, or an RNA equivalent or a DNA/RNA chimeric thereof and including from 0 to 9 nucleotide analogs.

Embodiment 55. The formulation of any one of embodiments 43 to 54, further comprising at least one detection probe oligomer comprising a target-hybridizing sequence configured to specifically hybridize to an amplification product amplifiable by the at least two amplification oligomers.

Embodiment 56. The formulation of embodiment 55, wherein the at least one detection probe oligomer target-hybridizing sequence is a sequence as shown in SEQ ID NO:19, or an RNA equivalent or a DNA/RNA chimeric thereof and including from 0 to 9 nucleotide analogs.

Embodiment 57. The formulation of embodiment 56, wherein the at least one detection probe oligomer target-hybridizing sequence is SEQ ID NO:15, SEQ ID NO:16, or SEQ ID NO:17, or an RNA equivalent or a DNA/RNA equivalent thereof and including from 0 to 9 nucleotide analogs.

Embodiment 58. The formulation of any one of embodiments 55 to 57, wherein the at least one detection probe oligomer comprises a detectable label.

Embodiment 59. The formulation of embodiment 58, wherein the detectable label is a chemiluminescent or fluorescent label.

Embodiment 60. The formulation of embodiment 59, wherein the detectable label is a fluorescent label and the at least one detection probe oligomer further comprises a non-fluorescent quencher.

Embodiment 61. The formulation of any one of embodiments 55 to 57, wherein the formulation comprises at least two detection probe oligomers.

Embodiment 62. The formulation of embodiment 61, wherein the formulation comprises first, second, and third detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs,
the second detection probe oligomer comprises a target-hybridizing sequence as shown in SEQ ID NO:16, or an RNA equivalent or a DNA/RNA chimeric thereof and including from 0 to 9 nucleotide analogs, and
the third detection probe oligomer comprises a target-hybridizing sequence as shown in SEQ ID NO:17, or an RNA equivalent or a DNA/RNA chimeric thereof and including from 0 to 9 nucleotide analogs.

Embodiment 63. The formulation of embodiment 61, wherein the formulation comprises first and second detection probe oligomers, wherein
the first detection probe oligomer comprises a target-hybridizing sequence as shown in SEQ ID NO: 15, or an RNA equivalent or a DNA/RNA chimeric thereof and including from 0 to 9 nucleotide analogs, and
the second detection probe oligomer comprises a target-hybridizing sequence as shown in SEQ ID NO:16, or an RNA equivalent or a DNA/RNA chimeric thereof and including from 0 to 9 nucleotide analogs.

Embodiment 64. The formulation of embodiment 63 wherein the formulation further comprises a third detection probe oligomer.

Embodiment 65. The formulation of any one of embodiments 61 to 64, wherein each detection probe oligomer comprises a detectable label.

Embodiment 66. The formulation of embodiment 65, wherein the detectable label is a chemiluminescent or fluorescent label.

Embodiment 67. The formulation of embodiment 66, wherein the detectable label is a fluorescent label and each detection probe oligomer further comprises a non-fluorescent quencher.

Embodiment 68. The formulation of any one of embodiments 43 to 67, wherein one or more cytosines of at least one of the at least two amplification oligomers are 5-methyl cytosine.

Embodiment 69. The formulation of any one of embodiments 55 to 60, wherein one or more cytosines of the at least one detection probe oligomer are 5-methyl cytosine.

Embodiment 70. The formulation of any one of embodiments 61 to 67, wherein one or more cytosines of at least one of the at least two detection probe oligomers are 5-methyl cytosine.

Embodiment 71. The formulation of any one of embodiments 43 to 70, wherein the formulation further comprises one or more of a bulking agent, a surfactant, a nucleotide triphosphate, an enzyme, and an inorganic salt.

Embodiment 72. The formulation of any one of embodiments 43 to 71, wherein the formulation is a dried composition.

Embodiment 73. A formulation for detecting human adenovirus in a sample, the formulation comprising: at least one detection probe oligomer comprising a target-hybridizing sequence as shown in SEQ ID NO:19, or an RNA equivalent or a DNA/RNA chimeric thereof and including from 0 to 9 nucleotide analogs.

Embodiment 74. The formulation of embodiment 73, wherein the at least one detection probe oligomer target-hybridizing sequence is SEQ ID NO:15, SEQ ID NO: 16, or SEQ ID NO:17, or an RNA equivalent or a DNA/RNA equivalent thereof and including from 0 to 9 nucleotide analogs.

Embodiment 75. The formulation of embodiment 73 or 74, wherein the at least one detection probe oligomer comprises a detectable label.

Embodiment 76. The formulation of embodiment 75, wherein the detectable label is a chemiluminescent or fluorescent label.

Embodiment 77. The formulation of embodiment 76, wherein the detectable label is a fluorescent label and the at least one detection probe oligomer further comprises a non-fluorescent quencher.

Embodiment 78. The formulation of embodiment 73 or 74, wherein the formulation comprises at least two detection probe oligomers.

Embodiment 79. The formulation of embodiment 78, wherein the formulation comprises first, second, and third detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs,
the second detection probe oligomer comprises a target-hybridizing sequence as shown in SEQ ID NO:16, or an RNA equivalent or a DNA/RNA chimeric thereof and including from 0 to 9 nucleotide analogs, and
the third detection probe oligomer comprises a target-hybridizing sequence as shown in SEQ ID NO:17, or an RNA equivalent or a DNA/RNA chimeric thereof and including from 0 to 9 nucleotide analogs.

Embodiment 80. The formulation of embodiment 78, wherein the formulation comprises first and second detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs, and
the second detection probe oligomer comprises a target-hybridizing sequence as shown in SEQ ID NO: 16, or an RNA equivalent or a DNA/RNA chimeric thereof and including from 0 to 9 nucleotide analogs.

Embodiment 81. The formulation of embodiment 80, wherein the formulation further comprises a third detection probe oligomer.

Embodiment 82. The formulation of any one of embodiments 78 to 81, wherein each detection probe oligomer comprises a detectable label.

Embodiment 83. The formulation of embodiment 82, wherein the detectable label is a chemiluminescent or fluorescent label.

Embodiment 84. The formulation of embodiment 83, wherein the detectable label is a fluorescent label and each detection probe oligomer further comprises a non-fluorescent quencher.

Embodiment 85. The formulation of any one of embodiments 73 to 77, wherein one or more cytosines of the at least one detection probe oligomer are 5-methyl cytosine.

Embodiment 86. The formulation of any one of embodiments 78 to 84, wherein one or more cytosines of at least one of the at least two detection probe oligomers are 5-methyl cytosine.

Embodiment 87. The formulation of any one of embodiments 73 to 86, wherein the formulation further comprises one or more of a bulking agent, a surfactant, a nucleotide triphosphate, an enzyme, and an inorganic salt.

Embodiment 88. The formulation of any one of embodiments 73 to 87, wherein the formulation is a dried composition.

Embodiment 89. A kit comprising a dried composition as in embodiment 72 or 88.

Embodiment 90. The kit of embodiment 89, further comprising a reconstitution reagent, wherein the dried composition is in a first vial within the kit and the reconstitution reagent is in a second vial within the kit.

Embodiment 91. The kit of embodiment 89 or 90, wherein the dried composition comprises a bulking agent selected from the group consisting of trehalose, raffinose, mannitol, and a combination thereof.

Embodiment 92. The kit of any one of embodiments 89 to 91, wherein the dried composition comprises a non-linear surfactant.

Embodiment 93. The kit of embodiment 92, wherein the non-linear surfactant is polyoxyethylene sorbitan fatty acid ester digitonin.

Embodiment 94. The kit of any one of embodiments 89 to 93, wherein the reconstitution reagent comprises one or more of magnesium chloride, potassium chloride, and ethyl alcohol.

Embodiment 95. A method for preparing an aqueous reaction mixture for determining the presence or absence of a human adenovirus in a sample, the method comprising the step of combining a dried composition as described in embodiment 72 or 88 with a reconstitution reagent to make an aqueous reaction mixture.

Embodiment 96. The method of embodiment 95, wherein the reconstitution reagent comprises one or more of magnesium chloride, potassium chloride, and ethyl alcohol.

Embodiment 97. The method of embodiment 95, wherein the combining step is performed using an automated system comprising a first holder for the dried composition, a second holder for the reconstitution reagent, a programmable controller, and a pipetting device in communication with the programmable controller, and wherein the programmable controller is configured by software instructions to cause the pipetting device to transfer an aliquot of the reconstitution reagent from the second holder location to the first holder location thereby making the aqueous reaction mixture.

Embodiment 98. A method for determining the presence or absence of human adenovirus in a sample, the method comprising:
(1) contacting a sample, said sample suspected of containing human adenovirus, with at least two oligomers capable of amplifying a target region of a human adenovirus target nucleic acid, said oligomer combination comprising
   (a) at least one amplification oligomer comprising a target-hybridizing sequence as shown in SEQ ID NO: 18, or an RNA equivalent or a DNA/RNA chimeric thereof and including from 0 to 9 nucleotide analogs; and
   (b) at least one amplification oligomer selected from the group consisting of
      (i) an oligomer comprising a target-hybridizing sequence as shown in SEQ ID NO:21, or an RNA equivalent or a DNA/RNA chimeric thereof and including from 0 to 9 nucleotide analogs, and
      (ii) an oligomer comprising a target-hybridizing sequence as shown in SEQ ID NO:20, or an RNA equivalent or a DNA/RNA chimeric thereof and including from 0 to 9 nucleotide analogs;
(2) performing an in vitro nucleic acid amplification reaction, wherein any human adenovirus target nucleic acid present in the sample is used as a template for generating an amplification product; and
(3) detecting the presence or absence of the amplification product, thereby determining the presence or absence of human adenovirus in the sample.

Embodiment 99. The method of embodiment 98, wherein the target-hybridizing sequence of (a) is SEQ ID NO:3, SEQ ID NO:4, or SEQ ID NO:5, or an RNA equivalent or a DNA/RNA equivalent thereof and including from 0 to 9 nucleotide analogs.

Embodiment 100. The method of any one of embodiment 98 or 99, wherein the at least two oligomers capable of amplifying the human adenovirus target region comprise at least two amplification oligomers as in (a).

Embodiment 101. The method of embodiment 100, wherein the at least two oligomers capable of amplifying the human adenovirus target region comprise first, second, and third amplification oligomers as in (a), wherein
the first amplification oligomer as in (a) comprises a target-hybridizing sequence as shown in SEQ ID NO:3, or an RNA equivalent or a DNA/RNA chimeric thereof and including from 0 to 9 nucleotide analogs,
the second amplification oligomer as in (a) comprises a target-hybridizing sequence as shown in SEQ ID NO:4, or an RNA equivalent or a DNA/RNA chimeric thereof and including from 0 to 9 nucleotide analogs, and
the third amplification oligomer as in (a) comprises a target-hybridizing sequence as shown in SEQ ID NO:5, or an RNA equivalent or a DNA/RNA chimeric thereof and including from 0 to 9 nucleotide analogs.

Embodiment 102. The method of any one of embodiments 98 to 101, wherein the at least two oligomers capable of amplifying the human adenovirus target region comprise the at least one amplification oligomer of (b)(i).

Embodiment 103. The method of embodiment 102, wherein the target-hybridizing sequence of (b)(i) is SEQ ID NO:6, SEQ ID NO:7, SEQ ID NO:8, or SEQ ID NO:9, or an RNA equivalent or a DNA/RNA equivalent thereof and including from 0 to 9 nucleotide analogs.

Embodiment 104. The method of any one of embodiments 98 to 103, wherein the at least two oligomers capable of amplifying the human adenovirus target region comprise at least two amplification oligomers as in (b)(i).

Embodiment 105. The method of embodiment 104, wherein the at least two oligomers capable of amplifying the human adenovirus target region comprise first, second, third, and fourth amplification oligomers as in (b)(i), wherein
the first amplification oligomer as in (b)(i) comprises a target-hybridizing sequence as shown in SEQ ID NO:6, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:7, or an RNA equivalent or a DNA/RNA chimeric thereof and including from 0 to 9 nucleotide analogs,
the third amplification oligomer as in (b)(i) comprises a target-hybridizing sequence as shown in SEQ ID NO:8, or an RNA equivalent or a DNA/RNA chimeric thereof and including from 0 to 9 nucleotide analogs, and
the fourth amplification oligomer as in (b)(i) comprises a target-hybridizing sequence as shown in SEQ ID NO:9, or an RNA equivalent or a DNA/RNA chimeric thereof and including from 0 to 9 nucleotide analogs.

Embodiment 106. The method of any one of embodiments 98 to 101, wherein the at least two oligomers capable of amplifying the human adenovirus target region comprise the at least one amplification oligomer of (b)(ii).

Embodiment 107. The method of embodiment 106, wherein the target-hybridizing sequence of (b)(ii) is SEQ ID NO:10, SEQ ID NO:11, SEQ ID NO:12, or SEQ ID NO:13, or an RNA equivalent or a DNA/RNA equivalent thereof and including from 0 to 9 nucleotide analogs.

Embodiment 108. The method of any one of embodiments 98 to 107, wherein the at least two oligomers capable of amplifying the human adenovirus target region comprise at least two amplification oligomers as in (b)(ii).

Embodiment 109. The method of embodiment 108, wherein the at least two oligomers capable of amplifying the human adenovirus target region comprise first, second, third, and fourth amplification oligomers as in (b)(ii), wherein
the first amplification oligomer as in (b)(ii) comprises a target-hybridizing sequence as shown in SEQ ID NO: 10, or an RNA equivalent or a DNA/RNA chimeric thereof and including from 0 to 9 nucleotide analogs,
the second amplification oligomer as in (b)(ii) comprises a target-hybridizing sequence as shown in SEQ ID NO:11, or an RNA equivalent or a DNA/RNA chimeric thereof and including from 0 to 9 nucleotide analogs,
the third amplification oligomer as in (b)(ii) comprises a target-hybridizing sequence as shown in SEQ ID NO:12, or an RNA equivalent or a DNA/RNA chimeric thereof and including from 0 to 9 nucleotide analogs, and
the fourth amplification oligomer as in (b)(ii) comprises a target-hybridizing sequence as shown in SEQ ID NO:13, or an RNA equivalent or a DNA/RNA chimeric thereof and including from 0 to 9 nucleotide analogs.

Embodiment 110. The method of any one of embodiments 98 to 109, wherein the detecting step (3) comprises contacting the in vitro nucleic acid amplification reaction with at least one detection probe oligomer configured to specifically hybridize to the amplification product under conditions whereby the presence or absence of the amplification product is determined, thereby determining the presence or absence of human adenovirus in the sample.

Embodiment 111. The method of embodiment 110, wherein the at least one detection probe oligomer target-hybridizing sequence is SEQ ID NO: 15, SEQ ID NO:16, or SEQ ID NO: 17, or an RNA equivalent or a DNA/RNA equivalent thereof and including from 0 to 9 nucleotide analogs.

Embodiment 112. The method of embodiment 110 or 111, wherein the at least one detection probe oligomer comprises a detectable label.

Embodiment 113. The method of embodiment 112, wherein the detectable label is a chemiluminescent or fluorescent label.

Embodiment 114. The method of embodiment 113, wherein the detectable label is a fluorescent label and the at least one detection probe oligomer further comprises a non-fluorescent quencher.

Embodiment 115. The method of embodiment 110 or 111, wherein the detecting step comprises contacting the in vitro nucleic acid amplification reaction with at least two detection probe oligomers.

Embodiment 116. The method of embodiment 115, wherein the detecting step comprises contacting the in vitro nucleic acid amplification reaction with first, second, and third detection probe oligomers, wherein
the first detection probe oligomer comprises a target-hybridizing sequence as shown in SEQ ID NO:15, or an RNA equivalent or a DNA/RNA chimeric thereof and including from 0 to 9 nucleotide analogs,
the second detection probe oligomer comprises a target-hybridizing sequence as shown in SEQ ID NO:16, or an RNA equivalent or a DNA/RNA chimeric thereof and including from 0 to 9 nucleotide analogs, and
the third detection probe oligomer comprises a target-hybridizing sequence as shown in SEQ ID NO:17, or an RNA equivalent or a DNA/RNA chimeric thereof and including from 0 to 9 nucleotide analogs.

Embodiment 117. The method of embodiment 115, wherein the detecting step comprises contacting the in vitro nucleic acid amplification reaction with first and second detection probe oligomers, wherein
the first detection probe oligomer comprises a target-hybridizing sequence as shown in SEQ ID NO: 15, or an RNA equivalent or a DNA/RNA chimeric thereof and including from 0 to 9 nucleotide analogs, and
the second detection probe oligomer comprises a target-hybridizing sequence as shown in SEQ ID NO:16, or an RNA equivalent or a DNA/RNA chimeric thereof and including from 0 to 9 nucleotide analogs.

Embodiment 118. The method of embodiment 117, wherein the detecting step further comprises contacting the in vitro nucleic acid amplification reaction with a third detection probe oligomer.

Embodiment 119. The method of any one of embodiments 115 to 118, wherein each detection probe oligomer comprises a detectable label.

Embodiment 120. The method of embodiment 119, wherein the detectable label is a chemiluminescent or fluorescent label.

Embodiment 121. The method of embodiment 120, wherein the detectable label is a fluorescent label and each detection probe oligomer further comprises a non-fluorescent quencher.

Embodiment 122. The method of any one of embodiments 98 to 121, wherein one or more cytosines of at least one of the at least two amplification oligomers are 5-methyl cytosine.

Embodiment 123. The method of any one of embodiments 110 to 114, wherein one or more cytosines of the at least one detection probe oligomer are 5-methyl cytosine.

Embodiment 124. The method of any one of embodiments 115 to 121, wherein one or more cytosines of at least one of the at least two detection probe oligomers are 5-methyl cytosine.

Embodiment 125. The method of any one of embodiments 98 to 124, wherein the human adenovirus target nucleic acid is separated away from other components of the sample prior to the amplification step (2).

Embodiment 126. The method of embodiment 125, wherein the human adenovirus target nucleic acid is separated away from other components of the sample prior to the contacting step (1).

Embodiment 127. The method of embodiment 125 or 126, wherein the separating step is performed by directly or indirectly binding the human adenovirus target nucleic acid to a solid support to form a [human adenovirus target nucleic acid]:[solid support complex] followed by washing the [human adenovirus target nucleic acid]:[solid support complex] to remove other components of the sample.

Embodiment 128. The method of embodiment 127, wherein the human adenovirus target nucleic acid is hybridized to a target capture oligonucleotide, and wherein the target capture oligonucleotide directly binds to the solid support.

Embodiment 129. The method of embodiment 127, wherein the human adenovirus target nucleic acid is hybridized to a target capture oligonucleotide, and wherein the target capture oligonucleotide directly binds an immobilized probe that is attached to the solid support.

Embodiment 130. The method of any one of embodiments 98 to 129, wherein the in vitro nucleic acid amplification reaction is a PCR reaction.

Embodiment 131. The method of any one of embodiments 98 to 129, wherein the in vitro nucleic acid amplification reaction is an isothermal amplification reaction.

Embodiment 132. The method of embodiment 131, wherein the isothermal amplification reaction is a TMA reaction or a NASBA reaction.

Embodiment 133. The method of any one of embodiments 98 to 132, wherein the in vitro nucleic acid amplification reaction is a quantitative reaction.

Embodiment 134. The method of any one of embodiments 98 to 133, wherein the in vitro nucleic acid amplification reaction is a real-time reaction.

Embodiment 135. The method of any one of embodiments 98 to 134, wherein the sample is a pooled sample.

Embodiment 136. An oligonucleotide comprising a human adenovirus (HAdV)-specific target-hybridizing sequence as shown in SEQ ID NO:18, SEQ ID NO:21, SEQ ID NO:20, or SEQ ID NO:19, or an RNA equivalent or a DNA/RNA chimeric thereof and including from 0 to 9 nucleotide analogs, wherein the 3′ end of said oligonucleotide is attached to a solid support.

Embodiment 137. The oligonucleotide of embodiment 136, wherein the HAdV-specific target-hybridizing sequence is SEQ ID NO:3, SEQ ID NO:4, SEQ ID NO:5, SEQ ID NO:6. SEQ ID NO:7, SEQ ID NO:8, SEQ ID NO:9, SEQ ID NO:10, SEQ ID NO:11, SEQ ID NO: 12, SEQ ID NO:13, SEQ ID NO:15, SEQ ID NO:16, or SEQ ID NO:17, or an RNA equivalent or a DNA/RNA chimeric thereof and including from 0 to 9 nucleotide analogs.

Embodiment 138. A method of chemically synthesizing an amplification oligomer, the method comprising: (a) obtaining a solid support comprising a T nucleotide residue, wherein the T nucleotide residue is covalently bound at its 3′ position to the solid support; (b) coupling a 5′ position of the T nucleotide residue in step (a) to a 3′ position of a G nucleotide residue, thereby generating an oligonucleotide coupled to a solid support and comprising 2 contiguous nucleotide residues; and (c) coupling sequentially fifteen additional nucleotide residues to a terminal 5′ position of the oligonucleotide generated in step (b), wherein at each coupling iteration each additional nucleotide residue provides a 5′ terminal position for coupling with a 3′ position of a next nucleotide residue, so as to generate an oligonucleotide coupled to a solid support and comprising SEQ ID NO:18.

Embodiment 139. The method of embodiment 138, further comprising (d) cleaving the oligonucleotide generated in step (c) from the solid support, wherein the cleaved oligonucleotide comprises a nucleotide sequence consisting of SEQ ID NO: 18.

Embodiment 140. A method of chemically synthesizing an amplification oligomer, the method comprising: (a) obtaining a solid support comprising a C nucleotide residue, wherein the C nucleotide residue is covalently bound at its 3′ position to the solid support; (b) coupling a 5′ position of the C nucleotide residue in step (a) to a 3′ position of a T nucleotide residue, thereby generating an oligonucleotide coupled to a solid support and comprising 2 contiguous nucleotide residues; and (c) coupling sequentially thirteen additional nucleotide residues to a terminal 5′ position of the oligonucleotide generated in step (b), wherein at each coupling iteration each additional nucleotide residue provides a 5′ terminal position for coupling with a 3′ position of a next nucleotide residue, so as to generate an oligonucleotide coupled to a solid support and comprising SEQ ID NO:20.

Embodiment 141. The method of embodiment 140, further comprising (d) cleaving the oligonucleotide generated in step (c) from the solid support, wherein the cleaved oligonucleotide comprises a nucleotide sequence consisting of SEQ ID NO:20.

Embodiment 142. A method of chemically synthesizing an amplification oligomer, the method comprising: (a) obtaining a solid support comprising a G nucleotide residue, wherein the G nucleotide residue is covalently bound at its 3′ position to the solid support; (b) coupling a 5′ position of the G nucleotide residue in step (a) to a 3′ position of a G nucleotide residue, thereby generating an oligonucleotide coupled to a solid support and comprising 2 contiguous nucleotide residues; and (c) coupling sequentially fifteen additional nucleotide residues to a terminal 5′ position of the oligonucleotide generated in step (b), wherein at each coupling iteration each additional nucleotide residue provides a 5′ terminal position for coupling with a 3′ position of a next nucleotide residue, so as to generate an oligonucleotide coupled to a solid support and comprising SEQ ID NO:21.

Embodiment 143. The method of embodiment 142, further comprising (d) cleaving the oligonucleotide generated in step (c) from the solid support, wherein the cleaved oligonucleotide comprises a nucleotide sequence consisting of SEQ ID NO:21.

Embodiment 144. The method of any one of embodiments 138, 140, and 142, wherein one or more of the coupled nucleotide residues is a nucleotide analog.

Embodiment 145. The method of any one of embodiments 138, 140, and 142, wherein one or more of the coupled nucleotide residues is a 5-methyl cytosine.

Embodiment 146. The composition or kit of any one of embodiments 1 to 25, wherein one or more cytosines of at least one of the at least two amplification oligomers are propyne dC.

Embodiment 147. The composition or kit of any one of embodiments 1 to 25, wherein one or more thymines of at least one of the at least two amplification oligomers are propyne dU.

Embodiment 148. The formulation of any one of embodiments 43 to 67, wherein one or more cytosines of at least one of the at least two amplification oligomers are propyne dC.

Embodiment 149. The formulation of any one of embodiments 43 to 67, wherein one or more thymines of at least one of the at least two amplification oligomers are propyne dU.

Embodiment 150. The method of any one of embodiments 98 to 121, wherein one or more cytosines of at least one of the at least two amplification oligomers are propyne dC.

Embodiment 151. The method of any one of embodiments 98 to 121, wherein one or more thymines of at least one of the at least two amplification oligomers are propyne dU.

Embodiment 152. A composition or kit for determining the presence or absence of one or more of human adenovirus (HAdV) subtypes A, B, C, D, E, and F in a sample, said composition or kit comprising:
(a) at least two HAdV A amplification oligomers capable of amplifying a target region of an HAdV subtype A target nucleic acid, wherein
   (i) at least one HAdV A amplification oligomer comprises a target-hybridizing sequence as shown in SEQ ID NO: 18 or SEQ ID NO:26, or an RNA equivalent or a DNA/RNA chimeric thereof and including from 0 to 9 nucleotide analogs; and
   (ii) at least one HAdV A amplification oligomer comprises a target-hybridizing sequence as shown in SEQ ID NO:21, SEQ ID NO:20, SEQ ID NO:27, or SEQ ID NO:28, or an RNA equivalent or a DNA/RNA chimeric thereof and including from 0 to 9 nucleotide analogs; and/or
(b) at least two HAdV B amplification oligomers capable of amplifying a target region of an HAdV subtype B target nucleic acid, wherein
   (i) at least one HAdV B amplification oligomer comprises a target-hybridizing sequence as shown in SEQ ID NO:18, or an RNA equivalent or a DNA/RNA chimeric thereof and including from 0 to 9 nucleotide analogs; and
   (ii) at least one HAdV B amplification oligomer comprises a target-hybridizing sequence as shown in SEQ ID NO:21, SEQ ID NO:20, SEQ ID NO:31, or SEQ ID NO:32, or an RNA equivalent or a DNA/RNA chimeric thereof and including from 0 to 9 nucleotide analogs; and/or
(c) at least two HAdV C amplification oligomers capable of amplifying a target region of an HAdV subtype C target nucleic acid, wherein
   (i) at least one HAdV C amplification oligomer comprises a target-hybridizing sequence as shown in SEQ ID NO: 18 or SEQ ID NO:33, or an RNA equivalent or a DNA/RNA chimeric thereof and including from 0 to 9 nucleotide analogs; and
   (ii) at least one HAdV A amplification oligomer comprises a target-hybridizing sequence as shown in SEQ ID NO:21, SEQ ID NO:20, SEQ ID NO:34, or SEQ ID NO:35, or an RNA equivalent or a DNA/RNA chimeric thereof and including from 0 to 9 nucleotide analogs; and/or
(d) at least two HAdV D amplification oligomers capable of amplifying a target region of an HAdV subtype D target nucleic acid, wherein
   (i) at least one HAdV D amplification oligomer comprises a target-hybridizing sequence as shown in SEQ ID NO:18, or an RNA equivalent or a DNA/RNA chimeric thereof and including from 0 to 9 nucleotide analogs; and
   (ii) at least one HAdV D amplification oligomer comprises a target-hybridizing sequence as shown in SEQ ID NO:21, SEQ ID NO:20, or SEQ ID NO:37, or an RNA equivalent or a DNA/RNA chimeric thereof and including from 0 to 9 nucleotide analogs; and/or
(e) at least two HAdV E amplification oligomers capable of amplifying a target region of an HAdV subtype E target nucleic acid, wherein
   (i) at least one HAdV E amplification oligomer comprises a target-hybridizing sequence as shown in SEQ ID NO: 18 or SEQ ID NO:38, or an RNA equivalent or a DNA/RNA chimeric thereof and including from 0 to 9 nucleotide analogs; and
   (ii) at least one HAdV E amplification oligomer comprises a target-hybridizing sequence as shown in SEQ ID NO:21, SEQ ID NO:20, SEQ ID NO:39, or SEQ ID NO:40, or an RNA equivalent or a DNA/RNA chimeric thereof and including from 0 to 9 nucleotide analogs; and/or
(f) at least two HAdV F amplification oligomers capable of amplifying a target region of an HAdV subtype F target nucleic acid, wherein
   (i) at least one HAdV F amplification oligomer comprises a target-hybridizing sequence as shown in SEQ ID NO:18, SEQ ID NO:42, or SEQ ID NO:43, or an RNA equivalent or a DNA/RNA chimeric thereof and including from 0 to 9 nucleotide analogs; and
   (ii) at least one HAdV F amplification oligomer comprises a target-hybridizing sequence as shown in SEQ ID NO:21, SEQ ID NO:20, SEQ ID NO:44, or SEQ ID NO:45, or an RNA equivalent or a DNA/RNA chimeric thereof and including from 0 to 9 nucleotide analogs.

Embodiment 153. The composition or kit of embodiment 152, wherein the target-hybridizing sequence of (a)(i) is SEQ ID NO:26, or an RNA equivalent or a DNA/RNA chimeric thereof and including from 0 to 9 nucleotide analogs.

Embodiment 154. The composition or kit of embodiment 153, wherein the target-hybridizing sequence of (a)(ii) is SEQ ID NO:27 or SEQ ID NO:28, or an RNA equivalent or a DNA/RNA chimeric thereof and including from 0 to 9 nucleotide analogs.

Embodiment 155. The composition or kit of embodiment 153, wherein the composition or kit comprises first and second amplification oligomers as in (a)(ii), wherein
the first amplification oligomer as in (a)(ii) comprises the target-hybridizing sequence as shown in SEQ ID NO:27, or an RNA equivalent or a DNA/RNA chimeric thereof and including from 0 to 9 nucleotide analogs, and
the second amplification oligomer as in (a)(ii) comprises the target-hybridizing sequence as shown in SEQ ID NO:28, or an RNA equivalent or a DNA/RNA chimeric thereof and including from 0 to 9 nucleotide analogs.

Embodiment 156. The composition or kit of any one of embodiments 152 to 155, wherein the composition or kit comprises first, second, and third amplification oligomers as in (b)(i), wherein
the first amplification oligomer as in (b)(i) comprises a target-hybridizing sequence as shown in SEQ ID NO:3, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:4, or an RNA equivalent or a DNA/RNA chimeric thereof and including from 0 to 9 nucleotide analogs, and
the third amplification oligomer as in (b)(i) comprises a target-hybridizing sequence as shown in SEQ ID NO:5, or an RNA equivalent or a DNA/RNA chimeric thereof and including from 0 to 9 nucleotide analogs.

Embodiment 157. The composition or kit of any one of embodiments 152 to 156, wherein the target-hybridizing sequence of (b)(ii) is SEQ ID NO:31 or SEQ ID NO:32, or an RNA equivalent or a DNA/RNA chimeric thereof and including from 0 to 9 nucleotide analogs.

Embodiment 158. The composition or kit of any one of embodiments 152 to 156, wherein the composition or kit comprises first and second amplification oligomers as in (b)(ii), wherein
the first amplification oligomer as in (b)(ii) comprises the target-hybridizing sequence as shown in SEQ ID NO:31, or an RNA equivalent or a DNA/RNA chimeric thereof and including from 0 to 9 nucleotide analogs, and
the second amplification oligomer as in (b)(ii) comprises the target-hybridizing sequence as shown in SEQ ID NO:32, or an RNA equivalent or a DNA/RNA chimeric thereof and including from 0 to 9 nucleotide analogs.

Embodiment 159. The composition or kit of any one of embodiments 152 to 158, wherein the target-hybridizing sequence of (c)(i) is SEQ ID NO:33, or an RNA equivalent or a DNA/RNA chimeric thereof and including from 0 to 9 nucleotide analogs.

Embodiment 160. The composition or kit of embodiment 159, wherein the target-hybridizing sequence of (c)(ii) is SEQ ID NO:34 or SEQ ID NO:35, or an RNA equivalent or a DNA/RNA chimeric thereof and including from 0 to 9 nucleotide analogs.

Embodiment 161. The composition or kit of any one of embodiments 152 to 160, wherein the composition or kit comprises first and second amplification oligomers as in (c)(ii), wherein
the first amplification oligomer as in (c)(ii) comprises the target-hybridizing sequence as shown in SEQ ID NO:34, or an RNA equivalent or a DNA/RNA chimeric thereof and including from 0 to 9 nucleotide analogs, and
the second amplification oligomer as in (c)(ii) comprises the target-hybridizing sequence as shown in SEQ ID NO:35, or an RNA equivalent or a DNA/RNA chimeric thereof and including from 0 to 9 nucleotide analogs.

Embodiment 162. The composition or kit of any one of embodiments 152 to 161, wherein the target-hybridizing sequence of (d)(ii) is SEQ ID NO:37, or an RNA equivalent or a DNA/RNA chimeric thereof and including from 0 to 9 nucleotide analogs.

Embodiment 163. The composition or kit of any one of embodiments 152 to 162, wherein the target-hybridizing sequence of (e)(i) is SEQ ID NO:38, or an RNA equivalent or a DNA/RNA chimeric thereof and including from 0 to 9 nucleotide analogs.

Embodiment 164. The composition or kit of embodiment 163, wherein the target-hybridizing sequence of (e)(ii) is SEQ ID NO:39 or SEQ ID NO:40, or an RNA equivalent or a DNA/RNA chimeric thereof and including from 0 to 9 nucleotide analogs.

Embodiment 165. The composition or kit of embodiment 163, wherein the composition or kit comprises first and second amplification oligomers as in (e)(ii), wherein
the first amplification oligomer as in (e)(ii) comprises the target-hybridizing sequence as shown in SEQ ID NO:39, or an RNA equivalent or a DNA/RNA chimeric thereof and including from 0 to 9 nucleotide analogs, and
the second amplification oligomer as in (e)(ii) comprises the target-hybridizing sequence as shown in SEQ ID NO:40, or an RNA equivalent or a DNA/RNA chimeric thereof and including from 0 to 9 nucleotide analogs.

Embodiment 166. The composition or kit of any one of embodiments 152 to 165, wherein the target-hybridizing sequence of (f)(i) is SEQ ID NO:42 or SEQ ID NO:43, or an RNA equivalent or a DNA/RNA chimeric thereof and including from 0 to 9 nucleotide analogs.

Embodiment 167. The composition or kit of any one of embodiments 152 to 165, wherein the composition or kit comprises first and second amplification oligomers as in (f)(i), wherein
the first amplification oligomer as in (f)(i) comprises the target-hybridizing sequence as shown in SEQ ID NO:42, or an RNA equivalent or a DNA/RNA chimeric thereof and including from 0 to 9 nucleotide analogs, and
the second amplification oligomer as in (f)(i) comprises the target-hybridizing sequence as shown in SEQ ID NO:43, or an RNA equivalent or a DNA/RNA chimeric thereof and including from 0 to 9 nucleotide analogs.

Embodiment 168. The composition or kit of embodiment 166 or 167, wherein the target-hybridizing sequence of (f)(ii) is SEQ ID NO:44 or SEQ ID NO:45, or an RNA equivalent or a DNA/RNA chimeric thereof and including from 0 to 9 nucleotide analogs.

Embodiment 169. The composition or kit of embodiment 166 or 167, wherein the composition or kit comprises first and second amplification oligomers as in (f)(ii), wherein
the first amplification oligomer as in (f)(ii) comprises the target-hybridizing sequence as shown in SEQ ID NO:44, or an RNA equivalent or a DNA/RNA chimeric thereof and including from 0 to 9 nucleotide analogs, and
the second amplification oligomer as in (f)(ii) comprises the target-hybridizing sequence as shown in SEQ ID NO:45, or an RNA equivalent or a DNA/RNA chimeric thereof and including from 0 to 9 nucleotide analogs.

Embodiment 170. The composition or kit of any one of embodiments 152 to 169, further comprising at least one detection probe oligomer comprising a target-hybridizing sequence configured to specifically hybridize to an amplification product amplifiable by the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers.

Embodiment 171. The composition or kit of embodiment 170, wherein
(a) if the composition or kit comprises the at least two HAdV A amplification oligomers, then the composition or kit further comprises at least one detection probe oligomer comprising a target-hybridizing sequence as shown in SEQ ID NO: 19, SEQ ID NO:29, or SEQ ID NO:30, or an RNA equivalent or a DNA/RNA chimeric thereof and including from 0 to 9 nucleotide analogs;
(b) if the composition or kit comprises the at least two HAdV B amplification oligomers, then the composition or kit further comprises at least one detection probe oligomer comprising a target-hybridizing sequence as shown in SEQ ID NO: 19, or an RNA equivalent or a DNA/RNA chimeric thereof and including from 0 to 9 nucleotide analogs;
(c) if the composition or kit comprises the at least two HAdV C amplification oligomers, then the composition or kit further comprises at least one detection probe oligomer comprising a target-hybridizing sequence as shown in SEQ ID NO:19 or SEQ ID NO:36, or an RNA equivalent or a DNA/RNA chimeric thereof and including from 0 to 9 nucleotide analogs;
(d) if the composition or kit comprises the at least two HAdV D amplification oligomers, then the composition or kit further comprises at least one detection probe oligomer comprising a target-hybridizing sequence as shown in SEQ ID NO:19, or an RNA equivalent or a DNA/RNA chimeric thereof and including from 0 to 9 nucleotide analogs;
(e) if the composition or kit comprises the at least two HAdV E amplification oligomers, then the composition or kit further comprises at least one detection probe oligomer comprising a target-hybridizing sequence as shown in SEQ ID NO: 19 or SEQ ID NO:41, or an RNA equivalent or a DNA/RNA chimeric thereof and including from 0 to 9 nucleotide analogs;
(f) if the composition or kit comprises the at least two HAdV F amplification oligomers, then the composition or kit further comprises at least one detection probe oligomer comprising a target-hybridizing sequence as shown in SEQ ID NO:19 or SEQ ID NO:46, or an RNA equivalent or a DNA/RNA chimeric thereof and including from 0 to 9 nucleotide analogs.

Embodiment 172. The composition or kit of embodiment 171, wherein the at least two HAdV A amplification oligomers comprise (i) a first HAdV A amplification oligomer comprising a target-hybridizing sequence as shown in SEQ ID NO:26, or an RNA equivalent or a DNA/RNA chimeric thereof and including from 0 to 9 nucleotide analogs, and (ii) a second HAdV A amplification oligomer comprising a target-hybridizing sequence as shown in SEQ ID NO:27 or SEQ ID NO:28, or an RNA equivalent or a DNA/RNA chimeric thereof and including from 0 to 9 nucleotide analogs; and wherein the at least one detection probe oligomer of (a) comprises a target-hybridizing sequence as shown in SEQ ID NO:29 or SEQ ID NO:30, or an RNA equivalent or a DNA/RNA chimeric thereof and including from 0 to 9 nucleotide analogs.

Embodiment 173. The composition or kit of embodiment 172, wherein the composition or kit comprises (i) a first detection probe oligomer comprising the target-hybridizing sequence as shown in SEQ ID NO:29, or an RNA equivalent or a DNA/RNA chimeric thereof and including from 0 to 9 nucleotide analogs, and (ii) a second detection probe oligomer comprising the target-hybridizing sequence as shown in SEQ ID NO:30, or an RNA equivalent or a DNA/RNA chimeric thereof and including from 0 to 9 nucleotide analogs.

Embodiment 174. The composition or kit of embodiment 171, wherein the at least two HAdV C amplification oligomers comprise (i) a first HAdV C amplification oligomer comprising a target-hybridizing sequence as shown in SEQ ID NO:33, or an RNA equivalent or a DNA/RNA chimeric thereof and including from 0 to 9 nucleotide analogs, and (ii) a second HAdV C amplification oligomer comprising a target-hybridizing sequence as shown in SEQ ID NO:34 or SEQ ID NO:35, or an RNA equivalent or a DNA/RNA chimeric thereof and including from 0 to 9 nucleotide analogs; and wherein the at least one detection probe oligomer of (c) comprises a target-hybridizing sequence as shown in SEQ ID NO:36, or an RNA equivalent or a DNA/RNA chimeric thereof and including from 0 to 9 nucleotide analogs.

Embodiment 175. The composition or kit of embodiment 171, wherein the at least two HAdV E amplification oligomers comprise (i) a first HAdV E amplification oligomer comprising a target-hybridizing sequence as shown in SEQ ID NO:38, or an RNA equivalent or a DNA/RNA chimeric thereof and including from 0 to 9 nucleotide analogs, and (ii) a second HAdV E amplification oligomer comprising a target-hybridizing sequence as shown in SEQ ID NO:39 or SEQ ID NO:40, or an RNA equivalent or a DNA/RNA chimeric thereof and including from 0 to 9 nucleotide analogs; and wherein the at least one detection probe oligomer of (e) comprises a target-hybridizing sequence as shown in SEQ ID NO:41, or an RNA equivalent or a DNA/RNA chimeric thereof and including from 0 to 9 nucleotide analogs.

Embodiment 176. The composition or kit of embodiment 171, wherein the at least two HAdV F amplification oligomers comprise (i) a first HAdV F amplification oligomer comprising a target-hybridizing sequence as shown in SEQ ID NO:42 or SEQ ID NO:43, or an RNA equivalent or a DNA/RNA chimeric thereof and including from 0 to 9 nucleotide analogs, and (ii) a second HAdV F amplification oligomer comprising a target-hybridizing sequence as shown in SEQ ID NO:44 or SEQ ID NO:45, or an RNA equivalent or a DNA/RNA chimeric thereof and including from 0 to 9 nucleotide analogs; and wherein the at least one detection probe oligomer of (f) comprises a target-hybridizing sequence as shown in SEQ ID NO:46, or an RNA equivalent or a DNA/RNA chimeric thereof and including from 0 to 9 nucleotide analogs.

Embodiment 177. The composition or kit of any one of embodiments 170 to 176, wherein the at least one detection probe oligomer comprises a detectable label.

Embodiment 178. The composition or kit of embodiment 177, wherein the detectable label is a chemiluminescent or fluorescent label.

Embodiment 179. The composition or kit of embodiment 178, wherein the detectable label is a fluorescent label and the at least one detection probe oligomer further comprises a non-fluorescent quencher.

Embodiment 180. The composition or kit of any one of embodiments 152 to 179, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are 5-methyl cytosine.

Embodiment 181. The composition or kit of any one of embodiments 152 to 179, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dC.

Embodiment 182. The composition or kit of any one of embodiments 152 to 179, wherein one or more thymines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dU.

Embodiment 183. The composition or kit of any one of embodiments 170 to 179, wherein one or more cytosines of the at least one detection probe oligomer are 5-methyl cytosine.

Embodiment 184. A composition or kit for detecting human adenovirus in a sample, said composition or kit comprising: at least one detection probe oligomer comprising a target-hybridizing sequence as shown in SEQ ID NO: 19, SEQ ID NO:29, SEQ ID NO:36, SEQ ID NO:41, SEQ ID NO:46, and SEQ ID NO:47, or an RNA equivalent or a DNA/RNA chimeric thereof and including from 0 to 9 nucleotide analogs.

Embodiment 185. The composition or kit of embodiment 184, wherein the at least one detection probe target-hybridizing sequence is SEQ ID NO:47, or an RNA equivalent or a DNA/RNA chimeric thereof and including from 0 to 9 nucleotide analogs.

Embodiment 186. The composition or kit of embodiment 185, wherein the at least one detection probe target-hybridizing sequence is SEQ ID NO:14, SEQ ID NO:15, SEQ ID NO:16, SEQ ID NO:17, or SEQ ID NO:30, or an RNA equivalent or a DNA/RNA chimeric thereof and including from 0 to 9 nucleotide analogs.

Embodiment 187. The composition or kit of any one of embodiments 184 to 186, wherein the at least one detection probe oligomer comprises a detectable label.

Embodiment 188. The composition or kit of embodiment 187, wherein the detectable label is a chemiluminescent or fluorescent label.

Embodiment 189. The composition or kit of embodiment 188, wherein the detectable label is a fluorescent label and the at least one detection probe oligomer further comprises a non-fluorescent quencher.

Embodiment 190. The composition or kit of any one of embodiments 184 to 189, wherein one or more cytosines of the at least one detection probe oligomer are 5-methyl cytosine.

Embodiment 191. A formulation for amplifying a target region of one or more of human adenovirus (HAdV) subtypes A, B, C, D, E, and F in a sample, said formulation comprising:
(1) at least two amplification oligomers comprising
   (a) at least two HAdV A amplification oligomers capable of amplifying a target region of an HAdV subtype A target nucleic acid, wherein
      (i) at least one HAdV A amplification oligomer comprises a target-hybridizing sequence as shown in SEQ ID NO:18 or SEQ ID NO:26, or an RNA equivalent or a DNA/RNA chimeric thereof and including from 0 to 9 nucleotide analogs; and
      (ii) at least one HAdV A amplification oligomer comprises a target-hybridizing sequence as shown in SEQ ID NO:21, SEQ ID NO:20, SEQ ID NO:27 or SEQ ID NO:28, or an RNA equivalent or a DNA/RNA chimeric thereof and including from 0 to 9 nucleotide analogs; and/or
   (b) at least two HAdV B amplification oligomers capable of amplifying a target region of an HAdV subtype B target nucleic acid, wherein
      (i) at least one HAdV B amplification oligomer comprises a target-hybridizing sequence as shown in SEQ ID NO:18, or an RNA equivalent or a DNA/RNA chimeric thereof and including from 0 to 9 nucleotide analogs; and
      (ii) at least one HAdV B amplification oligomer comprises a target-hybridizing sequence as shown in SEQ ID NO:21, SEQ ID NO:20, SEQ ID NO:31 or SEQ ID NO:32, or an RNA equivalent or a DNA/RNA chimeric thereof and including from 0 to 9 nucleotide analogs; and/or
   (c) at least two HAdV C amplification oligomers capable of amplifying a target region of an HAdV subtype C target nucleic acid, wherein
      (i) at least one HAdV C amplification oligomer comprises a target-hybridizing sequence as shown in SEQ ID NO: 18 or SEQ ID NO:33, or an RNA equivalent or a DNA/RNA chimeric thereof and including from 0 to 9 nucleotide analogs; and
      (ii) at least one HAdV A amplification oligomer comprises a target-hybridizing sequence as shown in SEQ ID NO:21, SEQ ID NO:20, SEQ ID NO:34, or SEQ ID NO:35, or an RNA equivalent or a DNA/RNA chimeric thereof and including from 0 to 9 nucleotide analogs; and/or
   (d) at least two HAdV D amplification oligomers capable of amplifying a target region of an HAdV subtype D target nucleic acid, wherein
      (i) at least one HAdV D amplification oligomer comprises a target-hybridizing sequence as shown in SEQ ID NO:18, or an RNA equivalent or a DNA/RNA chimeric thereof and including from 0 to 9 nucleotide analogs; and
      (ii) at least one HAdV D amplification oligomer comprises a target-hybridizing sequence as shown in SEQ ID NO:21, SEQ ID NO:20, or SEQ ID NO:37, or an RNA equivalent or a DNA/RNA chimeric thereof and including from 0 to 9 nucleotide analogs; and/or
   (e) at least two HAdV E amplification oligomers capable of amplifying a target region of an HAdV subtype E target nucleic acid, wherein
      (i) at least one HAdV E amplification oligomer comprises a target-hybridizing sequence as shown in SEQ ID NO: 18 or SEQ ID NO:38, or an RNA equivalent or a DNA/RNA chimeric thereof and including from 0 to 9 nucleotide analogs; and
      (ii) at least one HAdV E amplification oligomer comprises a target-hybridizing sequence as shown in SEQ ID NO:21, SEQ ID NO:20, SEQ ID NO:39, or SEQ ID NO:40, or an RNA equivalent or a DNA/RNA chimeric thereof and including from 0 to 9 nucleotide analogs; and/or
   (f) at least two HAdV F amplification oligomers capable of amplifying a target region of an HAdV subtype F target nucleic acid, wherein
      (i) at least one HAdV F amplification oligomer comprises a target-hybridizing sequence as shown in SEQ ID NO:18, SEQ ID NO:42, or SEQ ID NO:43, or an RNA equivalent or a DNA/RNA chimeric thereof and including from 0 to 9 nucleotide analogs; and
      (ii) at least one HAdV F amplification oligomer comprises a target-hybridizing sequence as shown in SEQ ID NO:21, SEQ ID NO:20, SEQ ID NO:44, or SEQ ID NO:45, or an RNA equivalent or a DNA/RNA chimeric thereof and including from 0 to 9 nucleotide analogs; and
(2) a buffer.

Embodiment 192. The formulation of embodiment 191, wherein the target-hybridizing sequence of (a)(i) is SEQ ID NO:26, or an RNA equivalent or a DNA/RNA chimeric thereof and including from 0 to 9 nucleotide analogs.

Embodiment 193. The formulation of embodiment 192, wherein the target-hybridizing sequence of (a)(ii) is SEQ ID NO:27 or SEQ ID NO:28, or an RNA equivalent or a DNA/RNA chimeric thereof and including from 0 to 9 nucleotide analogs.

Embodiment 194. The formulation of embodiment 192, wherein the formulation comprises first and second amplification oligomers as in (b)(i), wherein
the first amplification oligomer as in (a)(ii) comprises the target-hybridizing sequence as shown in SEQ ID NO:27, or an RNA equivalent or a DNA/RNA chimeric thereof and including from 0 to 9 nucleotide analogs, and
the second amplification oligomer as in (a)(ii) comprises the target-hybridizing sequence as shown in SEQ ID NO:28, or an RNA equivalent or a DNA/RNA chimeric thereof and including from 0 to 9 nucleotide analogs.

Embodiment 195. The formulation of any one of embodiments 191 to 194, wherein the formulation comprises first, second, and third amplification oligomers as in (b)(i), wherein
the first amplification oligomer as in (b)(i) comprises a target-hybridizing sequence as shown in SEQ ID NO:3, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:4, or an RNA equivalent or a DNA/RNA chimeric thereof and including from 0 to 9 nucleotide analogs, and
the third amplification oligomer as in (b)(i) comprises a target-hybridizing sequence as shown in SEQ ID NO:5, or an RNA equivalent or a DNA/RNA chimeric thereof and including from 0 to 9 nucleotide analogs.

Embodiment 196. The formulation of any one of embodiments 191 to 195, wherein the target-hybridizing sequence of (b)(ii) is SEQ ID NO:31 or SEQ ID NO:32, or an RNA equivalent or a DNA/RNA chimeric thereof and including from 0 to 9 nucleotide analogs.

Embodiment 197. The formulation of any one of embodiments 191 to 195, wherein the formulation comprises first and second amplification oligomers as in (b)(ii), wherein
the first amplification oligomer as in (b)(ii) comprises the target-hybridizing sequence as shown in SEQ ID NO:31, or an RNA equivalent or a DNA/RNA chimeric thereof and including from 0 to 9 nucleotide analogs, and
the second amplification oligomer as in (b)(ii) comprises the target-hybridizing sequence as shown in SEQ ID NO:32, or an RNA equivalent or a DNA/RNA chimeric thereof and including from 0 to 9 nucleotide analogs.

Embodiment 198. The formulation of any one of embodiments 191 to 197, wherein the target-hybridizing sequence of (c)(i) is SEQ ID NO:33, or an RNA equivalent or a DNA/RNA chimeric thereof and including from 0 to 9 nucleotide analogs.

Embodiment 199. The formulation of embodiment 198, wherein the target-hybridizing sequence of (c)(ii) is SEQ ID NO:34 or SEQ ID NO:35, or an RNA equivalent or a DNA/RNA chimeric thereof and including from 0 to 9 nucleotide analogs.

Embodiment 200. The formulation of any one of embodiments 191 to 199, wherein the formulation comprises first and second amplification oligomers as in (c)(ii), wherein
the first amplification oligomer as in (c)(ii) comprises the target-hybridizing sequence as shown in SEQ ID NO:34, or an RNA equivalent or a DNA/RNA chimeric thereof and including from 0 to 9 nucleotide analogs, and
the second amplification oligomer as in (c)(ii) comprises the target-hybridizing sequence as shown in SEQ ID NO:35, or an RNA equivalent or a DNA/RNA chimeric thereof and including from 0 to 9 nucleotide analogs.

Embodiment 201. The formulation of any one of embodiments 191 to 200, wherein the target-hybridizing sequence of (d)(ii) is SEQ ID NO:37, or an RNA equivalent or a DNA/RNA chimeric thereof and including from 0 to 9 nucleotide analogs.

Embodiment 202. The formulation of any one of embodiments 191 to 201, wherein the target-hybridizing sequence of (e)(i) is SEQ ID NO:38, or an RNA equivalent or a DNA/RNA chimeric thereof and including from 0 to 9 nucleotide analogs.

Embodiment 203. The formulation of embodiment 202, wherein the target-hybridizing sequence of (e)(ii) is SEQ ID NO:39 or SEQ ID NO:40, or an RNA equivalent or a DNA/RNA chimeric thereof and including from 0 to 9 nucleotide analogs.

Embodiment 204. The formulation of embodiment 202, wherein the formulation comprises first and second amplification oligomers as in (e)(ii), wherein
the first amplification oligomer as in (e)(ii) comprises the target-hybridizing sequence as shown in SEQ ID NO:39, or an RNA equivalent or a DNA/RNA chimeric thereof and including from 0 to 9 nucleotide analogs, and
the second amplification oligomer as in (e)(ii) comprises the target-hybridizing sequence as shown in SEQ ID NO:40, or an RNA equivalent or a DNA/RNA chimeric thereof and including from 0 to 9 nucleotide analogs.

Embodiment 205. The formulation of any one of embodiments 191 to 204, wherein the target-hybridizing sequence of (f)(i) is SEQ ID NO:42 or SEQ ID NO:43, or an RNA equivalent or a DNA/RNA chimeric thereof and including from 0 to 9 nucleotide analogs.

Embodiment 206. The formulation of any one of embodiments 191 to 204, wherein the formulation comprises first and second amplification oligomers as in (f)(i), wherein
the first amplification oligomer as in (f)(i) comprises the target-hybridizing sequence as shown in SEQ ID NO:42, or an RNA equivalent or a DNA/RNA chimeric thereof and including from 0 to 9 nucleotide analogs, and
the second amplification oligomer as in (f)(i) comprises the target-hybridizing sequence as shown in SEQ ID NO:43, or an RNA equivalent or a DNA/RNA chimeric thereof and including from 0 to 9 nucleotide analogs.

Embodiment 207. The formulation of embodiment 205 or 206, wherein the target-hybridizing sequence of (f)(ii) is SEQ ID NO:44 or SEQ ID NO:45, or an RNA equivalent or a DNA/RNA chimeric thereof and including from 0 to 9 nucleotide analogs.

Embodiment 208. The formulation of embodiment 205 or 206, wherein the formulation comprises first and second amplification oligomers as in (f)(ii), wherein
the first amplification oligomer as in (f)(ii) comprises the target-hybridizing sequence as shown in SEQ ID NO:44, or an RNA equivalent or a DNA/RNA chimeric thereof and including from 0 to 9 nucleotide analogs, and
the second amplification oligomer as in (f)(ii) comprises the target-hybridizing sequence as shown in SEQ ID NO:45, or an RNA equivalent or a DNA/RNA chimeric thereof and including from 0 to 9 nucleotide analogs.

Embodiment 209. The formulation of any one of embodiments 191 to 208, further comprising at least one detection probe oligomer comprising a target-hybridizing sequence configured to specifically hybridize to an amplification product amplifiable by the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers.

Embodiment 210. The formulation of embodiment 209, further comprising at, wherein
(a) if the formulation comprises the at least two HAdV A amplification oligomers, then the formulation further comprises at least one detection probe oligomer comprising a target-hybridizing sequence as shown in SEQ ID NO: 19, SEQ ID NO:29, or SEQ ID NO:30, or an RNA equivalent or a DNA/RNA chimeric thereof and including from 0 to 9 nucleotide analogs;
(b) if the formulation comprises the at least two HAdV B amplification oligomers, then the formulation further comprises at least one detection probe oligomer comprising a target-hybridizing sequence as shown in SEQ ID NO: 19, or an RNA equivalent or a DNA/RNA chimeric thereof and including from 0 to 9 nucleotide analogs;
(c) if the formulation comprises the at least two HAdV C amplification oligomers, then the formulation further comprises at least one detection probe oligomer comprising a target-hybridizing sequence as shown in SEQ ID NO: 19 or SEQ ID NO:36, or an RNA equivalent or a DNA/RNA chimeric thereof and including from 0 to 9 nucleotide analogs;
(d) if the formulation comprises the at least two HAdV D amplification oligomers, then the formulation further comprises at least one detection probe oligomer comprising a target-hybridizing sequence as shown in SEQ ID NO: 19, or an RNA equivalent or a DNA/RNA chimeric thereof and including from 0 to 9 nucleotide analogs;
(e) if the formulation comprises the at least two HAdV E amplification oligomers, then the formulation further comprises at least one detection probe oligomer comprising a target-hybridizing sequence as shown in SEQ ID NO: 19 or SEQ ID NO:41, or an RNA equivalent or a DNA/RNA chimeric thereof and including from 0 to 9 nucleotide analogs;
(f) if the formulation comprises the at least two HAdV F amplification oligomers, then the formulation further comprises at least one detection probe oligomer comprising a target-hybridizing sequence as shown in SEQ ID NO: 19 or SEQ ID NO:46, or an RNA equivalent or a DNA/RNA chimeric thereof and including from 0 to 9 nucleotide analogs.

Embodiment 211. The formulation of embodiment 210, wherein the at least two HAdV A amplification oligomers comprise (i) a first HAdV A amplification oligomer comprising a target-hybridizing sequence as shown in SEQ ID NO:26, or an RNA equivalent or a DNA/RNA chimeric thereof and including from 0 to 9 nucleotide analogs, and (ii) a second HAdV A amplification oligomer comprising a target-hybridizing sequence as shown in SEQ ID NO:27 or SEQ ID NO:28, or an RNA equivalent or a DNA/RNA chimeric thereof and including from 0 to 9 nucleotide analogs; and wherein the at least one detection probe oligomer of (a) comprises a target-hybridizing sequence as shown in SEQ ID NO:29 or SEQ ID NO:30, or an RNA equivalent or a DNA/RNA chimeric thereof and including from 0 to 9 nucleotide analogs.

Embodiment 212. The formulation of embodiment 211, wherein the formulation comprises (i) a first detection probe oligomer comprising the target-hybridizing sequence as shown in SEQ ID NO:29, or an RNA equivalent or a DNA/RNA chimeric thereof and including from 0 to 9 nucleotide analogs, and (ii) a second detection probe oligomer comprising the target-hybridizing sequence as shown in SEQ ID NO:30, or an RNA equivalent or a DNA/RNA chimeric thereof and including from 0 to 9 nucleotide analogs.

Embodiment 213. The formulation of embodiment 210, wherein the at least two HAdV C amplification oligomers comprise (i) a first HAdV C amplification oligomer comprising a target-hybridizing sequence as shown in SEQ ID NO:33, or an RNA equivalent or a DNA/RNA chimeric thereof and including from 0 to 9 nucleotide analogs, and (ii) a second HAdV C amplification oligomer comprising a target-hybridizing sequence as shown in SEQ ID NO:34 or SEQ ID NO:35, or an RNA equivalent or a DNA/RNA chimeric thereof and including from 0 to 9 nucleotide analogs; and wherein the at least one detection probe oligomer of (c) comprises a target-hybridizing sequence as shown in SEQ ID NO:36, or an RNA equivalent or a DNA/RNA chimeric thereof and including from 0 to 9 nucleotide analogs.

Embodiment 214. The formulation of embodiment 210, wherein the at least two HAdV E amplification oligomers comprise (i) a first HAdV E amplification oligomer comprising a target-hybridizing sequence as shown in SEQ ID NO:38, or an RNA equivalent or a DNA/RNA chimeric thereof and including from 0 to 9 nucleotide analogs, and (ii) a second HAdV E amplification oligomer comprising a target-hybridizing sequence as shown in SEQ ID NO:39 or SEQ ID NO:40, or an RNA equivalent or a DNA/RNA chimeric thereof and including from 0 to 9 nucleotide analogs; and wherein the at least one detection probe oligomer of (e) comprises a target-hybridizing sequence as shown in SEQ ID NO:41, or an RNA equivalent or a DNA/RNA chimeric thereof and including from 0 to 9 nucleotide analogs.

Embodiment 215. The formulation of embodiment 210, wherein the at least two HAdV F amplification oligomers comprise (i) a first HAdV F amplification oligomer comprising a target-hybridizing sequence as shown in SEQ ID NO:42 or SEQ ID NO:43, or an RNA equivalent or a DNA/RNA chimeric thereof and including from 0 to 9 nucleotide analogs, and (ii) a second HAdV F amplification oligomer comprising a target-hybridizing sequence as shown in SEQ ID NO:44 or SEQ ID NO:45, or an RNA equivalent or a DNA/RNA chimeric thereof and including from 0 to 9 nucleotide analogs; and wherein the at least one detection probe oligomer of (f) comprises a target-hybridizing sequence as shown in SEQ ID NO:46, or an RNA equivalent or a DNA/RNA chimeric thereof and including from 0 to 9 nucleotide analogs.

Embodiment 216. The formulation of any one of embodiments 209 to 215, wherein the at least one detection probe oligomer comprises a detectable label.

Embodiment 217. The formulation of embodiment 216, wherein the detectable label is a chemiluminescent or fluorescent label.

Embodiment 218. The formulation of embodiment 217, wherein the detectable label is a fluorescent label and the at least one detection probe oligomer further comprises a non-fluorescent quencher.

Embodiment 219. The formulation of any one of embodiments 191 to 218, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are 5-methyl cytosine.

Embodiment 220. The formulation of any one of embodiments 191 to 218, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dC.

Embodiment 221. The formulation of any one of embodiments 191 to 218, wherein one or more thymines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dU.

Embodiment 222. The formulation of any one of embodiments 191 to 218, wherein one or more cytosines of the at least one detection probe oligomer are 5-methyl cytosine.

Embodiment 223. The formulation of any one of embodiments 191 to 222, wherein the formulation further comprises one or more of a bulking agent, a surfactant, a nucleotide triphosphate, an enzyme, and an inorganic salt.

Embodiment 224. The formulation of any one of embodiments 191 to 223, wherein the formulation is a dried composition.

Embodiment 225. A formulation for detecting human adenovirus in a sample, the formulation comprising: (a) at least one detection probe oligomer comprising a target-hybridizing sequence as shown in SEQ ID NO: 19, SEQ ID NO:29, SEQ ID NO:36, SEQ ID NO:41, SEQ ID NO:46, or SEQ ID NO:47, or a DNA/RNA chimeric thereof and including from 0 to 9 nucleotide analogs; and (b) a buffer.

Embodiment 226. The formulation of embodiment 225, wherein the at least one detection probe target-hybridizing sequence is SEQ ID NO:47, or an RNA equivalent or a DNA/RNA chimeric thereof and including from 0 to 9 nucleotide analogs.

Embodiment 227. The formulation of embodiment 226, wherein the at least one detection probe target-hybridizing sequence is SEQ ID NO:14, SEQ ID NO:15, SEQ ID NO:16, SEQ ID NO:17, or SEQ ID NO:30, or an RNA equivalent or a DNA/RNA chimeric thereof and including from 0 to 9 nucleotide analogs.

Embodiment 228. The formulation of any one of embodiments 225 to 227, wherein the at least one detection probe oligomer comprises a detectable label.

Embodiment 229. The formulation of embodiment 228, wherein the detectable label is a chemiluminescent or fluorescent label.

Embodiment 230. The formulation of embodiment 229, wherein the detectable label is a fluorescent label and the at least one detection probe oligomer further comprises a non-fluorescent quencher.

Embodiment 231. The formulation of any one of embodiments 225 to 230, wherein one or more cytosines of the at least one detection probe oligomer are 5-methyl cytosine.

Embodiment 232. The formulation of any one of embodiments 225 to 231, wherein the formulation further comprises one or more of a bulking agent, a surfactant, a nucleotide triphosphate, an enzyme, and an inorganic salt.

Embodiment 233. The formulation of any one of embodiments 225 to 232, wherein the formulation is a dried composition.

Embodiment 234. A kit comprising a dried composition as in embodiment 224 or 233.

Embodiment 235. The kit of embodiment 234, further comprising a reconstitution reagent, wherein the dried composition is in a first vial within the kit and the reconstitution reagent is in a second vial within the kit.

Embodiment 236. The kit of embodiment 234 or 235, wherein the dried composition comprises a bulking agent selected from the group consisting of trehalose, raffinose, mannitol, and a combination thereof.

Embodiment 237. The kit of any one of embodiments 234 to 236, wherein the dried composition comprises a non-linear surfactant.

Embodiment 238. The kit of embodiment 237, wherein the non-linear surfactant is polyoxyethylene sorbitan fatty acid ester digitonin.

Embodiment 239. The kit of any one of embodiments 234 to 238, wherein the reconstitution reagent comprises one or more of magnesium chloride, potassium chloride, and ethyl alcohol.

Embodiment 240. A method for preparing an aqueous reaction mixture for determining the presence or absence of a human adenovirus in a sample, the method comprising the step of combining a dried composition as described in embodiment 224 or 233 with a reconstitution reagent to make an aqueous reaction mixture.

Embodiment 241. The method of embodiment 240, wherein the reconstitution reagent comprises one or more of magnesium chloride, potassium chloride, and ethyl alcohol.

Embodiment 242. The method of embodiment 240, wherein the combining step is performed using an automated system comprising a first holder for the dried composition, a second holder for the reconstitution reagent, a programmable controller, and a pipetting device in communication with the programmable controller, and wherein the programmable controller is configured by software instructions to cause the pipetting device to transfer an aliquot of the reconstitution reagent from the second holder location to the first holder location thereby making the aqueous reaction mixture.

Embodiment 243. A method for determining the presence or absence of one or more of human adenovirus (HAdV) subtypes A, B, C, D, E, and F in a sample, the method comprising:
(1) contacting a sample, said sample suspected of containing one or more of HAdV subtypes A, B, C D, E, and F, with at least two oligomers capable of amplifying a target region of an HAdV subtype A, B, C, D, E, and/or F target nucleic acid, said oligomer combination comprising
   (a) at least two HAdV A amplification oligomers capable of amplifying a target region of an HAdV subtype A target nucleic acid, wherein
      (i) at least one HAdV A amplification oligomer comprises a target-hybridizing sequence as shown in SEQ ID NO: 18 or SEQ ID NO:26, or an RNA equivalent or a DNA/RNA chimeric thereof and including from 0 to 9 nucleotide analogs; and
      (ii) at least one HAdV A amplification oligomer comprises a target-hybridizing sequence as shown in SEQ ID NO:21, SEQ ID NO:20, SEQ ID NO:27, or SEQ ID NO:28, or an RNA equivalent or a DNA/RNA chimeric thereof and including from 0 to 9 nucleotide analogs; and/or
   (b) at least two HAdV B amplification oligomers capable of amplifying a target region of an HAdV subtype B target nucleic acid, wherein
      (i) at least one HAdV B amplification oligomer comprises a target-hybridizing sequence as shown in SEQ ID NO:18, or an RNA equivalent or a DNA/RNA chimeric thereof and including from 0 to 9 nucleotide analogs; and
      (ii) at least one HAdV B amplification oligomer comprises a target-hybridizing sequence as shown in SEQ ID NO:21, SEQ ID NO:20, SEQ ID NO:31, or SEQ ID NO:32, or an RNA equivalent or a DNA/RNA chimeric thereof and including from 0 to 9 nucleotide analogs; and/or
   (c) at least two HAdV C amplification oligomers capable of amplifying a target region of an HAdV subtype C target nucleic acid, wherein
      (i) at least one HAdV C amplification oligomer comprises a target-hybridizing sequence as shown in SEQ ID NO:18 or SEQ ID NO:33, or an RNA equivalent or a DNA/RNA chimeric thereof and including from 0 to 9 nucleotide analogs; and
      (ii) at least one HAdV A amplification oligomer comprises a target-hybridizing sequence as shown in SEQ ID NO:21, SEQ ID NO:20, SEQ ID NO:34, or SEQ ID NO:35, or an RNA equivalent or a DNA/RNA chimeric thereof and including from 0 to 9 nucleotide analogs; and/or
   (d) at least two HAdV D amplification oligomers capable of amplifying a target region of an HAdV subtype D target nucleic acid, wherein
      (i) at least one HAdV D amplification oligomer comprises a target-hybridizing sequence as shown in SEQ ID NO: 18, or an RNA equivalent or a DNA/RNA chimeric thereof and including from 0 to 9 nucleotide analogs; and
      (ii) at least one HAdV D amplification oligomer comprises a target-hybridizing sequence as shown in SEQ ID NO:21, SEQ ID NO:20, or SEQ ID NO:37, or an RNA equivalent or a DNA/RNA chimeric thereof and including from 0 to 9 nucleotide analogs; and/or
   (e) at least two HAdV E amplification oligomers capable of amplifying a target region of an HAdV subtype E target nucleic acid, wherein
      (i) at least one HAdV E amplification oligomer comprises a target-hybridizing sequence as shown in SEQ ID NO:18 or SEQ ID NO:38, or an RNA equivalent or a DNA/RNA chimeric thereof and including from 0 to 9 nucleotide analogs; and
      (ii) at least one HAdV E amplification oligomer comprises a target-hybridizing sequence as shown in SEQ ID NO:21, SEQ ID NO:20, SEQ ID NO:39, or SEQ ID NO:40, or an RNA equivalent or a DNA/RNA chimeric thereof and including from 0 to 9 nucleotide analogs; and/or
   (f) at least two HAdV F amplification oligomers capable of amplifying a target region of an HAdV subtype F target nucleic acid, wherein
      (i) at least one HAdV F amplification oligomer comprises a target-hybridizing sequence as shown in SEQ ID NO:18, SEQ ID NO:42, or SEQ ID NO:43, or an RNA equivalent or a DNA/RNA chimeric thereof and including from 0 to 9 nucleotide analogs; and
      (ii) at least one HAdV F amplification oligomer comprises a target-hybridizing sequence as shown in SEQ ID NO:21, SEQ ID NO:20, SEQ ID NO:44, or SEQ ID NO:45, or an RNA equivalent or a DNA/RNA chimeric thereof and including from 0 to 9 nucleotide analogs;
(2) performing an in vitro nucleic acid amplification reaction, wherein any HAdV subtype A, B, C, D, E, and/or F target nucleic acid present in the sample is used as a template for generating an amplification product; and
(3) detecting the presence or absence of the amplification product, thereby determining the presence or absence of one or more of HAdV subtypes A, B, C, D, E, and F in the sample.

Embodiment 244. The method of embodiment 243, wherein the target-hybridizing sequence of (a)(i) is SEQ ID NO:26, or an RNA equivalent or a DNA/RNA chimeric thereof and including from 0 to 9 nucleotide analogs.

Embodiment 245. The method of embodiment 244, wherein the target-hybridizing sequence of (a)(ii) is SEQ ID NO:27 or SEQ ID NO:28, or an RNA equivalent or a DNA/RNA chimeric thereof and including from 0 to 9 nucleotide analogs.

Embodiment 246. The method of embodiment 244, wherein the sample is contacted with first and second amplification oligomers as in (a)(ii), wherein
the first amplification oligomer as in (a)(ii) comprises the target-hybridizing sequence as shown in SEQ ID NO:27, or an RNA equivalent or a DNA/RNA chimeric thereof and including from 0 to 9 nucleotide analogs, and
the second amplification oligomer as in (a)(ii) comprises the target-hybridizing sequence as shown in SEQ ID NO:28, or an RNA equivalent or a DNA/RNA chimeric thereof and including from 0 to 9 nucleotide analogs.

Embodiment 247. The method of any one of embodiments 243 to 246, wherein the sample is contacted with first, second, and third amplification oligomers as in (b)(i), wherein
the first amplification oligomer as in (b)(i) comprises a target-hybridizing sequence as shown in SEQ ID NO:3, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:4, or an RNA equivalent or a DNA/RNA chimeric thereof and including from 0 to 9 nucleotide analogs, and
the third amplification oligomer as in (b)(i) comprises a target-hybridizing sequence as shown in SEQ ID NO:5, or an RNA equivalent or a DNA/RNA chimeric thereof and including from 0 to 9 nucleotide analogs.

Embodiment 248. The method of any one of embodiments 243 to 247, wherein the target-hybridizing sequence of (b)(ii) is SEQ ID NO:31 or SEQ ID NO:32, or an RNA equivalent or a DNA/RNA chimeric thereof and including from 0 to 9 nucleotide analogs.

Embodiment 249. The method of any one of embodiments 243 to 247, wherein the sample is contacted with first and second amplification oligomers as in (b)(ii), wherein
the first amplification oligomer as in (b)(ii) comprises the target-hybridizing sequence as shown in SEQ ID NO:31, or an RNA equivalent or a DNA/RNA chimeric thereof and including from 0 to 9 nucleotide analogs, and
the second amplification oligomer as in (b)(ii) comprises the target-hybridizing sequence as shown in SEQ ID NO:32, or an RNA equivalent or a DNA/RNA chimeric thereof and including from 0 to 9 nucleotide analogs.

Embodiment 250. The method of any one of embodiments 243 to 249, wherein the target-hybridizing sequence of (c)(i) is SEQ ID NO:33, or an RNA equivalent or a DNA/RNA chimeric thereof and including from 0 to 9 nucleotide analogs.

Embodiment 251. The method of embodiment 250, wherein the target-hybridizing sequence of (c)(ii) is SEQ ID NO:34 or SEQ ID NO:35, or an RNA equivalent or a DNA/RNA chimeric thereof and including from 0 to 9 nucleotide analogs.

Embodiment 252. The method of any one of embodiments 243 to 251, wherein the sample is contacted with first and second amplification oligomers as in (c)(ii), wherein
the first amplification oligomer as in (c)(ii) comprises the target-hybridizing sequence as shown in SEQ ID NO:34, or an RNA equivalent or a DNA/RNA chimeric thereof and including from 0 to 9 nucleotide analogs, and
the second amplification oligomer as in (c)(ii) comprises the target-hybridizing sequence as shown in SEQ ID NO:35, or an RNA equivalent or a DNA/RNA chimeric thereof and including from 0 to 9 nucleotide analogs.

Embodiment 253. The method of any one of embodiments 243 to 252, wherein the target-hybridizing sequence of (d)(ii) is SEQ ID NO:37, or an RNA equivalent or a DNA/RNA chimeric thereof and including from 0 to 9 nucleotide analogs.

Embodiment 254. The method of any one of embodiments 243 to 253, wherein the target-hybridizing sequence of (e)(i) is SEQ ID NO:38, or an RNA equivalent or a DNA/RNA chimeric thereof and including from 0 to 9 nucleotide analogs.

Embodiment 255. The method of embodiment 254, wherein the target-hybridizing sequence of (e)(ii) is SEQ ID NO:39 or SEQ ID NO:40, or an RNA equivalent or a DNA/RNA chimeric thereof and including from 0 to 9 nucleotide analogs.

Embodiment 256. The method of embodiment 254, wherein the sample is contacted with first and second amplification oligomers as in (e)(ii), wherein
the first amplification oligomer as in (e)(ii) comprises the target-hybridizing sequence as shown in SEQ ID NO:39, or an RNA equivalent or a DNA/RNA chimeric thereof and including from 0 to 9 nucleotide analogs, and
the second amplification oligomer as in (e)(ii) comprises the target-hybridizing sequence as shown in SEQ ID NO:40, or an RNA equivalent or a DNA/RNA chimeric thereof and including from 0 to 9 nucleotide analogs.

Embodiment 257. The method of any one of embodiments 243 to 256, wherein the target-hybridizing sequence of (f)(i) is SEQ ID NO:42 or SEQ ID NO:43, or an RNA equivalent or a DNA/RNA chimeric thereof and including from 0 to 9 nucleotide analogs.

Embodiment 258. The method of any one of embodiments 243 to 256, wherein the sample is contacted with first and second amplification oligomers as in (f)(i), wherein
the first amplification oligomer as in (f)(i) comprises the target-hybridizing sequence as shown in SEQ ID NO:42, or an RNA equivalent or a DNA/RNA chimeric thereof and including from 0 to 9 nucleotide analogs, and
the second amplification oligomer as in (f)(i) comprises the target-hybridizing sequence as shown in SEQ ID NO:43, or an RNA equivalent or a DNA/RNA chimeric thereof and including from 0 to 9 nucleotide analogs.

Embodiment 259. The method of embodiment 243 to 258, wherein the target-hybridizing sequence of (f)(ii) is SEQ ID NO:44 or SEQ ID NO:45, or an RNA equivalent or a DNA/RNA chimeric thereof and including from 0 to 9 nucleotide analogs.

Embodiment 260. The method of embodiment 257 or 258, wherein the sample is contacted with first and second amplification oligomers as in (f)(ii), wherein
the first amplification oligomer as in (f)(ii) comprises the target-hybridizing sequence as shown in SEQ ID NO:44, or an RNA equivalent or a DNA/RNA chimeric thereof and including from 0 to 9 nucleotide analogs, and
the second amplification oligomer as in (f)(ii) comprises the target-hybridizing sequence as shown in SEQ ID NO:45, or an RNA equivalent or a DNA/RNA chimeric thereof and including from 0 to 9 nucleotide analogs.

Embodiment 261. The method of any one of embodiments 243 to 260, wherein the detecting step (3) comprises contacting the in vitro nucleic acid amplification reaction with at least one detection probe oligomer configured to specifically hybridize to the amplification product under conditions whereby the presence or absence of the amplification product is determined, thereby determining the presence or absence of one or more of HAdV subtypes A, B, C, D, E, and F in the sample.

Embodiment 262. The method of embodiment 261, wherein
(a) if the sample is contacted with the at least two HAdV A amplification oligomers, then the composition or kit further comprises at least one detection probe oligomer comprising a target-hybridizing sequence as shown in SEQ ID NO:19, SEQ ID NO:29, or SEQ ID NO:30, or an RNA equivalent or a DNA/RNA chimeric thereof and including from 0 to 9 nucleotide analogs;
(b) if the sample is contacted with the at least two HAdV B amplification oligomers, then the composition or kit further comprises at least one detection probe oligomer comprising a target-hybridizing sequence as shown in SEQ ID NO:19, or an RNA equivalent or a DNA/RNA chimeric thereof and including from 0 to 9 nucleotide analogs;
(c) if the sample is contacted with the at least two HAdV C amplification oligomers, then the composition or kit further comprises at least one detection probe oligomer comprising a target-hybridizing sequence as shown in SEQ ID NO:19 or SEQ ID NO:36, or an RNA equivalent or a DNA/RNA chimeric thereof and including from 0 to 9 nucleotide analogs;
(d) if the sample is contacted with the at least two HAdV D amplification oligomers, then the composition or kit further comprises at least one detection probe oligomer comprising a target-hybridizing sequence as shown in SEQ ID NO:19, or an RNA equivalent or a DNA/RNA chimeric thereof and including from 0 to 9 nucleotide analogs;
(e) if the sample is contacted with the at least two HAdV E amplification oligomers, then the composition or kit further comprises at least one detection probe oligomer comprising a target-hybridizing sequence as shown in SEQ ID NO:19 or SEQ ID NO:41, or an RNA equivalent or a DNA/RNA chimeric thereof and including from 0 to 9 nucleotide analogs;
(f) if the sample is contacted with the at least two HAdV F amplification oligomers, then the composition or kit further comprises at least one detection probe oligomer comprising a target-hybridizing sequence as shown in SEQ ID NO:19 or SEQ ID NO:46, or an RNA equivalent or a DNA/RNA chimeric thereof and including from 0 to 9 nucleotide analogs.

Embodiment 263. The method of embodiment 262, wherein the at least two HAdV A amplification oligomers comprise (i) a first HAdV A amplification oligomer comprising a target-hybridizing sequence as shown in SEQ ID NO:26, or an RNA equivalent or a DNA/RNA chimeric thereof and including from 0 to 9 nucleotide analogs, and (ii) a second HAdV A amplification oligomer comprising a target-hybridizing sequence as shown in SEQ ID NO:27 or SEQ ID NO:28, or an RNA equivalent or a DNA/RNA chimeric thereof and including from 0 to 9 nucleotide analogs; and wherein the at least one detection probe oligomer of (a) comprises a target-hybridizing sequence as shown in SEQ ID NO:29 or SEQ ID NO:30, or an RNA equivalent or a DNA/RNA chimeric thereof and including from 0 to 9 nucleotide analogs.

Embodiment 264. The method of embodiment 263, wherein the composition or kit comprises (i) a first detection probe oligomer comprising the target-hybridizing sequence as shown in SEQ ID NO:29, or an RNA equivalent or a DNA/RNA chimeric thereof and including from 0 to 9 nucleotide analogs, and (ii) a second detection probe oligomer comprising the target-hybridizing sequence as shown in SEQ ID NO:30, or an RNA equivalent or a DNA/RNA chimeric thereof and including from 0 to 9 nucleotide analogs.

Embodiment 265. The method of embodiment 262, wherein the at least two HAdV C amplification oligomers comprise (i) a first HAdV C amplification oligomer comprising a target-hybridizing sequence as shown in SEQ ID NO:33, or an RNA equivalent or a DNA/RNA chimeric thereof and including from 0 to 9 nucleotide analogs, and (ii) a second HAdV C amplification oligomer comprising a target-hybridizing sequence as shown in SEQ ID NO:34 or SEQ ID NO:35, or an RNA equivalent or a DNA/RNA chimeric thereof and including from 0 to 9 nucleotide analogs; and wherein the at least one detection probe oligomer of (c) comprises a target-hybridizing sequence as shown in SEQ ID NO:36, or an RNA equivalent or a DNA/RNA chimeric thereof and including from 0 to 9 nucleotide analogs.

Embodiment 266. The method of embodiment 262, wherein the at least two HAdV E amplification oligomers comprise (i) a first HAdV E amplification oligomer comprising a target-hybridizing sequence as shown in SEQ ID NO:38, or an RNA equivalent or a DNA/RNA chimeric thereof and including from 0 to 9 nucleotide analogs, and (ii) a second HAdV E amplification oligomer comprising a target-hybridizing sequence as shown in SEQ ID NO:39 or SEQ ID NO:40, or an RNA equivalent or a DNA/RNA chimeric thereof and including from 0 to 9 nucleotide analogs; and wherein the at least one detection probe oligomer of (e) comprises a target-hybridizing sequence as shown in SEQ ID NO:41, or an RNA equivalent or a DNA/RNA chimeric thereof and including from 0 to 9 nucleotide analogs.

Embodiment 267. The method of embodiment 262, wherein the at least two HAdV F amplification oligomers comprise (i) a first HAdV F amplification oligomer comprising a target-hybridizing sequence as shown in SEQ ID NO:42 or SEQ ID NO:43, or an RNA equivalent or a DNA/RNA chimeric thereof and including from 0 to 9 nucleotide analogs, and (ii) a second HAdV F amplification oligomer comprising a target-hybridizing sequence as shown in SEQ ID NO:44 or SEQ ID NO:45, or an RNA equivalent or a DNA/RNA chimeric thereof and including from 0 to 9 nucleotide analogs; and wherein the at least one detection probe oligomer of (f) comprises a target-hybridizing sequence as shown in SEQ ID NO:46, or an RNA equivalent or a DNA/RNA chimeric thereof and including from 0 to 9 nucleotide analogs.

Embodiment 268. The method of any one of embodiments 261 to 267, wherein the at least one detection probe oligomer comprises a detectable label.

Embodiment 269. The method of embodiment 268, wherein the detectable label is a chemiluminescent or fluorescent label.

Embodiment 270. The method of embodiment 269, wherein the detectable label is a fluorescent label and the at least one detection probe oligomer further comprises a non-fluorescent quencher.

Embodiment 272. The method of any one of embodiments 243 to 270, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are 5-methyl cytosine.

Embodiment 273. The method of any one of embodiments 243 to 270, wherein one or more cytosines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dC.

Embodiment 274. The method of any one of embodiments 243 to 270, wherein one or more thymines of at least one of the at least two HAdV A amplification oligomers, the at least two HAdV B amplification oligomers, the at least two HAdV C amplification oligomers, the at least two HAdV D amplification oligomers, the at least two HAdV E amplification oligomers, and/or the at least two HAdV F amplification oligomers are propyne dU.

Embodiment 275. The method of any one of embodiments 261 to 270, wherein one or more cytosines of the at least one detection probe oligomer are 5-methyl cytosine.

Embodiment 276. The method of any one of embodiments 243 to 275, wherein the human adenovirus target nucleic acid is separated away from other components of the sample prior to the amplification step (2).

Embodiment 277. The method of embodiment 276, wherein the human adenovirus target nucleic acid is separated away from other components of the sample prior to the contacting step (1).

Embodiment 278. The method of embodiment 276 or 277, wherein the separating step is performed by directly or indirectly binding the human adenovirus target nucleic acid to a solid support to form a [human adenovirus target nucleic acid]:[solid support complex] followed by washing the [human adenovirus target nucleic acid]:[solid support complex] to remove other components of the sample.

Embodiment 279. The method of embodiment 278, wherein the human adenovirus target nucleic acid is hybridized to a target capture oligonucleotide, and wherein the target capture oligonucleotide directly binds to the solid support.

Embodiment 280. The method of embodiment 278, wherein the human adenovirus target nucleic acid is hybridized to a target capture oligonucleotide, and wherein the target capture oligonucleotide directly binds an immobilized probe that is attached to the solid support.

Embodiment 281. The method of any one of embodiments 243 to 280, wherein the in vitro nucleic acid amplification reaction is a PCR reaction.

Embodiment 282. The method of any one of embodiments 243 to 280, wherein the in vitro nucleic acid amplification reaction is an isothermal amplification reaction.

Embodiment 283. The method of embodiment 282, wherein the isothermal amplification reaction is a TMA reaction or a NASBA reaction.

Embodiment 284. The method of any one of embodiments 243 to 283, wherein the in vitro nucleic acid amplification reaction is a quantitative reaction.

Embodiment 285. The method of any one of embodiments 243 to 284, wherein the in vitro nucleic acid amplification reaction is a real-time reaction.

Embodiment 286. The method of any one of embodiments 243 to 285, wherein the sample is a pooled sample.

Embodiment 287. An oligonucleotide comprising a human adenovirus (HAdV)-specific target-hybridizing sequence as shown in SEQ ID NO:18, SEQ ID NO:21, SEQ ID NO:20, SEQ ID NO: 19, SEQ ID NO:26, SEQ ID NO:27, SEQ ID NO:28, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, or SEQ ID NO:46, or SEQ ID NO:47, or an RNA equivalent or a DNA/RNA chimeric thereof and including from 0 to 9 nucleotide analogs, wherein the 3′ end of said oligonucleotide is attached to a solid support.

Embodiment 288. The oligonucleotide of embodiment 287, wherein the HAdV-specific target-hybridizing sequence is SEQ ID NO:18, SEQ ID NO:20, SEQ ID NO:21, or SEQ ID NO:47.

Embodiment 289. The oligonucleotide of embodiment 288, wherein the HAdV-specific target-hybridizing sequence is SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO:13, SEQ ID NO:15, SEQ ID NO: 16, or SEQ ID NO: 17, or SEQ ID NO:30, or an RNA equivalent or a DNA/RNA chimeric thereof and including from 0 to 9 nucleotide analogs.

Embodiment 290. The composition or kit of embodiment 2, wherein the target-hybridizing sequence of (a) is SEQ ID NO:3, SEQ ID NO:48, SEQ ID NO:49, SEQ ID NO:54, SEQ ID NO:55, or SEQ ID NO:56.

Embodiment 291. The composition or kit of embodiment 6 or 290, wherein the target-hybridizing sequence of (b)(i) is SEQ ID NO:50, SEQ ID NO:51, SEQ ID NO:52, SEQ ID NO:9, SEQ ID NO:57, SEQ ID NO:58, SEQ ID NO:59, or SEQ ID NO:60.

Embodiment 292. The composition or kit of embodiment 2, wherein the target-hybridizing sequence of (a) is SEQ ID NO:3, SEQ ID NO:48, or SEQ ID NO:49.

Embodiment 293. The composition or kit of embodiment 6 or 292, wherein the target-hybridizing sequence of (b)(i) is SEQ ID NO:50, SEQ ID NO:51, SEQ ID NO 52, or SEQ ID NO:9.

Embodiment 294. The composition or kit of embodiment 2, wherein the target-hybridizing sequence of (a) is SEQ ID NO:54, SEQ ID NO:55, or SEQ ID NO:56.

Embodiment 295. The composition or kit of embodiment 6 or 294, wherein the target-hybridizing sequence of (b)(i) is SEQ ID NO:57, SEQ ID NO:58, SEQ ID NO:59, or SEQ ID NO:60.

Embodiment 296. The composition or kit of embodiment 4, wherein
the first amplification oligomer as in (a) comprises the target-hybridizing sequence shown in SEQ ID NO:3,
the second amplification oligomer as in (a) comprises the target-hybridizing sequence shown in SEQ ID NO:48, and
the third amplification oligomer as in (a) comprises the target-hybridizing sequence shown in SEQ ID NO:49.

Embodiment 297. The composition or kit of embodiment 8, wherein
the first amplification oligomer as in (b)(i) comprises the target-hybridizing sequence shown in SEQ ID NO:50,
the second amplification oligomer as in (b)(i) comprises the target-hybridizing sequence shown in SEQ ID NO:51,
the third amplification oligomer as in (b)(i) comprises the target-hybridizing sequence shown in SEQ ID NO:52, and
the fourth amplification oligomer as in (b)(i) comprises the target-hybridizing sequence shown in SEQ ID NO:9.

Embodiment 298. The composition or kit of embodiment 4, wherein
the first amplification oligomer as in (a) comprises the target-hybridizing sequence shown in SEQ ID NO:54,
the second amplification oligomer as in (a) comprises the target-hybridizing sequence shown in SEQ ID NO:55, and
the third amplification oligomer as in (a) comprises the target-hybridizing sequence shown in SEQ ID NO:56.

Embodiment 299. The composition or kit of embodiment 8, wherein
the first amplification oligomer as in (b)(i) comprises the target-hybridizing sequence shown in SEQ ID NO:57,
the second amplification oligomer as in (b)(i) comprises the target-hybridizing sequence shown in SEQ ID NO:58,
the third amplification oligomer as in (b)(i) comprises the target-hybridizing sequence shown in SEQ ID NO:59, and
the fourth amplification oligomer as in (b)(i) comprises the target-hybridizing sequence shown in SEQ ID NO:60.

Embodiment 300. The composition or kit of embodiment 15 or 30, wherein the at least one detection probe oligomer target-hybridizing sequence is SEQ ID NO:53, SEQ ID NO: 16, or SEQ ID NO:17.

Embodiment 301. The composition or kit of embodiment 15 or 30, wherein the at least one detection probe oligomer target-hybridizing sequence is SEQ ID NO:61, SEQ ID NO:62, or SEQ ID NO:63.

Embodiment 302. The composition or kit of embodiment 20 or 35, wherein
the first detection probe oligomer comprises the target-hybridizing sequence shown in SEQ ID NO:53,
the second detection probe oligomer comprises the target-hybridizing sequence shown in SEQ ID NO: 16, and
the third detection probe oligomer comprises the target-hybridizing sequence shown in SEQ ID NO:17.

Embodiment 303. The composition or kit of embodiment 20 or 35, wherein
the first detection probe oligomer comprises the target-hybridizing sequence shown in SEQ ID NO:61,
the second detection probe oligomer comprises the target-hybridizing sequence shown in SEQ ID NO:62, and
the third detection probe oligomer comprises the target-hybridizing sequence shown in SEQ ID NO:63.

Embodiment 304. The composition or kit of embodiment 26, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:57, SEQ ID NO:58, SEQ ID NO:59, or SEQ ID NO:60.

Embodiment 305. The composition or kit of embodiment 27 or 41, wherein the at least one detection probe oligomer target-hybridizing sequence is SEQ ID NO:53, SEQ ID NO:61, SEQ ID NO:62, or SEQ ID NO:63.

Embodiment 306. The composition or kit of embodiment 28 or 42, wherein at least one detection probe oligomer target-hybridizing sequence is SEQ ID NO:53, SEQ ID NO:61, SEQ ID NO:62, or SEQ ID NO:63.

Embodiment 307. The formulation of embodiment 44, wherein the target-hybridizing sequence of (a) is SEQ ID NO:3, SEQ ID NO:48, SEQ ID NO:49, SEQ ID NO:54, SEQ ID NO:55, or SEQ ID NO:56.

Embodiment 308. The formulation of embodiment 48 or 307, wherein the target-hybridizing sequence of (b)(i) is SEQ ID NO:50, SEQ ID NO:51, SEQ ID NO:52, SEQ ID NO:9, SEQ ID NO:57, SEQ ID NO:58, SEQ ID NO:59, or SEQ ID NO:60.

Embodiment 309. The formulation of embodiment 44, wherein the target-hybridizing sequence of (a) is SEQ ID NO:3, SEQ ID NO:48, or SEQ ID NO:49.

Embodiment 310. The formulation of embodiment 48 or 309, wherein the target-hybridizing sequence of (b)(i) is SEQ ID NO:50, SEQ ID NO:51, SEQ ID NO 52, or SEQ ID NO:9.

Embodiment 311. The formulation of embodiment 44, wherein the target-hybridizing sequence of (a) is SEQ ID NO:54, SEQ ID NO:55, or SEQ ID NO:56.

Embodiment 312. The formulation of embodiment 48 or 311, wherein the target-hybridizing sequence of (b)(i) is SEQ ID NO:57, SEQ ID NO:58, SEQ ID NO:59, or SEQ ID NO:60.

Embodiment 313. The formulation of embodiment 46, wherein
the first amplification oligomer as in (a) comprises the target-hybridizing sequence shown in SEQ ID NO:3,
the second amplification oligomer as in (a) comprises the target-hybridizing sequence shown in SEQ ID NO:48, and
the third amplification oligomer as in (a) comprises the target-hybridizing sequence shown in SEQ ID NO:49.

Embodiment 314. The formulation of embodiment 50, wherein
the first amplification oligomer as in (b)(i) comprises the target-hybridizing sequence shown in SEQ ID NO:50,
the second amplification oligomer as in (b)(i) comprises the target-hybridizing sequence shown in SEQ ID NO:51,
the third amplification oligomer as in (b)(i) comprises the target-hybridizing sequence shown in SEQ ID NO:52, and
the fourth amplification oligomer as in (b)(i) comprises the target-hybridizing sequence shown in SEQ ID NO:9.

Embodiment 315. The formulation of embodiment 46, wherein
the first amplification oligomer as in (a) comprises the target-hybridizing sequence shown in SEQ ID NO:54,
the second amplification oligomer as in (a) comprises the target-hybridizing sequence shown in SEQ ID NO:55, and
the third amplification oligomer as in (a) comprises the target-hybridizing sequence shown in SEQ ID NO:56.

Embodiment 316. The formulation of embodiment 50, wherein
the first amplification oligomer as in (b)(i) comprises the target-hybridizing sequence shown in SEQ ID NO:57,
the second amplification oligomer as in (b)(i) comprises the target-hybridizing sequence shown in SEQ ID NO:58,
the third amplification oligomer as in (b)(i) comprises the target-hybridizing sequence shown in SEQ ID NO:59, and
the fourth amplification oligomer as in (b)(i) comprises the target-hybridizing sequence shown in SEQ ID NO:60.

Embodiment 317. The formulation of embodiment 57 or 74, wherein the at least one detection probe oligomer target-hybridizing sequence is SEQ ID NO:53, SEQ ID NO: 16, or SEQ ID NO:17.

Embodiment 318. The formulation of embodiment 57 or 74, wherein the at least one detection probe oligomer target-hybridizing sequence is SEQ ID NO:61, SEQ ID NO:62, or SEQ ID NO:63.

Embodiment 319. The formulation of embodiment 62 or 79, wherein
the first detection probe oligomer comprises the target-hybridizing sequence shown in SEQ ID NO:53,
the second detection probe oligomer comprises the target-hybridizing sequence shown in SEQ ID NO: 16, and
the third detection probe oligomer comprises the target-hybridizing sequence shown in SEQ ID NO:17.

Embodiment 320. The formulation of embodiment 62 or 79, wherein
the first detection probe oligomer comprises the target-hybridizing sequence shown in SEQ ID NO:61,
the second detection probe oligomer comprises the target-hybridizing sequence shown in SEQ ID NO:62, and
the third detection probe oligomer comprises the target-hybridizing sequence shown in SEQ ID NO:63.

Embodiment 321. The formulation of embodiment 68, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:57, SEQ ID NO:58, SEQ ID NO:59, or SEQ ID NO:60.

Embodiment 322. The formulation of embodiment 69 or 85, wherein the at least one detection probe oligomer target-hybridizing sequence is SEQ ID NO:53, SEQ ID NO:61, SEQ ID NO:62, or SEQ ID NO:63.

Embodiment 323. The formulation of embodiment 70 or 86, wherein at least one detection probe oligomer target-hybridizing sequence is SEQ ID NO:53, SEQ ID NO:61, SEQ ID NO:62, or SEQ ID NO:63.

Embodiment 324. The method of embodiment 99, wherein the target-hybridizing sequence of (a) is SEQ ID NO:3, SEQ ID NO:48, SEQ ID NO:49, SEQ ID NO:54, SEQ ID NO:55, or SEQ ID NO:56.

Embodiment 325. The method of embodiment 103 or 324, wherein the target-hybridizing sequence of (b)(i) is SEQ ID NO:50, SEQ ID NO:51, SEQ ID NO:52, SEQ ID NO:9, SEQ ID NO:57, SEQ ID NO:58, SEQ ID NO:59, or SEQ ID NO:60.

Embodiment 326. The method of embodiment 99, wherein the target-hybridizing sequence of (a) is SEQ ID NO:3, SEQ ID NO:48, or SEQ ID NO:49.

Embodiment 327. The method of embodiment 103 or 326, wherein the target-hybridizing sequence of (b)(i) is SEQ ID NO:50, SEQ ID NO:51, SEQ ID NO 52, or SEQ ID NO:9.

Embodiment 328. The method of embodiment 99, wherein the target-hybridizing sequence of (a) is SEQ ID NO:54, SEQ ID NO:55, or SEQ ID NO:56.

Embodiment 329. The method of embodiment 103 or 328, wherein the target-hybridizing sequence of (b)(i) is SEQ ID NO:57, SEQ ID NO:58, SEQ ID NO:59, or SEQ ID NO:60.

Embodiment 330. The method of embodiment 101, wherein
the first amplification oligomer as in (a) comprises the target-hybridizing sequence shown in SEQ ID NO:3,
the second amplification oligomer as in (a) comprises the target-hybridizing sequence shown in SEQ ID NO:48, and
the third amplification oligomer as in (a) comprises the target-hybridizing sequence shown in SEQ ID NO:49.

Embodiment 331. The method of embodiment 105, wherein
the first amplification oligomer as in (b)(i) comprises the target-hybridizing sequence shown in SEQ ID NO:50,
the second amplification oligomer as in (b)(i) comprises the target-hybridizing sequence shown in SEQ ID NO:51,
the third amplification oligomer as in (b)(i) comprises the target-hybridizing sequence shown in SEQ ID NO:52, and
the fourth amplification oligomer as in (b)(i) comprises the target-hybridizing sequence shown in SEQ ID NO:9.

Embodiment 332. The method of embodiment 101, wherein
the first amplification oligomer as in (a) comprises the target-hybridizing sequence shown in SEQ ID NO:54,
the second amplification oligomer as in (a) comprises the target-hybridizing sequence shown in SEQ ID NO:55, and
the third amplification oligomer as in (a) comprises the target-hybridizing sequence shown in SEQ ID NO:56.

Embodiment 333. The method of embodiment 105, wherein
the first amplification oligomer as in (b)(i) comprises the target-hybridizing sequence shown in SEQ ID NO:57,
the second amplification oligomer as in (b)(i) comprises the target-hybridizing sequence shown in SEQ ID NO:58,
the third amplification oligomer as in (b)(i) comprises the target-hybridizing sequence shown in SEQ ID NO:59, and
the fourth amplification oligomer as in (b)(i) comprises the target-hybridizing sequence shown in SEQ ID NO:60.

Embodiment 334. The method of embodiment 111, wherein the at least one detection probe oligomer target-hybridizing sequence is SEQ ID NO:53, SEQ ID NO:16, or SEQ ID NO:17.

Embodiment 335. The method of embodiment 111, wherein the at least one detection probe oligomer target-hybridizing sequence is SEQ ID NO:61, SEQ ID NO:62, or SEQ ID NO:63.

Embodiment 336. The method of embodiment 116, wherein
the first detection probe oligomer comprises the target-hybridizing sequence shown in SEQ ID NO:53,
the second detection probe oligomer comprises the target-hybridizing sequence shown in SEQ ID NO: 16, and
the third detection probe oligomer comprises the target-hybridizing sequence shown in SEQ ID NO:17.

Embodiment 337. The method of embodiment 116, wherein
the first detection probe oligomer comprises the target-hybridizing sequence shown in SEQ ID NO:61,
the second detection probe oligomer comprises the target-hybridizing sequence shown in SEQ ID NO:62, and
the third detection probe oligomer comprises the target-hybridizing sequence shown in SEQ ID NO:63.

Embodiment 338. The method of embodiment 122, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:57, SEQ ID NO:58, SEQ ID NO:59, or SEQ ID NO:60.

Embodiment 339. The method of embodiment 123, wherein the at least one detection probe oligomer target-hybridizing sequence is SEQ ID NO:53, SEQ ID NO:61, SEQ ID NO:62, or SEQ ID NO:63.

Embodiment 340. The method of embodiment 124, wherein at least one detection probe oligomer target-hybridizing sequence is SEQ ID NO:53, SEQ ID NO:61, SEQ ID NO:62, or SEQ ID NO:63.

Embodiment 341. The oligonucleotide of embodiment 137, wherein the HAdV-specific target-hybridizing sequence is SEQ ID NO:3, SEQ ID NO:48, SEQ ID NO:49, SEQ ID NO:50, SEQ ID NO:51, SEQ ID NO:52, SEQ ID NO:9, SEQ ID NO:53, SEQ ID NO: 16, SEQ ID NO:17, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, or SEQ ID NO:63.

Embodiment 342. The composition or kit of embodiment 146 or 147, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 343. The formulation of embodiment 148 or 149, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 344. The method of embodiment 150 or 151, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 345. The composition or kit of embodiment 154, wherein the target-hybridizing sequence of (a)(ii) is SEQ ID NO:64 or SEQ ID NO:28.

Embodiment 346. The composition or kit of embodiment 155, wherein
the first amplification oligomer as in (a)(ii) comprises the target-hybridizing shown in SEQ ID NO:64, and/or
the second amplification oligomer as in (a)(ii) comprises the target-hybridizing sequence shown in SEQ ID NO:28.

Embodiment 347. The composition or kit of embodiment 160, wherein the target-hybridizing sequence of (c)(ii) is SEQ ID NO:34 or SEQ ID NO:67.

Embodiment 348. The composition or kit of embodiment 161, wherein
the first amplification oligomer as in (c)(ii) comprises the target-hybridizing shown in SEQ ID NO:34, and/or
the second amplification oligomer as in (c)(ii) comprises the target-hybridizing sequence shown in SEQ ID NO:67.

Embodiment 349. The composition or kit of embodiment 166, wherein the target-hybridizing sequence of (f)(i) is SEQ ID NO:70 or SEQ ID NO:71.

Embodiment 350. The composition or kit of embodiment 167, wherein
the first amplification oligomer as in (f)(i) comprises the target-hybridizing shown in SEQ ID NO:70, and/or
the second amplification oligomer as in (f)(i) comprises the target-hybridizing sequence shown in SEQ ID NO:71.

Embodiment 351. The composition or kit of embodiment 168, wherein the target-hybridizing sequence of (f)(ii) is SEQ ID NO:72 or SEQ ID NO:73.

Embodiment 352. The composition or kit of embodiment 169, wherein
the first amplification oligomer as in (f)(ii) comprises the target-hybridizing shown in SEQ ID NO: 72, and/or
the second amplification oligomer as in (f)(ii) comprises the target-hybridizing sequence shown in SEQ ID NO:73.

Embodiment 353. The composition or kit of embodiment 172, wherein the at least one detection probe target-hybridizing sequence is SEQ ID NO:65 or SEQ ID NO:66.

Embodiment 354. The composition or kit of embodiment 173, wherein (i) the first detection probe target-hybridizing sequence is SEQ ID NO:65, and/or (ii) the second detection probe target-hybridizing sequence is SEQ ID NO:66.

Embodiment 355. The composition or kit of embodiment 174, wherein the at least one detection probe target-hybridizing sequence is SEQ ID NO:68.

Embodiment 356. The composition or kit of embodiment 175, wherein the at least one detection probe target-hybridizing sequence is SEQ ID NO:69.

Embodiment 357. The composition or kit of embodiment 176, wherein the at least one detection probe target-hybridizing sequence is SEQ ID NO:74.

Embodiment 358. The composition or kit of embodiment 180, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:64, SEQ ID NO:67, SEQ ID NO: 70, SEQ ID NO: 71, SEQ ID NO: 72, or SEQ ID NO: 73.

Embodiment 359. The composition or kit of embodiment 181, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 360. The composition or kit of embodiment 182, wherein at least one amplification oligomer target-hybridizing sequence is SEQ ID NO:55, SEQ ID NO:56, SEQ ID NO:57, SEQ ID NO:58, SEQ ID NO:59, SEQ ID NO: 60, SEQ ID NO:64, SEQ ID NO: 70, or SEQ ID NO:73.

Embodiment 361. The composition or kit of embodiment 183 or 190, wherein at least one detection probe target-hybridizing sequence is SEQ ID NO:53, SEQ ID NO:61, SEQ ID NO:62, SEQ ID NO:63, SEQ ID NO:65, SEQ ID NO:66, SEQ ID NO:68, SEQ ID NO:69, or SEQ ID NO:46.

Embodiment 362. The formulation of embodiment 193, wherein the target-hybridizing sequence of (a)(ii) is SEQ ID NO:64 or SEQ ID NO:28.

Embodiment 363. The formulation of embodiment 194, wherein
the first amplification oligomer as in (a)(ii) comprises the target-hybridizing shown in SEQ ID NO:64, and/or
the second amplification oligomer as in (a)(ii) comprises the target-hybridizing sequence shown in SEQ ID NO:28.

Embodiment 364. The formulation of embodiment 199, wherein the target-hybridizing sequence of (c)(ii) is SEQ ID NO:34 or SEQ ID NO:67.

Embodiment 365. The formulation of embodiment 200, wherein
the first amplification oligomer as in (c)(ii) comprises the target-hybridizing shown in SEQ ID NO:34, and/or
the second amplification oligomer as in (c)(ii) comprises the target-hybridizing sequence shown in SEQ ID NO:67.

Embodiment 366. The formulation of embodiment 205, wherein the target-hybridizing sequence of (f)(i) is SEQ ID NO:70 or SEQ ID NO:71.

Embodiment 367. The formulation of embodiment 206, wherein
the first amplification oligomer as in (f)(i) comprises the target-hybridizing shown in SEQ ID NO: 70, and/or
the second amplification oligomer as in (f)(i) comprises the target-hybridizing sequence shown in SEQ ID NO:71.

Embodiment 368. The formulation of embodiment 207, wherein the target-hybridizing sequence of (f)(ii) is SEQ ID NO:72 or SEQ ID NO:73.

Embodiment 369. The formulation of embodiment 208, wherein
the first amplification oligomer as in (f)(ii) comprises the target-hybridizing shown in SEQ ID NO:72, and/or
the second amplification oligomer as in (f)(ii) comprises the target-hybridizing sequence shown in SEQ ID NO:73.

Embodiment 370. The formulation of embodiment 211, wherein the at least one detection probe target-hybridizing sequence is SEQ ID NO:65 or SEQ ID NO:66.

Embodiment 371. The formulation of embodiment 212, wherein (i) the first detection probe target-hybridizing sequence is SEQ ID NO:65, and/or (ii) the second detection probe target-hybridizing sequence is SEQ ID NO:66.

Embodiment 372. The formulation of embodiment 213, wherein the at least one detection probe target-hybridizing sequence is SEQ ID NO:68.

Embodiment 373. The formulation of embodiment 214, wherein the at least one detection probe target-hybridizing sequence is SEQ ID NO:69.

Embodiment 374. The formulation of embodiment 215, wherein the at least one detection probe target-hybridizing sequence is SEQ ID NO:74.

Embodiment 375. The formulation of embodiment 219, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:64, SEQ ID NO:67, SEQ ID NO: 70, SEQ ID NO:71, SEQ ID NO: 72, or SEQ ID NO: 73.

Embodiment 376. The formulation of embodiment 220, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 377. The formulation of embodiment 221, wherein at least one amplification oligomer target-hybridizing sequence is SEQ ID NO:55, SEQ ID NO:56, SEQ ID NO:57, SEQ ID NO:58, SEQ ID NO:59, SEQ ID NO:60, SEQ ID NO:64, SEQ ID NO: 70, or SEQ ID NO:73.

Embodiment 378. The formulation of embodiment 222 or 231, wherein at least one detection probe target-hybridizing sequence is SEQ ID NO:53, SEQ ID NO:61, SEQ ID NO:62, SEQ ID NO:63, SEQ ID NO:65, SEQ ID NO:66, SEQ ID NO:68, SEQ ID NO:69, or SEQ ID NO:46.

Embodiment 379. The method of embodiment 245, wherein the target-hybridizing sequence of (a)(ii) is SEQ ID NO:64 or SEQ ID NO:28.

Embodiment 380. The method of embodiment 246, wherein
the first amplification oligomer as in (a)(ii) comprises the target-hybridizing shown in SEQ ID NO:64, and/or
the second amplification oligomer as in (a)(ii) comprises the target-hybridizing sequence shown in SEQ ID NO:28.

Embodiment 381. The method of embodiment 251, wherein the target-hybridizing sequence of (c)(ii) is SEQ ID NO:34 or SEQ ID NO:67.

Embodiment 382. The method of embodiment 252, wherein
the first amplification oligomer as in (c)(ii) comprises the target-hybridizing shown in SEQ ID NO:34, and/or
the second amplification oligomer as in (c)(ii) comprises the target-hybridizing sequence shown in SEQ ID NO:67.

Embodiment 383. The method of embodiment 257, wherein the target-hybridizing sequence of (f)(i) is SEQ ID NO:70 or SEQ ID NO:71.

Embodiment 384. The method of embodiment 258, wherein
the first amplification oligomer as in (f)(i) comprises the target-hybridizing shown in SEQ ID NO: 70, and/or
the second amplification oligomer as in (f)(i) comprises the target-hybridizing sequence shown in SEQ ID NO:71.

Embodiment 385. The method of embodiment 259, wherein the target-hybridizing sequence of (f)(ii) is SEQ ID NO:72 or SEQ ID NO:73.

Embodiment 386. The method of embodiment 260, wherein
the first amplification oligomer as in (f)(ii) comprises the target-hybridizing shown in SEQ ID NO:72, and/or
the second amplification oligomer as in (f)(ii) comprises the target-hybridizing sequence shown in SEQ ID NO:73.

Embodiment 387. The method of embodiment 263, wherein the at least one detection probe target-hybridizing sequence is SEQ ID NO:65 or SEQ ID NO:66.

Embodiment 388. The method of embodiment 264, wherein (i) the first detection probe target-hybridizing sequence is SEQ ID NO:65, and/or (ii) the second detection probe target-hybridizing sequence is SEQ ID NO:66.

Embodiment 389. The method of embodiment 265, wherein the at least one detection probe target-hybridizing sequence is SEQ ID NO:68.

Embodiment 390. The method of embodiment 266, wherein the at least one detection probe target-hybridizing sequence is SEQ ID NO:69.

Embodiment 391. The method of embodiment 267, wherein the at least one detection probe target-hybridizing sequence is SEQ ID NO:74.

Embodiment 392. The method of embodiment 272, wherein at least one amplification oligomer target-hybridizing sequence is SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:64, SEQ ID NO:67, SEQ ID NO:70, SEQ ID NO:71, SEQ ID NO: 72, or SEQ ID NO: 73.

Embodiment 393. The method of embodiment 273, wherein at least one amplification oligomer target-hybridizing sequence is SEQ ID NO:55, SEQ ID NO:56, SEQ ID NO:57, SEQ ID NO:58, SEQ ID NO:59, or SEQ ID NO:60.

Embodiment 394. The method of embodiment 274, wherein at least one amplification oligomer target-hybridizing sequence is SEQ ID NO:55, SEQ ID NO:56, SEQ ID NO:57, SEQ ID NO:58, SEQ ID NO:59, SEQ ID NO:60, SEQ ID NO:64, SEQ ID NO: 70, or SEQ ID NO:73.

Embodiment 395. The method of embodiment 275, wherein at least one detection probe target-hybridizing sequence is SEQ ID NO:53, SEQ ID NO:61, SEQ ID NO:62, SEQ ID NO:63, SEQ ID NO:65, SEQ ID NO:66, SEQ ID NO: 68, SEQ ID NO:69, or SEQ ID NO:46.

Embodiment 396. The oligonucleotide of embodiment 287, wherein the HAdV-specific target-hybridizing sequence is SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO: 60, SEQ ID NO:61, SEQ ID NO:62, SEQ ID NO:63, SEQ ID NO:64, SEQ ID NO:65, SEQ ID NO: 66, SEQ ID NO:67, SEQ ID NO:68, SEQ ID NO:69, SEQ ID NO: 70, SEQ ID NO: 71, SEQ ID NO: 72, SEQ ID NO: 73, or SEQ ID NO 74.

These and other aspects and embodiments will become evident upon reference to the following detailed description and the attached drawings.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

When a value is expressed as “about” X or “approximately” X, the stated value of X will be understood to be accurate to +10%. When the term “about” is used in reference to a nucleotide position of an adenovirus target nucleic acid (e.g., “about nucleotide position 14714 of SEQ ID NO:25”), the term will be understood to mean any nucleotide position within 10 nucleotides upstream or downstream of the stated position.

All ranges are to be interpreted as encompassing the endpoints in the absence of express exclusions such as “not including the endpoints”; thus, for example, “from 0 to 9” includes the values 0 and 9.

“Sample” includes any specimen that may contain a human adenovirus, including components thereof, such as nucleic acids or fragments of nucleic acids. Samples include “biological samples” which include any tissue or material derived from a living or dead human. The biological sample may be treated to physically or mechanically disrupt tissue or cell structure, thus releasing intracellular components into a solution which may further contain enzymes, buffers, salts, detergents, and the like, which are used to prepare a biological sample for analysis. Also, samples may include processed samples such as samples in which one or more components have been concentrated or purified. Processed samples include, e.g., those obtained from passing samples over or through a filtering device, or following centrifugation, or by adherence to a medium, matrix, or support. In addition, a sample may be an individual sample (i.e., a sample derived from a single subject) or a pooled sample (i.e., a sample prepared by pooling a plurality of individual samples).

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Nucleic acid” and “polynucleotide” refer to a multimeric compound comprising nucleotides and/or nucleotide analogs linked together to form a biopolymer. The biopolymers include conventional RNA, conventional DNA, mixed RNA-DNA, and nucleotide analog containing versions thereof. A nucleic acid “backbone” may be made up of a variety of linkages, including one or more of sugar-phosphodiester linkages, peptide-nucleic acid bonds (“peptide nucleic acids” or PNA), phosphorothioate linkages, methylphosphonate linkages, or combinations thereof. Sugar moieties of a nucleic acid may be ribose, deoxyribose, or similar compounds with substitutions, e.g., analogs with a methoxy, fluoro or halide group at the 2′ position of the ribose (also referred to herein as “2′-O-Me” or “2′-methoxy” or 2′-fluoro, or “2′-halide”). Nitrogenous bases may be conventional bases, adenine (A), uracil (U), guanine (G), thymine (T), and cytosine (C), and analogs thereof (e.g., inosine. 5 methyl 2′ deoxycytidine (“5-Me-dC,” “5MeC,” or “5-methyl cytosine”), isoguanine, propyne dC (pdC), or propyne dU (pdU)). As used in the present disclosure, pdC is considered a cytosine analogue and is also denoted herein as “C^{dC}”; and pdU is considered a thymine analogue and is also denoted herein as “T^{dU}” Nucleic acids may include one or more “abasic” residues where the backbone includes no nitrogenous base for position(s) of the polymer.

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. Unless the context clearly dictates otherwise, reference to a nucleic acid of coronavirus includes the RNA and DNA equivalents and DNA/RNA chimerics thereof.

The phrase “and including from 0 to 9 nucleotide analogs,” as used herein following a list of alternative nucleotide sequences, means that the referenced sequences include equivalents of each sequence having from 0 to 9 nucleotide analogs. Thus, when used immediately after the phrase “or an RNA equivalent or a DNA/RNA chimeric thereof” following one or more SEQ ID NOs., the phrase “and including from 0 to 9 nucleotide analogs” refers to each SEQ ID NO as well as to RNA equivalents and DNA/RNA chimerics of each SEQ ID NO.

“Oligomer,” “oligonucleotide,” or “oligo” refers to a nucleic acid of generally less than 1,000 nucleotides (nt), including those in a size range having a lower limit of about 5 nt and an upper limit of about 900 nt. The term oligonucleotide does not denote any particular function to the reagent; rather, it is used generically to cover all such reagents described herein. Oligomers may be referred to by a functional name (e.g., capture probe, primer, or promoter primer) but those skilled in the art will understand that such terms refer to oligomers.

A “target nucleic acid” as used herein is a nucleic acid comprising a target sequence to be amplified. Target nucleic acids may be DNA or RNA and may be either single-stranded or double-stranded. The target nucleic acid may include other sequences besides the target sequence, which may not be amplified.

The term “target sequence” or “target nucleic acid sequence” as used herein refers to the particular nucleotide sequence of the target nucleic acid that is to be amplified and/or detected. The “target sequence” includes the complexing sequences to which oligonucleotides (e.g., priming oligonucleotides and/or promoter oligonucleotides) complex during an amplification processes (e.g., PCR, TMA). Unless the context clearly dictates otherwise, where the target nucleic acid is originally single-stranded, the term “target sequence” will also refer to the sequence complementary to the “target sequence” as present in the target nucleic acid, and where the target nucleic acid is originally double-stranded, the term “target sequence” refers to both the sense (+) and antisense (−) strands.

“Target-hybridizing sequence” or “target-specific sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence.

“Non-target-specific sequence” or “non-target-hybridizing sequence” as used herein refers to a region of an oligomer sequence, wherein said region does not stably hybridize with a target sequence under standard hybridization conditions. Oligomers with non-target-specific sequences include, but are not limited to, promoter primers, promoter providers, target capture oligomers, torches, and molecular beacons.

The term “target a sequence,” as used herein in reference to a region of a human adenovirus nucleic acid, refers to a process whereby an oligonucleotide hybridizes to a target sequence in a manner that allows for amplification and detection as described herein. In one embodiment, the oligonucleotide is complementary with the targeted human adenovirus nucleic acid sequence and contains no mismatches. In another embodiment, the oligonucleotide is complementary but contains 1, 2, 3, 4, or 5 mismatches with the targeted human adenovirus nucleic acid sequence.

The term “configured to” denotes an actual arrangement of the polynucleotide sequence configuration of a referenced oligonucleotide target-hybridizing sequence. For example, amplification oligomers that are configured to generate a specified amplicon from a target sequence have polynucleotide sequences that hybridize to the target sequence and can be used in an amplification reaction to generate the amplicon. Also, as an example, oligonucleotides that are configured to specifically hybridize to a target sequence have a polynucleotide sequence that specifically hybridizes to the referenced sequence under stringent hybridization conditions.

The term “configured to specifically hybridize to” as used herein means that the target-hybridizing region of an amplification oligonucleotide, detection probe, or other oligonucleotide is designed to have a polynucleotide sequence that could target a sequence of the referenced human adenovirus target sequence. The oligonucleotide is designed to function as a component of an assay for amplification and detection of human adenovirus target nucleic acid from a sample, and therefore is designed to target human adenovirus in the presence of other nucleic acids commonly found in testing samples. “Specifically hybridize to” does not mean exclusively hybridize to, as some small level of hybridization to non-target nucleic acids may occur, as is understood in the art. Rather. “specifically hybridize to” means that the oligonucleotide is configured to function in an assay to primarily hybridize the target so that an accurate detection of target nucleic acid in a sample can be determined.

An “amplification oligonucleotide” or “amplification oligomer” is an oligonucleotide that hybridizes to a target nucleic acid and participates in a nucleic acid amplification reaction, e.g., serving as a primer. Amplification oligomers can have 3′ ends that are extended by polymerization as part of the nucleic acid amplification reaction. Amplification oligomers can alternatively have 3′ ends that are not extended by polymerization, but provide a component that facilitates nucleic acid amplification, e.g., a promoter sequence joined 5′ to the target hybridizing sequence of the amplification oligomer. Such an amplification oligomer is referred to as a promoter provider. Amplification oligomers that provide both a 3′ target hybridizing region that is extendable by polymerization and a 5′ promoter sequence are referred to as promoter primers. Amplification oligomers may be optionally modified to include 5′ non-target hybridizing regions such as tags, promoters (as mentioned), or other sequences used or useful for manipulating or amplifying the primer or target oligonucleotide.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), and polymerase chain reaction (PCR) (e.g., U.S. Pat. Nos. 4,683,195, 4,683,202, and 4,800,159).

By “amplicon” or “amplification product” is meant a nucleic acid molecule generated in a nucleic acid amplification reaction and which is derived from a target nucleic acid. An amplicon or amplification product contains a target nucleic acid sequence that may be of the same or opposite sense as the target nucleic acid.

As used herein, the term “relative fluorescence unit” (“RFU”) is a unit of measurement of fluorescence intensity. RFU varies with the characteristics of the detection means used for the measurement and can be used as a measurement to compare relative intensities between samples and controls.

“Detection probe oligomer,” “detection probe,” or “probe” refers to an oligomer that hybridizes specifically to a target sequence, including an amplified product, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification.

By “stringent hybridization conditions.” or “stringent conditions” is meant conditions permitting an oligomer to preferentially hybridize to a target sequence and not to nucleic acid derived from a closely related non-target nucleic acid (i.e., conditions permitting an oligomer to hybridize to its target sequence to form a stable oligomer:target hybrid, but not form a sufficient number of stable oligomer:non-target hybrids, so as to allow for amplification and/or detection of target nucleic acids but not non-targeted organisms). While the definition of stringent hybridization conditions does not vary, the actual reaction environment that can be used for stringent hybridization may vary depending upon factors including the GC content and length of the oligomer, the degree of similarity between the oligomer sequence and sequences of non-target nucleic acids that may be present in the test sample, and the target sequence. Hybridization conditions include the temperature and the composition of the hybridization reagents or solutions. Stringent hybridization conditions are readily ascertained by those having ordinary skill in the art.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal. Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridinium ester (“AE”) compound), and fluorescent compound (i.e., fluorophore). Fluorophores may be used in combination with a quencher molecule that absorbs light when in close proximity to the fluorophore to diminish background fluorescence. Detectably labeled probes include, e.g., TaqMan™ probes. AE-labeled probes, molecular torches, and molecular beacons.

A “non-extendable” oligomer includes a blocking moiety at or near its 3′-terminus to prevent extension. A blocking group near the 3′ end is in some embodiments within five residues of the 3′ end and is sufficiently large to limit binding of a polymerase to the oligomer. In other embodiments, a blocking group is covalently attached to the 3′ terminus. Suitable blocking groups include, e.g., alkyl groups, non-nucleotide linkers, alkane-diol dideoxynucleotide residues, cordycepin, 3-deoxy nucleotides, 3-phosphorylated nucleotides, inverted nucleotides, proteins, peptides, and labels such as fluorophores or quenchers.

References, particularly in the embodiments, to “the sequence of SEQ ID NO:X” refer to the sequence of nucleotides and/or nucleotide analogs linked together to form a biopolymer. Reference to a sequence by SEQ ID NO: does not connote the identity of the backbone (e.g., RNA, 2′-O-Me RNA, or DNA) or any nucleobase modifications (e.g., methylation of cytosine residues (“5MeC”)) unless the context clearly dictates otherwise. In some instances, the sequence of a SEQ ID NO: is followed by the statement “including from [x-y] nucleotide analogs”; it is understood that the nucleotide analogs may be substitutions within the sequence of the SEQ ID NO:. Unless the context clearly dictates otherwise, reference to a sequence by SEQ ID NO: includes reference to its complementary sequence (e.g., reference to the sequence 5′-ttagc-3′ includes reference to the sequence 5′-gctaa-3′).

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

The term “specificity.” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

### DETAILED DESCRIPTION

Provided herein are compositions, kits, formulations, and methods for amplifying and/or detecting human adenovirus nucleic acid in a sample. Preferably, the samples are biological samples. The compositions, kits, and methods provide oligonucleotide sequences that target the 3′ region of the penton gene sequence of human adenovirus or their complementary sequences. Such oligonucleotides may be used as amplification oligonucleotides, which may include primers, promoter primers, blocked oligonucleotides, and promoter provider oligonucleotides, whose functions have been described previously (see, e.g., U.S. Pat. Nos. 4,683,195; 4,683,202; 4,800,159; 5,399,491; 5,554,516; 5,824,518; and 7,374,885; each incorporated by reference herein). Other oligonucleotides may be used as probes for detecting amplified sequences of human adenovirus, or for capture of human adenovirus target nucleic acid.

The methods provide for the sensitive and specific detection of human adenovirus nucleic acids. The methods include performing a nucleic acid amplification of a human adenovirus target region, and detecting the amplified product by, for example, specifically hybridizing the amplified product with a nucleic acid detection probe that provides a signal to indicate the presence of human adenovirus in the sample. The amplification step includes contacting the sample with one or more amplification oligomers specific for a target sequence in a human adenovirus target nucleic acid to produce an amplified product if human adenovirus nucleic acid is present in the sample. Amplification synthesizes additional copies of the target sequence or its complement by using at least one nucleic acid polymerase and an amplification oligomer to produce the copies from a template strand (e.g., by extending the sequence from a primer using the template strand). One embodiment for detecting the amplified product uses a hybridizing step that includes contacting the amplified product with at least one detection probe oligomer specific for a sequence amplified by the selected amplification oligomers, e.g., a sequence contained in the target sequence flanked by a pair of selected amplification oligomers.

In some aspects, the compositions are configured to specifically hybridize to a penton gene target sequence from each human adenovirus subtype. In some aspects, the compositions are configured to specifically hybridize to a penton gene target sequence from each of human adenovirus subtypes A-F. In other aspects, the compositions are configured to specifically hybridize to a penton gene target sequence from at least two of human adenovirus subtypes A-F.

In some aspects, the compositions are configured to specifically hybridize to human adenovirus (HAdV) nucleic acid with minimal cross-reactivity to one or more non-HAdV pathogens. In some aspects, the compositions are configured to specifically hybridize to human adenovirus nucleic acid with minimal cross-reactivity to one or more non-HAdV pathogens listed in Table 23 (see Example 11, infra). For example, in some embodiments, the compositions are configured to specifically hybridize to human adenovirus nucleic acid with minimal cross-reactivity to one or more pathogens selected from varicella zoster virus, Epstein-Barr virus, BK virus, cytomegalovirus, Candida albicans, Chlamydia trachomatis, human immunodeficiency virus type 1 (HIV-1), human immunodeficiency virus type 2 (HIV-2), Dengue virus type 1, Dengue virus type 2, Dengue virus type 3, Dengue virus type 4, herpes simplex virus type 1 (HSV-1), herpes simplex virus type 2 (HSV-2), human papillomavirus type 18 (HPV18), human papillomavirus type 16 (HPV16), human herpes virus type 6A (HHV-6A), human herpes virus type 6B (HHV-6B), human herpes virus type 7 (HHV-7), human herpes virus type 8 (HHV-8), human T-lymphotropic virus type I (HTLV-I), human T-lymphotropic virus type II (HTLV-II), hepatitis A virus (HAV), hepatitis B virus (HBV), hepatitis C virus (HCV), Mycobacterium smegmatis, Neisseria gonorrhoeae, Propionibacterium acnes, West Nile virus, vaccinia virus, Trichomonas vaginalis, Staphylococcus epidermidis, human parvovirus, Staphylococcus aureus, Mycobacterium gordonae. In one aspect, the compositions are part of a multiplex system that further includes components and methods for detecting one of more pathogens that are not human adenovirus (e.g., one or more non-HAdV pathogens such as, for example, one or more of the pathogens listed in Table 23).

In some aspects, there are provided methods for utilizing an oligomer or oligomer combination as described herein. Any method disclosed herein is also to be understood as a disclosure of corresponding uses of materials involved in the method directed to the purpose of the method. Any of the oligomers comprising a human adenovirus target-hybridizing sequence and any combinations (e.g., kits and compositions) comprising such an oligomer are to be understood as also disclosed for use in detecting or quantifying human adenovirus and for use in the preparation of a composition for detecting or quantifying human adenovirus.

Broadly speaking, methods may comprise one or more of the following components: target capture, in which human adenovirus nucleic acid (e.g., from a sample, such as a clinical sample) is annealed to a capture oligomer; isolation, e.g., washing, to remove material not associated with a capture oligomer; amplification; and amplicon detection, e.g., amplicon quantification, which may be performed in real time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two components listed adjacently above, e.g., washing and amplification, or amplification and detection.

Amplifying a human adenovirus target sequence utilizes an in vitro amplification reaction using at least two amplification oligomers that flank a target region to be amplified. Particularly suitable oligomer combinations for amplification of human adenovirus target regions are described herein. In some embodiments, an oligomer combination includes at least two amplification oligomers capable of amplifying a target region of human adenovirus target nucleic acid corresponding to a region of SEQ ID NO:25 from about nucleotide position 14714 to about nucleotide position 14841 (e.g., at least two amplification oligomers capable of generating a human adenovirus amplicon comprising or consisting of a sequence as shown in SEQ ID NO:22 or SEQ ID NO:23); in some such embodiments. (a) at least one amplification oligomer comprises a target-hybridizing sequence as shown in SEQ ID NO: 18, and (b) at least one amplification oligomer comprises a target-hybridizing sequence as shown in SEQ ID NO:21. In other embodiments, an oligomer combination includes at least two amplification oligomer capable of amplifying a target region of human adenovirus target nucleic acid corresponding to a region of SEQ ID NO:25 from about nucleotide position 14714 to about nucleotide position 14795 (e.g., at least two amplification oligomers capable of generating a human adenovirus amplicon comprising or consisting of a sequence as shown in SEQ ID NO:24); in some such embodiments. (a) at least one amplification oligomer comprises a target-hybridizing sequence as shown in SEQ ID NO: 18, and (b) at least one amplification oligomer comprises a target-hybridizing sequence as shown in SEQ ID NO:20. Exemplary amplification oligomers for amplifying the human adenovirus target region are listed in Tables 36, 26, and 27 infra, and particular combinations of first and second amplification oligomers for each of the first and second target regions are set forth herein (see, e.g., Embodiments section, supra, and Examples 1, 3 and 6, infra (including Tables 1 and 13).

In certain embodiments, the method further includes purifying the human adenovirus target nucleic acid from other components in the sample, e.g., before an amplification, such as before a capture step. Such purification may include methods of separating and/or concentrating organisms contained in a sample from other sample components, or removing or degrading non-nucleic acid sample components, e.g., protein, carbohydrate, salt, lipid, etc. In particular embodiments, a target nucleic acid is captured specifically or non-specifically and separated from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains human adenovirus nucleic acid and other sample components.

Target capture typically occurs in a solution phase mixture that contains one or more capture probe oligomers that hybridize to the human adenovirus (HAdV) target sequence under hybridizing conditions. For embodiments comprising a capture probe tail, the HAdV-target:capture-probe complex is captured by adjusting the hybridization conditions so that the capture probe tail hybridizes to an immobilized probe. Certain embodiments use a particulate solid support, such as paramagnetic beads. Selective and non-specific target capture methods are also described, e.g., in U.S. Pat. No. 6,110,678 and International Patent Application Pub. Nos. WO 2008/016988 & WO 2021/097358, each incorporated by reference herein.

Isolation can follow capture, where, for example, the complex on the solid support is separated from other sample components. Isolation can be accomplished by any appropriate technique, e.g., washing a support associated with the human adenovirus target-sequence one or more times (e.g., two or three times) to remove other sample components and/or unbound oligomer. In embodiments using a particulate solid support, such as paramagnetic beads, particles associated with the human adenovirus target may be suspended in a washing solution and retrieved from the washing solution, in some embodiments by using magnetic attraction. To limit the number of handling steps, the human adenovirus target nucleic acid may be amplified by simply mixing the target sequence in the complex on the support with amplification oligomers and proceeding with amplification steps.

Sample preparation may also include pooling a plurality of samples into a single pooled batch. Preferably for pooling, an aliquot of each sample is pooled into the larger batch. The larger batch of pooled samples can be from a plurality of samples wherein the plurality is from 2 to about 200 individual samples.

Exponentially amplifying a human adenovirus target sequence utilizes an in vitro amplification reaction using at least two amplification oligomers that flank a target region to be amplified. The amplification reaction can be cycled or isothermal.

A detection step may be performed using any of a variety of known techniques to detect a signal specifically associated with the amplified target sequence, such as, e.g., by hybridizing the amplification product with a labeled detection probe and detecting a signal resulting from the labeled probe (including from label released from the probe following hybridization in some embodiments). In some embodiments, the labeled probe comprises a second moiety, such as a quencher or other moiety that interacts with the first label, as discussed above. The detection step may also provide additional information on the amplified sequence, such as, e.g., all or a portion of its nucleic acid base sequence. Detection may be performed after the amplification reaction is completed or may be performed simultaneously with amplifying the target region, e.g., in real time. In embodiments that detect the amplified product near or at the end of the amplification step, a linear detection probe may be used to provide a signal to indicate hybridization of the probe to the amplified product. One example of such detection uses a luminescently labeled probe that hybridizes to target nucleic acid. The luminescent label is then hydrolyzed from non-hybridized probe. Detection is performed by chemiluminescence using a luminometer. (See, e.g., International Patent Application Pub. No. WO 89/002476, incorporated by reference herein). In other embodiments that use real-time detection, the detection probe may be a hairpin probe such as, for example, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In other embodiments for real-time detection, the detection probe is a linear oligomer such as, e.g., an oligomer labeled with both a fluorophore and a quenching moiety (e.g., a TaqMan probe). Such probes may comprise target-hybridizing sequences and non-target-hybridizing sequences. Various forms of such probes have been described previously (see, e.g., U.S. Pat. Nos. 5,210,015; 5,487,972; 5,118,801; 5,312,728; 5,925,517; 6,150,097; 6,849,412; 6,835,542; 6,534,274; and 6,361,945; and US Patent Application Pub. Nos. 20060068417A1 and 20060194240A1; each incorporated by reference herein). Exemplary HAdV-specific detection probe oligomers are listed in Table 36, 26, and 27, infra, and are further set forth in the Embodiments section, supra, and Examples, infra (including, e.g., their use in combination with at least two HAdV-specific amplification oligomers for detection of human adenovirus target nucleic acid).

Assays for detection of the human adenovirus nucleic acid may optionally include a non-HAdV internal control (IC) nucleic acid that is amplified and detected in the same assay reaction mixtures by using amplification and detection oligomers specific for the IC sequence. IC nucleic acid sequences can be, e.g., a DNA plasmid, an RNA template sequence (e.g., an in vitro transcript), or a synthetic nucleic acid that is spiked into a sample. Alternatively, the IC nucleic acid sequence may be a cellular component, which may be from exogenous cellular sources or endogenous cellular sources relative to the specimen. In these instances, an internal control nucleic acid is co-amplified with the human adenovirus nucleic acid in the amplification reaction mixtures. The internal control amplification product and the human adenovirus target sequence amplification product can be detected independently.

In certain embodiments, amplification and detection of a signal from an amplified IC sequence demonstrates that the assay reagents, conditions, and performance of assay steps were properly used in the assay if no signal is obtained for the intended target human adenovirus nucleic acid (e.g., samples that test negative for human adenovirus). An IC may also be used as an internal calibrator for the assay when a quantitative result is desired, i.e., the signal obtained from the IC amplification and detection is used to set a parameter used in an algorithm for quantitating the amount of human adenovirus nucleic acid in a sample based on the signal obtained for an amplified human adenovirus target sequence. ICs are also useful for monitoring the integrity of one or more steps in an assay. The primers and probe for the IC target sequence are configured and synthesized by using any well-known method provided that the primers and probe function for amplification of the IC target sequence and detection of the amplified IC sequence using substantially the same assay conditions used to amplify and detect the human adenovirus target sequence. In certain embodiments that include a target capture-based purification step, it is preferred that a target capture probe specific for the IC target be included in the assay in the target capture step so that the IC is treated in the assay in a manner analogous to that for the intended human adenovirus analyte in all of the assay steps.

### Preparation of Oligonucleotides

The amplification oligomers and probes of the present disclosure can be readily prepared by methods known in the art. Preferably, the oligonucleotides are synthesized using solid phase methods. Standard phosphoramidite solid-phase chemistry for joining nucleotides by phosphodiester linkages is disclosed by Caruthers et al., in Chemical Synthesis of Deoxynucleotides by the Phosphoramidite Method. Methods Enzymol., 154:287 (1987). Automated solid-phase chemical synthesis using cyanoethyl phosphoramidite precursors has been described by Barone. See Barone et al., In Situ Activation of bis-dialkylaminephosphines—a New Method for Synthesizing Deoxyoligonucleotides on Polymer Supports, Nucleic Acids Res., 12(10):4051 (1984). Batt discloses a procedure for synthesizing oligonucleotides containing phosphorothioate linkages in U.S. Pat. No. 5,449,769, entitled Method and Reagent for Sulfurization of Organophosphorous Compounds. In addition. Riley et al. disclose the synthesis of oligonucleotides having different linkages including methylphosphonate linkages in U.S. Pat. No. 5,811,538, titled Process for the Purification of Oligomers. Moreover, methods for the organic synthesis of oligonucleotides are known to those of skill in the art and are described in, for example, Sambrook, Fritsch & Maniatis, Molecular Cloning: A Laboratory Manual, Second Edition, Cold Spring Harbor, N.Y.: Cold Spring Harbor Laboratory Press, 1989 (herein “Sambrook et al.), ch. 10.

Following synthesis and purification of a particular oligonucleotide, several different procedures may be utilized to purify and control the quality of the oligonucleotide. Suitable procedures include polyacrylamide gel electrophoresis or high pressure liquid chromatography. Both of these procedures are well known to those skilled in the art.

All of the oligonucleotides of the present disclosure, whether amplification oligomers or probes, may be modified with chemical groups to enhance their performance or to facilitate the characterization of amplification products. For example, backbone-modified oligonucleotides such as those having phosphorothioate, methylphosphonate, 2′-O-alkyl or peptide groups which render the oligonucleotides resistant to the nucleolytic activity of certain polymerases or to nuclease enzymes may allow the use of such enzymes in an amplification or other reaction. Another example of a modification involves using non-nucleotide linkers incorporated between nucleotides in the nucleic acid chain of a probe or primer, and which do not prevent hybridization of a probe or hybridization and elongation of a primer. See Arnold et al., Non-Nucleotide Linking Reagents for Nucleotide Probes. U.S. Pat. No. 6,031,091. The oligonucleotides of the present disclosure may also contain mixtures of the desired modified and natural nucleotides.

The 3′ end of an amplification oligonucleotide can be modified or blocked to prevent or inhibit initiation of DNA synthesis, as disclosed by Kacian et al, in U.S. Pat. No. 5,554,516. The 3′ end of the amplification oligonucleotide can be modified in a variety of ways well-known in the art. By way of example, appropriate modifications can include the addition of ribonucleotides, 3′ deoxynucleotide residues (e.g., cordycepin), 2′,3′-dideoxynucleotide residues, modified nucleotides such as phosphorothioates, and non-nucleotide linkages such as those disclosed by Arnold et al, in U.S. Pat. No. 6,031,091 or alkane-diol modifications (see Wilk et al., Backbone-Modified Oligonucleotides Containing a Butanediol-1,3 Moiety as a Vicarious Segment for the Deoxyribosyl Moiety—Synthesis and Enzyme Studies, Nucleic Acids Res., 18(8): 2065 (1990)), or the modification may simply consist of a region 3′ to the priming sequence that is non-complementary to the target nucleic acid sequence. Additionally, a mixture of different 3′ blocked primers or of 3′ blocked and unblocked primers may increase the efficiency of nucleic acid amplification, as disclosed therein.

The 5′ end of primers can be modified to be resistant to the 5′-exonuclease activity present in some nucleic acid polymerases. Such modifications can be carried out by adding a non-nucleotide group to the terminal 5′ nucleotide of the primer using techniques such as those disclosed by Arnold et al, in U.S. Pat. No. 6,031,091. To facilitate strand displacement, the 5′ end may also be modified to include non-complementary nucleotides as disclosed by Dattagupta et al, Isothermal Strand Displacement Nucleic Acid Amplification. U.S. Pat. No. 6,087,133.

Once synthesized, a selected oligonucleotide may be labeled by any of several well-known methods (see, e.g., Sambrook et al., supra, ch. 10). Useful labels include radioisotopes as well as non-radioactive reporting groups. Isotopic labels include ³H, ³⁵S, ³²P, ¹²⁵I, ⁵⁷Co and ¹⁴C. Isotopic labels can be introduced into the oligonucleotide by techniques known in the art such as nick translation, end labeling, second strand synthesis, the use of reverse transcription, and by chemical methods. When using radiolabeled probes, hybridization can be detected by autoradiography, scintillation counting or gamma counting. The detection method selected will depend upon the particular radioisotope used for labeling.

Non-isotopic materials can also be used for labeling and may be introduced internally into the nucleic acid sequence or at the end of the nucleic acid sequence. Modified nucleotides may be incorporated enzymatically or chemically. Chemical modifications of the probe may be performed during or after synthesis of the probe, for example, through the use of non-nucleotide linker groups, as disclosed by Arnold et al, in U.S. Pat. No. 6,031,091. Non-isotopic labels include fluorescent molecules (individual labels or combinations of interacting labels, such as the fluorescence resonance energy transfer (FRET) pairs disclosed by Tyagi et al, in U.S. Pat. No. 5,925,517), chemiluminescent molecules, enzymes, cofactors, enzyme substrates, haptens, or other ligands. In some embodiments, detection probes of the present disclosure are labeled by means of a non-nucleotide linker with an acridinium ester (AE), such as standard AE. Acridinium ester labeling can be performed as disclosed by Arnold et al., Acridinium Ester Labelling and Purification of Nucleotide Probes, U.S. Pat. No. 5,185,439.

### Formulations, Reaction Mixtures, and Kits

Also provided herein are formulations for determining the presence or absence of human adenovirus (HAdV) in a sample. In some embodiments, a formulation is an aqueous formulation comprising (1) at least two HAdV-specific amplification oligomers for amplification of an human adenovirus target region as described herein and (2) an organic buffer. An aqueous formulation for amplification of a human adenovirus nucleic acid may include one or more additional components such as, e.g., a DNA polymerase enzyme, a reverse transcriptase enzyme, or a detection probe oligomer. In some embodiments, a formulation is an aqueous formulation comprising (1) a HAdV-specific detection probe oligomer as described herein and (2) an organic buffer. An aqueous formulation comprising one or more detection probe oligomers may include one or more additional components such as, e.g., a surfactant, a DNA polymerase enzyme, a reverse transcriptase enzyme, or at least one amplification oligomer. Particularly suitable surfactants include, for example, polyethylene glycol mono [4-(1,1,3,3-tetramethylbutyl) phenyl] ether and polyoxyethylene sorbitan fatty acid esters (e.g., polysorbate 20, polysorbate 40, or polysorbate 60). In some embodiments, a surfactant in an aqueous detection probe formulation is a non-linear surfactant (i.e., a surfactant having a branched chain structure) such as, for example, a polyoxyethylene sorbitan fatty acid ester (e.g., polysorbate 20, polysorbate 40, or polysorbate 60) or digitonin. An aqueous formulation as above for amplification or detection of human adenovirus nucleic acid may further include a bulking agent such as, e.g., trehalose, raffinose, or a combination thereof. In some embodiments, an aqueous formulation as above contains at inorganic salt such as, e.g., magnesium, potassium, or sodium; in some such variations, the concentration of the inorganic salt is 4 mM or less. A particularly suitable organic buffer for an aqueous formulation as above is Tris (2-amino-2-(hydroxymethyl)-1,3-propanediol).

In a related aspect, for long-term storage, an aqueous formulation as described herein may be aliquoted into, e.g., vials, ampules, or other containers and dried (e.g., lyophilized) according to procedures known in the art. The dried product typically appears as a powder or cake. The containers are then sealed. Methods of preparing such dried formulations from the aqueous formulation, as well as the dried formulations prepared by such methods, are additional aspects of the instant disclosure. In yet another aspect, there is provided a dried formulation that enables reconstitution into an aqueous formulation as described herein. Dried formulations for amplification or detection of a human adenovirus nucleic acid typically contain, in addition to one or more amplification oligomers and/or detection probes as described herein, a bulking agent such as, e.g., trehalose, raffinose, or a combination thereof. In some embodiments further comprising an inorganic salt, the percent mass of the inorganic salt to the mass of the dried formulation is 0.249% or less. 0.222% or less, or 0.195% or less. Methods of preparing a dried formulation from a lyophilized formulation as described herein are also encompassed by the instant disclosure; such methods generally include dissolving the dried formulation in a suitable diluent (e.g., an organic buffer or water) to provide a reconstituted formulation.

Also provided is a reaction mixture for determining the presence or absence of an human adenovirus target nucleic acid in a sample. A reaction mixture in accordance with the present disclosure includes one or both of (1) an oligomer combination as described herein for amplification of a human adenovirus target nucleic acid and (2) one or more detection probe oligomers as described herein for determining the presence or absence of a human adenovirus amplification product. The reaction mixture may further include several optional components such as, for example, capture probes, e.g., poly-(k) capture probes as described in US 2013/0209992, which is incorporated herein by reference. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components, in which a human adenovirus target nucleic acid may or may not be present. A reaction mixture may include amplification oligomers for only one target region of a human adenovirus genome, or it may include amplification oligomers for multiple human adenovirus target regions. In addition, for a reaction mixture that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture). In some embodiments, a reaction mixture comprises an aqueous formulation as described above. In some embodiments, a reaction mixture is reconstituted with water or an organic buffer from a dried formulation as described above.

Also provided herein are kits for practicing the methods as described herein. A kit in accordance with the present disclosure includes one or both of (1) an oligomer combination as described herein for amplification of an human adenovirus target nucleic acid and (2) one or more detection probe oligomers as described herein for determining the presence or absence of an human adenovirus amplification product. The kits may further include several optional components such as, for example, capture probes, e.g., poly-(k) capture probes as described in US 2013/0209992. Other reagents that may be present in the kits include reagents suitable for performing in vitro amplification such as, e.g., buffers, salt solutions, appropriate nucleotide triphosphates (e.g., dATP, dCTP, dGTP, dTTP; and/or ATP, CTP, GTP and UTP), and/or enzymes (e.g., a thermostable DNA polymerase, or a reverse transcriptase and/or RNA polymerase). Oligomers as described herein may be packaged in a variety of different embodiments, and those skilled in the art will appreciate that the disclosure embraces many different kit configurations. For example, a kit may include amplification oligomers for only one target region of a human adenovirus genome, or it may include amplification oligomers for multiple human adenovirus target regions. In addition, for a kit that includes a detection probe together with an amplification oligomer combination, selection of amplification oligomers and detection probe oligomers for a kit are linked by a common target region (i.e., the kit will include a probe that binds to a sequence amplifiable by an amplification oligomer combination of the kit). In certain embodiments, the kit further includes a set of instructions for practicing methods in accordance with the present disclosure, where the instructions may be associated with a package insert and/or the packaging of the kit or the components thereof.

The compositions, kits, formulations, reaction mixtures, and methods are further illustrated by the following non-limiting examples.

### EXAMPLES

### Example 1

This example describes screening of two primer probe combinations (“Multiplex 1” and “Multiplex 2” oligos) against live adenoviruses A, B, C, D, E, and F.

1.25× primer and probe mixtures (PPR mixes) were prepared in a microfuge tube to include three forward primers, four reverse primers, and two HEX-labeled labeled detection probes for each of the Multiplex 1 and Multiplex 2 oligo combinations. The internal control PPR mix comprised 0.3125 μM of each primer and 0.25 μM of the probe, while the adenovirus PPR mixes contained 0.75 μM of each primer and 0.5 μM of each probe. These PPR mixes also contained 81.25 mM of KCl, 5 mM MgCl₂, and were brought to final volume in water. The combinations of primers and probes for Multiplexes 1 and 2 are shown in Table 1.

TABLE 1

| | | | SEQ | |
|---|---|---|---|---|
| | | | ID | Sequence |
| Component | Oligo | Multiplex | NO | (5′ to 3′) |
| Forward Primer | HAdV Penton FP 1 | 1, 2 | 3 | ACCGTCAGTGAAAACGT |
| Forward Primer | HAdV Penton FP 2 | 1, 2 | 4 | ACCGTTAGTGAAAACGT |
| Forward Primer | HAdV Penton FP 3 | 1, 2 | 5 | ACCATCAGTGAAAACGT |
| Reverse Primer | HAdV Penton RP 1 | 1 | 6 | GCCTTGTAGACGTAGGG |
| Reverse Primer | HAdV Penton RP 2 | 1 | 7 | GCCTTGTAAACGTAGGG |
| Reverse Primer | HAdV Penton RP 3 | 1 | 8 | GCTTTGTACACGTAAGG |
| Reverse Primer | HAdV Penton RP 4 | 1 | 9 | GCTTTGTGGACGTAGGG |
| Reverse Primer | HAdV Penton RP 5 | 2 | 10 | TCACTCGCTGGACTC |
| Reverse Primer | HAdV Penton RP 6 | 2 | 11 | TCACGCGCTGGACTC |
| Reverse Primer | HAdV Penton RP 7 | 2 | 12 | TCACACGTTGGACTC |
| Reverse Primer | HAdV Penton RP 8 | 2 | 13 | TCACGCGCTGAACTC |
| Probe | HAdV Penton PR 1 | 1, 2 | 15 | HEX-CCTGCTCTCACAGATCACGGGAC-BHQ1 |
| Probe | HAdV Penton PR 2 | 1, 2 | 16 | HEX-CCCGCCCTCACAGATCACGGAAC-BHQ1 |

Note:
Underlined C = 5-methyl cytosine

For each of human adenovirus serotypes 31, 11, 2, 8, 4, and 41 (representing subtypes A, B, C, D, E, and F, respectively), a separate viral culture stock mix in sample transport medium (STM; containing LLS, EDTA, and sodium phosphate) was prepared from a stock concentration to provide a concentration of 10 TCID50 per reaction (278 TCID50/mL). Samples were lysed to release target nucleic acids from the virus types. The released target nucleic acids were purified and contacted with amplification and detection reaction mixture containing a PPR mix according to the example.

Amplification and detection reactions were set up at 6 reactions per condition; 3 reactions positive for the human adenovirus subtype and 3 reactions negative for the subtype. Negative reactions included sample transport media without adenovirus. The reactions were performed as real-time PCR reactions using a Panther Fusion system (available from Hologic, Inc., Marlborough MA). Results are shown in Table 2 below.

TABLE 2

| | | | Background | |
|---|---|---|---|---|
| Sample | CT | RFU | RFU | Noise |
| HAdV A | 26.97 | 19702 | 15731 | 1.25 |
| Multiplex 1 | 27.06 | 20481 | 16945 | 1.21 |
| | 27.35 | 16583 | 13006 | 1.28 |
| HAdV A | 22.35 | 21504 | 12515 | 1.72 |
| Multiplex 2 | 21.91 | 25817 | 15028 | 1.72 |
| | 22.02 | 24376 | 14492 | 1.68 |
| HAdV B | 24.91 | 16244 | 13527 | 1.20 |
| Multiplex 1 | 24.77 | 16680 | 14010 | 1.19 |
| | 24.22 | 19757 | 16957 | 1.17 |
| HAdV B | 24.7 | 18232 | 15788 | 1.15 |
| Multiplex 2 | 24.44 | 18801 | 16226 | 1.16 |
| | 24.24 | 19109 | 16679 | 1.15 |
| HAdV C | 29.21 | 18521 | 17573 | 1.05 |
| Multiplex 1 | 29.01 | 17030 | 16362 | 1.04 |
| | 29.62 | 16635 | 15014 | 1.11 |
| HAdV C | 28.06 | 18893 | 17624 | 1.07 |
| Multiplex 2 | 28.71 | 16125 | 13554 | 1.19 |
| | 29.13 | 13202 | 10973 | 1.20 |
| HAdV D | 23.3 | 16566 | 13058 | 1.27 |
| Multiplex 1 | 23.37 | 17868 | 14828 | 1.21 |
| | 23.02 | 20010 | 16177 | 1.24 |
| HAdV D | 23.73 | 17611 | 14160 | 1.24 |
| Multiplex 2 | 23.73 | 18476 | 15110 | 1.22 |
| | 23.96 | 15822 | 12886 | 1.23 |
| HAdV E | 20.01 | 19260 | 15107 | 1.27 |
| Multiplex 1 | 20.88 | 14150 | 11128 | 1.27 |
| | 20.49 | 18500 | 14169 | 1.31 |
| HAdV E | 20.61 | 19368 | 17200 | 1.13 |
| Multiplex 2 | 21.19 | 18211 | 15378 | 1.18 |
| | 21.44 | 15447 | 13923 | 1.11 |
| HAdV F | 22.64 | 28809 | 16825 | 1.71 |
| Multiplex 1 | 22.53 | 26683 | 15524 | 1.72 |
| | 23.09 | 23903 | 14026 | 1.70 |
| HAdV F | 23.18 | 22990 | 13026 | 1.76 |
| Multiplex 2 | 22.77 | 27118 | 14964 | 1.81 |
| | 23.03 | 26131 | 15020 | 1.74 |

The results show that both multiplexes performed well for detection of all six adenovirus subtypes.

### Example 2

This example describes screening of two primer probe combinations (Multiplex 1 and Multiplex 2 oligos from Example 1) against penton gene plasmids for adenovirus subtypes A, B, C, D, E, and F.

1.25× primer and probe mixtures (PPR mixes) were prepared as described in Example 1.

For each of human adenovirus serotypes 31, 11, 2, 8, 4, and 41 (representing subtypes (A, B, C, D, E, and F, respectively), HAdV plasmid stock was diluted in STM to provide HAdV plasmids at 1,000 copies/reaction.

Amplification and detection reactions were set up at 6 reactions per condition; 3 reactions positive for the human adenovirus subtype and 3 reactions negative for the subtype. Negative reactions included sample transport media without adenovirus. The reactions were performed as a real-time PCR reactions using a Panther Fusion system (available from Hologic, Inc., Marlborough MA). Results are shown in Tables 3 and 4 below.

TABLE 3

| | | | Background | |
|---|---|---|---|---|
| Sample | CT | RFU | RFU | Noise |
| HAdV A | 31.62 | 15404 | 13841 | 1.11 |
| Multiplex 1 | 31.74 | 14974 | 12856 | 1.16 |
| | 31.61 | 16130 | 13964 | 1.16 |
| HAdV A | 31.11 | 18317 | 12918 | 1.42 |
| Multiplex 2 | 31.18 | 18796 | 13223 | 1.42 |
| | 31.44 | 18181 | 14095 | 1.29 |
| HAdV B | 28.92 | 20285 | 19094 | 1.06 |
| Multiplex 1 | 29.21 | 17750 | 15457 | 1.15 |
| | 29.61 | 15695 | 14457 | 1.09 |
| HAdV B | 30.57 | 14417 | 14034 | 1.03 |
| Multiplex 2 | 30.57 | 14480 | 13050 | 1.11 |
| | 30.1 | 17296 | 14826 | 1.17 |
| HAdV C | 29.17 | 18359 | 17365 | 1.06 |
| Multiplex 1 | 29.32 | 18265 | 15128 | 1.21 |
| | 29.68 | 15375 | 13438 | 1.14 |
| HAdV C | 29.42 | 17068 | 14791 | 1.15 |
| Multiplex 2 | 29.36 | 18305 | 15222 | 1.20 |
| | 29.91 | 14766 | 12426 | 1.19 |
| HAdV D | 29.93 | 19235 | 16779 | 1.15 |
| Multiplex 1 | 30.68 | 15364 | 13255 | 1.16 |
| | 29.93 | 19313 | 16685 | 1.16 |
| HAdV D | 31.03 | 17611 | 16613 | 1.06 |
| Multiplex 2 | 30.58 | 18501 | 16882 | 1.10 |
| | 30.59 | 18942 | 16329 | 1.16 |
| HAdV E | 30.08 | 16842 | 15136 | 1.11 |
| Multiplex 1 | 29.52 | 19231 | 16869 | 1.14 |
| | 30.22 | 16988 | 14458 | 1.17 |
| HAdV E | 29.82 | 18216 | 15193 | 1.20 |
| Multiplex 2 | 29.95 | 17838 | 14466 | 1.23 |
| | 30.21 | 16302 | 13297 | 1.23 |
| HAdV F | 29.03 | 21443 | 15608 | 1.37 |
| Multiplex 1 | 29.41 | 17846 | 11819 | 1.51 |
| | 29.15 | 20535 | 13903 | 1.48 |
| HAdV F | 29.03 | 19787 | 13438 | 1.47 |
| Multiplex 2 | 29.28 | 19191 | 13949 | 1.38 |
| | 28.98 | 20386 | 14646 | 1.39 |

TABLE 4

| Adenovirus | Multiplex | Average | | Quantification |
|---|---|---|---|---|
| Subtype | Design | CT | SD | Potential* |
| A | 1 | 31.66 | 0.07 | − |
| | 2 | 31.24 | 0.17 | − |
| B | 1 | 29.25 | 0.35 | ++ |
| | 2 | 30.41 | 0.27 | − |
| C | 1 | 29.39 | 0.26 | ++ |
| | 2 | 29.56 | 0.30 | ++ |
| D | 1 | 30.18 | 0.43 | + |
| | 2 | 30.73 | 0.26 | − |
| E | 1 | 29.94 | 0.37 | + |
| | 2 | 29.99 | 0.20 | + |
| F | 1 | 29.20 | 0.19 | ++ |
| | 2 | 29.10 | 0.16 | ++ |

*Possible quantification potential based on average CTs for all subtypes;
++ = good;
+ = not ideal but can be optimized.
To be quantitative, it is preferred that the CTs of the same concentration for all subtypes be within 2.32.

The data suggest that Multiplex 1 is better than Multiplex 2 since it has better quantifiable possibilities. The data show that subtype A is not in the quantifiable range, so this adenovirus subtype will likely need an addition primer or probe to bring in within quantifiable range.

### Example 3

The objective of this study is to observe the detection all the adenovirus subtypes using the Multiplex 1 and Multiplex 2 combinations with the addition of a third probe, which is specific to subtype A. Subtype A is detected with the latest CT values using the Multiplex 1 and Multiplex 2 primer probe combinations in Examples 1 and 2 above. The goal is to improve adenovirus A detection while not negatively affecting the performance of the other subgroups.

Four different 1.25× primer and probe mixtures (PPR mixes) were prepared in a microfuge tube to include three forward primers, four reverse primers, and either two or three detection probes: one PPR mix for each of the Multiplex 1 and Multiplex 2 oligo combinations as described in Example 1, and one PPR mix for each of Multiplex 1 and Multiplex 2 further containing a third probe (HEX-CCCGCTCTCACAGATCACGGGAC-BHQ1; SEQ ID NO:17). The internal control PPR mix comprised 0.75 μM of each primer and 0.5 μM of the probe, while the adenovirus PPR mixes contained 0.75 μM of each primer and 0.375 μM of each probe. These PPR mixes also contained 81.25 mM of KCl, 5 mM MgCl₂, and were brought to final volume in water.

For each of human adenovirus serotypes 18, 11, 5, 8, 4, and 41 (representing subtypes A, B, C, D, E, and F, respectively), HAdV plasmid stock was diluted in STM to provide HAdV plasmids at 1,000 copies/reaction.

Amplification and detection reactions were set up at 6 reactions per condition; 3 reactions positive for the human adenovirus subtype and 3 reactions negative for the subtype. Negative reactions included sample transport media without adenovirus. The reactions were performed as real-time PCR reactions using a Panther Fusion system (available from Hologic, Inc., Marlborough MA). Results are shown in Tables 5 and 6 below.

TABLE 5

| | | | Background | |
|---|---|---|---|---|
| Sample | CT | RFU | RFU | Noise |
| HAdV A | 34.11 | 11759 | 11641 | 1.01 |
| Multiplex 1 | 34.58 | 10175 | 8929 | 1.14 |
| 2 Probes | 33.74 | 12757 | 11844 | 1.08 |
| HAdV A | 31.67 | 17554 | 19057 | 0.92 |
| Multiplex 1 | 32.18 | 15552 | 17574 | 0.88 |
| 3 Probes | 31.14 | 17726 | 19978 | 0.89 |
| HAdV A | 34.18 | 15386 | 13839 | 1.11 |
| Multiplex 2 | 34.49 | 13335 | 12723 | 1.05 |
| 2 Probes | 34.62 | 14054 | 13160 | 1.07 |
| HAdV A | 33.18 | 17893 | 18832 | 0.95 |
| Multiplex 2 | 33.42 | 13728 | 13757 | 1.00 |
| 3 Probes | 33.68 | 13243 | 13321 | 0.99 |
| HAdV B | 32.09 | 13788 | 12180 | 1.13 |
| Multiplex 1 | 32.85 | 10236 | 9730 | 1.05 |
| 2 Probes | 33.16 | 9807 | 8625 | 1.14 |
| HAdV B | 31.10 | 18927 | 17367 | 1.09 |
| Multiplex 1 | 30.77 | 20586 | 19831 | 1.04 |
| 3 Probes | 31.83 | 15428 | 12889 | 1.20 |
| HAdV B | 33.13 | 12951 | 13045 | 0.99 |
| Multiplex 2 | 33.13 | 13076 | 13017 | 1.00 |
| 2 Probes | 33.31 | 12568 | 12801 | 0.98 |
| HAdV B | 33.11 | 13378 | 12596 | 1.06 |
| Multiplex 2 | 32.22 | 17982 | 18330 | 0.98 |
| 3 Probes | 32.41 | 15339 | 15134 | 1.01 |
| HAdV C | 31.09 | 11815 | 9973 | 1.18 |
| Multiplex 1 | 30.45 | 14230 | 12399 | 1.15 |
| 2 Probes | 31.01 | 12675 | 11018 | 1.15 |
| HAdV C | 30.31 | 19976 | 17422 | 1.15 |
| Multiplex 1 | 30.36 | 18696 | 16599 | 1.13 |
| 3 Probes | 30.87 | 18346 | 15950 | 1.15 |
| HAdV C | 30.82 | 14844 | 13445 | 1.10 |
| Multiplex 2 | 30.96 | 14544 | 14177 | 1.03 |
| 2 Probes | 31.16 | 11986 | 10034 | 1.19 |
| HAdV C | 30.21 | 19557 | 14846 | 1.32 |
| Multiplex 2 | 30.12 | 20368 | 15147 | 1.34 |
| 3 Probes | 30.29 | 19974 | 14665 | 1.36 |
| HAdV D | 32.46 | 12111 | 10727 | 1.13 |
| Multiplex 1 | 32.39 | 12814 | 11263 | 1.14 |
| 2 Probes | 32.43 | 11873 | 9961 | 1.19 |
| HAdV D | 31.63 | 18028 | 17588 | 1.03 |
| Multiplex 1 | 31.55 | 16634 | 15939 | 1.04 |
| 3 Probes | 31.56 | 17224 | 15527 | 1.11 |
| HAdV D | 32.43 | 11244 | 9142 | 1.23 |
| Multiplex 2 | 32.45 | 14558 | 12563 | 1.16 |
| 2 Probes | 31.69 | 16493 | 14592 | 1.13 |
| HAdV D | 32.31 | 17839 | 16161 | 1.10 |
| Multiplex 2 | 31.28 | 22788 | 19475 | 1.17 |
| 3 Probes | 31.35 | 22804 | 20449 | 1.12 |
| HAdV E | 32.37 | 11945 | 10927 | 1.09 |
| Multiplex 1 | 31.86 | 14004 | 12825 | 1.09 |
| 2 Probes | 31.90 | 13841 | 12146 | 1.14 |
| HAdV E | 30.94 | 19888 | 17721 | 1.12 |
| Multiplex 1 | 31.75 | 14586 | 13798 | 1.06 |
| 3 Probes | 31.57 | 16033 | 13468 | 1.19 |
| HAdV E | 31.51 | 15197 | 12987 | 1.17 |
| Multiplex 2 | 31.69 | 14917 | 12433 | 1.20 |
| 2 Probes | 31.94 | 13395 | 11112 | 1.21 |
| HAdV E | 31.77 | 17977 | 17091 | 1.05 |
| Multiplex 2 | 31.93 | 19771 | 17987 | 1.10 |
| 3 Probes | 32.07 | 20224 | 18473 | 1.09 |
| HAdV F | 30.81 | 15121 | 10269 | 1.47 |
| Multiplex 1 | 30.59 | 17867 | 11601 | 1.54 |
| 2 Probes | 31.11 | 15198 | 10311 | 1.47 |
| HAdV F | 29.91 | 22197 | 16696 | 1.33 |
| Multiplex 1 | 30.01 | 20783 | 14681 | 1.42 |
| 3 Probes | 30.02 | 21230 | 15093 | 1.41 |
| HAdV F | 30.74 | 15866 | 11070 | 1.43 |
| Multiplex 2 | 30.83 | 14489 | 10079 | 1.44 |
| 2 Probes | 30.63 | 16015 | 10902 | 1.47 |
| HAdV F | 30.66 | 18395 | 16223 | 1.13 |
| Multiplex 2 | 29.72 | 24364 | 19984 | 1.22 |
| 3 Probes | 30.49 | 19149 | 16894 | 1.13 |

TABLE 6

| Adenovirus | Multiplex | | Average | |
|---|---|---|---|---|
| Subtype | Design | # Probes | CT* | SD |
| A | 1 | 2 | 34.14 | 0.42 |
| | | 3 | 31.66 | 0.52 |
| | 2 | 2 | 34.43 | 0.23 |
| | | 3 | 33.43 | 0.25 |
| B | 1 | 2 | 32.70 | 0.55 |
| | | 3 | 31.23 | 0.54 |
| | 2 | 2 | 33.19 | 0.10 |
| | | 3 | 32.58 | 0.47 |
| C | 1 | 2 | 30.85 | 0.35 |
| | | 3 | 30.51 | 0.31 |
| | 2 | 2 | 30.98 | 0.17 |
| | | 3 | 30.21 | 0.09 |
| D | 1 | 2 | 32.43 | 0.04 |
| | | 3 | 31.58 | 0.04 |
| | 2 | 2 | 32.19 | 0.43 |
| | | 3 | 31.65 | 0.58 |
| E | 1 | 2 | 32.04 | 0.28 |
| | | 3 | 31.42 | 0.43 |
| | 2 | 2 | 31.71 | 0.22 |
| | | 3 | 31.92 | 0.15 |
| F | 1 | 2 | 30.84 | 0.26 |
| | | 3 | 29.98 | 0.06 |
| | 2 | 2 | 30.73 | 0.10 |
| | | 3 | 30.29 | 0.50 |

*CT values in bold type indicate best result for subtype

The Multiplex 1 design with the third probe performed the best: with all subtypes except HAdV C, the best CTs were observed in Multiplex 1 with three probes; the second best option for HAdV C was Multiplex 1 with three probes. Additionally, all subtypes appear to be within 2.32 CTs from each other with this option, which is within the range for quantification (half a log is 2.32 CTs). Multiplex 1 with three probes will be used for further development.

### Example 4

The objective of this study is to check if the presence of HeLa cells as a mock matrix as any effect on the detection of the penton gene plasmids for adenovirus subtypes A, B, C, D, E, and F.

1.25× primer and probe mixtures (PPR mixes) were prepared in a microfuge tube to include the oligonucleotides of Multiplex 1 containing three probes as described in Example 3. The internal control PPR mix comprised 0.75 μM of each primer and 0.5 μM of the probe, while the adenovirus PPR mixes contained 0.75 μM of each primer and 0.375 μM of each probe. These PPR mixes also contained 81.25 mM of KCl, 5 mM MgCl₂, and were brought to final volume in water.

For each of human adenovirus serotypes 18, 11, 5, 8, 4, and 41 (representing subtypes A, B, C, D, E, and F, respectively), HAdV plasmid stock was diluted in STM, or STM supplemented with 10,000 HeLa cells/mL, to provide HAdV plasmids at 1,000 copies/reaction.

Amplification and detection reactions were set up at 6 reactions per condition; 3 reactions positive for the human adenovirus subtype and 3 reactions negative for the subtype. Negative reactions included sample transport media without adenovirus. The reactions were performed using a Panther Fusion system (available from Hologic, Inc., Marlborough MA). Results are shown in Tables 7 and 8 below.

TABLE 7

| | | | Background | |
|---|---|---|---|---|
| Sample | CT | RFU | RFU | Noise |
| HAdV A − | 33.36 | 12279 | 17949 | 0.68 |
| HeLa | 32.41 | 15777 | 22358 | 0.71 |
| | 33.93 | 11357 | 18539 | 0.61 |
| HAdV A + | 33.16 | 14159 | 20043 | 0.71 |
| HeLa | 34.20 | 11840 | 18284 | 0.65 |
| | 33.05 | 13911 | 20581 | 0.68 |
| HAdV B − | 32.06 | 16443 | 17711 | 0.93 |
| HeLa | 31.64 | 17923 | 19044 | 0.94 |
| | 31.30 | 18573 | 20486 | 0.91 |
| HAdV B + | 31.52 | 16244 | 17550 | 0.93 |
| HeLa | 31.78 | 18552 | 19844 | 0.93 |
| | 31.35 | 18936 | 19132 | 0.99 |
| HAdV C − | 29.63 | 23349 | 22868 | 1.02 |
| HeLa | 30.56 | 19874 | 18542 | 1.07 |
| | 30.21 | 23224 | 22445 | 1.03 |
| HAdV C + | 30.74 | 22135 | 22069 | 1.00 |
| HeLa | 30.64 | 21387 | 24698 | 0.87 |
| | 30.55 | 20494 | 21963 | 0.93 |
| HAdV D − | 31.74 | 17543 | 18207 | 0.96 |
| HeLa | 31.15 | 19715 | 20898 | 0.94 |
| | 31.79 | 17168 | 17287 | 0.99 |
| HAdV D + | 32.56 | 16909 | 19990 | 0.85 |
| HeLa | 32.1 | 18233 | 20793 | 0.88 |
| | 32.53 | 16816 | 18817 | 0.89 |
| HAdV E − | 31.17 | 18943 | 21689 | 0.87 |
| HeLa | 32.7 | 16478 | 18069 | 0.91 |
| | 32.47 | 17019 | 17871 | 0.95 |
| HAdV E + | 32.07 | 17933 | 19188 | 0.93 |
| HeLa | 32.39 | 16772 | 18925 | 0.89 |
| | 32.4 | 16013 | 16693 | 0.96 |
| HAdV F − | 30.28 | 21847 | 18885 | 1.16 |
| HeLa | 30.43 | 20658 | 16953 | 1.22 |
| | 30.33 | 21010 | 17227 | 1.22 |
| HAdV F + | 31.36 | 16481 | 15504 | 1.06 |
| HeLa | 30.49 | 22260 | 19506 | 1.14 |
| | 30.78 | 21770 | 20392 | 1.07 |

TABLE 8

| Adenovirus | HeLa | Average | |
|---|---|---|---|
| Subtype | Cells | CT | SD |
| A | No | 33.23 | 0.77 |
| | Yes | 33.47 | 0.63 |
| B | No | 31.67 | 0.38 |
| | Yes | 31.55 | 0.22 |
| C | No | 30.13 | 0.47 |
| | Yes | 30.64 | 0.10 |
| D | No | 31.56 | 0.36 |
| | Yes | 32.40 | 0.26 |
| E | No | 32.11 | 0.83 |
| | Yes | 32.29 | 0.19 |
| F | No | 30.35 | 0.08 |
| | Yes | 30.88 | 0.44 |

All adenovirus subtypes show similar CTs within one (1) CT between positive and negative HeLa matrices, indicating that there is little to no oligo interactions with human genomic DNA.

### Example 5

The objective of this study is to confirm that the addition of Hela cells to STM will still result in the detection of penton gene plasmids for adenovirus subtypes A, B, C, D, E, and F at 50 copies/reaction.

1.25× primer and probe mixtures (PPR mixes) were prepared in a microfuge tube to include the oligonucleotides of Multiplex 1 containing three probes as described in Example 3. The internal control PPR mix comprised 0.75 μM of each primer and 0.5 μM of the probe, while the adenovirus PPR mixes contained 0.75 μM of each primer and 0.375 μM of each probe. These PPR mixes also contained 81.25 mM of KCl, 5 mM MgCl₂, and were brought to final volume in water.

For each of human adenovirus serotypes 18, 11, 5, 8, 4, and 41 (representing subtypes A, B, C, D, E, and F, respectively), HAdV plasmid stock was diluted in STM, or STM supplemented with 20,000 HeLa cells/mL, to provide HAdV plasmids at 500 copies/reaction or 50 copies/reaction.

Amplification and detection reactions were set up at 6 reactions per condition; 3 reactions positive for the human adenovirus subtype and 3 reactions negative for the subtype. Negative reactions included sample transport media without adenovirus. The reactions were performed as real-time PCR reactions using a Panther Fusion system (available from Hologic, Inc., Marlborough MA). Results are shown in Tables 9-12 below.

TABLE 9

| Sample | CT | RFU | Background RFU | Noise |
|---|---|---|---|---|
| HAdV A | 35.82 | 10315 | 20588 | 0.50 |
| 500 copies | 35.11 | 12464 | 20819 | 0.60 |
| | 34.73 | 11819 | 22804 | 0.52 |
| HAdV A | 40.8 | 2702 | 17536 | 0.15 |
| 50 copies | 40.44 | 3265 | 21086 | 0.15 |
| | 41.55 | 2085 | 18487 | 0.11 |
| HAdV B | 34.88 | 13432 | 22632 | 0.59 |
| 500 copies | 34.61 | 13599 | 23535 | 0.58 |
| | 34.44 | 14958 | 21907 | 0.68 |
| HAdV B | 39.73 | 4398 | 17471 | 0.25 |
| 50 copies | 38.62 | 6174 | 19739 | 0.31 |
| | 39.42 | 4505 | 18881 | 0.24 |
| HAdV C | 31.96 | 19571 | 22428 | 0.87 |
| 500 copies | 32.25 | 17619 | 21046 | 0.84 |
| | 32.15 | 16652 | 20091 | 0.83 |
| HAdV C | 35.76 | 10403 | 17320 | 0.60 |
| 50 copies | 37.23 | 7449 | 15317 | 0.49 |
| | 37.64 | 6798 | 14120 | 0.48 |
| HAdV D | 35.25 | 11399 | 17988 | 0.63 |
| 500 copies | 35.38 | 12007 | 21310 | 0.56 |
| | 35.36 | 12178 | 19776 | 0.62 |
| HAdV D | 40.09 | 4223 | 20611 | 0.20 |
| 50 copies | 38.9 | 5625 | 21325 | 0.26 |
| | 38.06 | 7079 | 19918 | 0.36 |
| HAdV E | 35.01 | 14570 | 22461 | 0.65 |
| 500 copies | 34.7 | 14993 | 21915 | 0.68 |
| | 35.64 | 11086 | 20299 | 0.55 |
| HAdV E | 35.96 | 11771 | 22597 | 0.52 |
| 50 copies | 37.03 | 9312 | 19896 | 0.47 |
| | 37.44 | 7816 | 16529 | 0.47 |
| HAdV F | 31.95 | 19007 | 22789 | 0.83 |
| 500 copies | 32.68 | 17294 | 21097 | 0.82 |
| | 32.27 | 18576 | 22010 | 0.84 |
| HAdV F | 36.39 | 9214 | 14897 | 0.62 |
| 50 copies | 35.88 | 8840 | 14549 | 0.61 |
| | 37.12 | 7808 | 15494 | 0.50 |

TABLE 10

| Adenovirus | Concentration | Average | |
|---|---|---|---|
| Subtype | (copies/rxn) | CT | SD |
| A | 500 | 35.22 | 0.55 |
| | 50 | 40.93 | 0.57 |
| B | 500 | 34.64 | 0.22 |
| | 50 | 39.26 | 0.57 |
| C | 500 | 32.12 | 0.15 |
| | 50 | 36.88 | 0.99 |
| D | 500 | 35.33 | 0.07 |
| | 50 | 39.02 | 1.02 |
| E | 500 | 35.12 | 0.48 |
| | 50 | 36.81 | 0.76 |
| F | 500 | 32.30 | 0.37 |
| | 50 | 36.46 | 0.62 |

TABLE 11

| | | Mean | Median - | Mean |
|---|---|---|---|---|
| Sample | CT | CT | CT | (Median - CT) |
| HAdV A | 35.82 | 35.22 | −1.11 | −0.50 |
| 500 copies | 35.11 | | −0.39 | |
| | 34.73 | | −0.01 | |
| HAdV B | 34.88 | 34.64 | −0.16 | 0.07 |
| 500 copies | 34.61 | | 0.11 | |
| | 34.44 | | 0.28 | |
| HAdV C | 31.96 | 32.12 | 2.76 | 2.60 |
| 500 copies | 32.25 | | 2.47 | |
| | 32.15 | | 2.57 | |
| HAdV D | 35.25 | 35.33 | −0.53 | −0.61 |
| 500 copies | 35.38 | | −0.66 | |
| | 35.36 | | −0.64 | |
| HAdV E | 35.01 | 35.12 | −0.29 | −0.40 |
| 500 copies | 34.70 | | 0.02 | |
| | 35.64 | | −0.92 | |
| HAdV F | 31.95 | 32.30 | 2.77 | 2.42 |
| 500 copies | 32.68 | | 2.04 | |
| | 32.27 | | 2.45 | |
| Median | 34.72 | | | |

TABLE 12

| | | | Median - | Mean |
|---|---|---|---|---|
| Sample | CT | Mean CT | CT | (Median - CT) |
| HAdV A | 40.80 | 40.93 | −2.95 | −3.08 |
| 50 copies | 40.44 | | −2.59 | |
| | 41.55 | | −3.70 | |
| HAdV B | 39.73 | 39.26 | −1.88 | −1.41 |
| 50 copies | 38.62 | | −0.77 | |
| | 39.42 | | −1.57 | |
| HAdV C | 35.76 | 36.88 | 2.09 | 0.97 |
| 50 copies | 37.23 | | 0.62 | |
| | 37.64 | | 0.21 | |
| HAdV D | 40.09 | 39.02 | −2.24 | −1.17 |
| 50 copies | 38.90 | | −1.05 | |
| | 38.06 | | −0.21 | |
| HAdV E | 35.96 | 36.81 | 1.89 | 1.04 |
| 50 copies | 37.03 | | 0.82 | |
| | 37.44 | | 0.41 | |
| HAdV F | 36.39 | 36.46 | 1.46 | 1.39 |
| 50 copies | 35.88 | | 1.97 | |
| | 37.12 | | 0.73 | |
| Median | 37.85 | | | |

The data demonstrate that the LoD of the HAdV plasmids for all six subtypes were at least 50 copies/reaction and confirms that this minimum LoD is still met in the presence of HeLa cells. Subtypes C, E, and F may actually be more sensitive based on the CTs observed. The data also show that subtypes C and F at 500 copies are currently out of the quantifiable range, as is subtype A at 50 copies; this will need to be optimized further (see Tables 11 and 12; the mean CT for each adenovirus subtype should be no more than 2.32 CTs from the median CT to be considered quantifiable).

### Example 6

This example describes the detection of adenovirus plasmid subtypes A-F with CalOrange 560-labeled probes.

1.25× primer and probe mixtures (PPR mixes) were prepared in a microfuge tube to include the oligonucleotides shown in Table 13 below. The internal control PPR mix comprised 0.75 μM of each primer and 0.5 μM of the probe, while the adenovirus PPR mixes contained 0.75 μM of each primer and 0.375 μM of each probe. These PPR mixes also contained 81.25 mM of KCl, 5 mM MgCl₂, and were brought to final volume in water.

For each of human adenovirus serotypes 18, 11, 5, 8, 4, and 41 (representing subtypes A, B, C, D, E, and F, respectively), HAdV plasmid stock was diluted in STM (supplemented with 10,000 HeLa cells/mL) to provide HAdV plasmids at 50, 500, 5,000, 50,000, 500,000, or 5,000,000 copies/reaction.

Amplification and detection reactions were set up at 6 reactions per condition; 3 reactions positive for the human adenovirus subtype and 3 reactions negative for the subtype. Negative reactions included sample transport media without adenovirus. The reactions were performed as real-time PCR reactions using a Panther Fusion system (available from Hologic, Inc., Marlborough MA). Results are shown in Table 14 below.

TABLE 13

| | SEQ ID | |
|---|---|---|
| Oligo | NO | Sequence (5′ to 3′) |
| HAdV Penton FP 1 | 3 | ACCGTCAGTGAAAACGT |
| HAdV Penton FP 2 | 48 | ACCGTTAGTGAAAACGT |
| HAdV Penton FP 3 | 49 | ACCATCAGTGAAAACGT |
| HAdV Penton RP 1 | 50 | GCCTTGTAGACGTAGGG |
| HAdV Penton RP 2 | 51 | GCCTTGTAAACGTAGGG |
| HAdV Penton RP 3 | 52 | GCTTTGTACACGTAAGG |
| HAdV Penton RP 4 | 9 | GCTTTGTGGACGTAGGG |
| HAdV Penton | 53 | Cal Fluor Orange 560- |
| PR 1_CalO | | CCTGCTCTCACAGATCACGG |
| | | GAC-BHQ1 |
| HAdV Penton | 16 | Cal Fluor Orange 560- |
| PR 2 CalO | | CCCGCCCTCACAGATCACG |
| | | GAAC-BHQ1 |
| HAdV Penton | 17 | Cal Fluor Orange 560- |
| PR 3 CalO | | CCCGCTCTCACAGATCACGG |
| | | GAC-BHQ1 |

Note:
Underlined C = 5-methyl cytosine

TABLE 14

| | | | | | | | | Average | SD |
|---|---|---|---|---|---|---|---|---|---|
| HAdV | Conc | Average | SD | Average | SD | Average | SD | Signal: | Signal: |
| Subtype | (copies/rxn) | CT | CT | RFU | RFU | Bkgd | Bkgd | Noise | Noise |
| A | 5.00E+06 | 18.26 | 0.06 | 34189.67 | 1552.78 | 1713.00 | 125.65 | 19.99 | 0.64 |
| | 5.00E+05 | 21.71 | 0.30 | 31513.33 | 2248.51 | 2239.33 | 166.56 | 14.07 | 0.11 |
| | 5.00E+04 | 24.75 | 0.07 | 28578.67 | 87.89 | 2455.33 | 55.30 | 11.64 | 0.25 |
| | 5.00E+03 | 28.91 | 0.32 | 19273.67 | 751.14 | 2120.33 | 117.37 | 9.11 | 0.57 |
| | 5.00E+02 | 32.98 | 0.07 | 12286.67 | 820.33 | 2220.33 | 51.63 | 5.53 | 0.26 |
| | 5.00E+01 | 37.21 | 0.66 | 6348.67 | 981.86 | 2242.67 | 244.11 | 2.83 | 0.32 |
| B | 5.00E+06 | 16.37 | 0.13 | 23275.67 | 1473.41 | 1663.00 | 94.30 | 14.00 | 0.46 |
| | 5.00E+05 | 19.78 | 0.08 | 25852.00 | 1885.64 | 2085.00 | 241.16 | 12.44 | 0.55 |
| | 5.00E+04 | 23.38 | 0.11 | 20836.33 | 2045.99 | 1896.00 | 129.95 | 10.98 | 0.32 |
| | 5.00E+03 | 27.01 | 0.15 | 20697.00 | 1976.70 | 2315.67 | 228.22 | 8.94 | 0.04 |
| | 5.00E+02 | 30.62 | 0.14 | 16687.00 | 565.85 | 2240.33 | 94.87 | 7.46 | 0.35 |
| | 5.00E+01 | 35.15 | 0.80 | 9688.33 | 1659.11 | 2315.67 | 276.43 | 4.17 | 0.23 |
| C | 5.00E+06 | 16.17 | 0.12 | 24573.33 | 1521.85 | 1857.67 | 143.31 | 13.24 | 0.20 |
| | 5.00E+05 | 19.59 | 0.04 | 26942.67 | 642.88 | 2373.00 | 54.67 | 11.35 | 0.07 |
| | 5.00E+04 | 23.21 | 0.25 | 25447.67 | 3057.57 | 2387.00 | 247.93 | 10.65 | 0.19 |
| | 5.00E+03 | 26.95 | 0.20 | 19244.00 | 1806.01 | 2145.00 | 170.99 | 8.96 | 0.15 |
| | 5.00E+02 | 30.36 | 0.21 | 17211.33 | 1975.95 | 2392.67 | 277.71 | 7.19 | 0.03 |
| | 5.00E+01 | 34.46 | 0.50 | 10931.00 | 1891.42 | 2178.00 | 180.27 | 5.00 | 0.46 |
| D | 5.00E+06 | 17.37 | 0.17 | 22908.67 | 1545.48 | 1978.33 | 165.56 | 11.59 | 0.27 |
| | 5.00E+05 | 20.82 | 0.06 | 21994.33 | 983.74 | 2059.00 | 153.53 | 10.70 | 0.48 |
| | 5.00E+04 | 24.17 | 0.14 | 21685.67 | 1528.14 | 2378.33 | 205.99 | 9.13 | 0.15 |
| | 5.00E+03 | 27.74 | 0.11 | 18577.67 | 605.76 | 2374.00 | 113.70 | 7.83 | 0.15 |
| | 5.00E+02 | 32.38 | 0.18 | 11534.33 | 1714.58 | 1994.33 | 259.90 | 5.78 | 0.29 |
| | 5.00E+01 | 37.17 | 0.76 | 5512.00 | 895.65 | 2157.33 | 120.79 | 2.55 | 0.38 |
| E | 5.00E+06 | 16.75 | 0.23 | 22931.33 | 2436.56 | 1893.33 | 243.75 | 12.13 | 0.33 |
| | 5.00E+05 | 20.00 | 0.08 | 24558.00 | 1482.98 | 2193.00 | 114.25 | 11.20 | 0.12 |
| | 5.00E+04 | 23.71 | 0.07 | 20394.33 | 710.04 | 1895.00 | 50.59 | 10.76 | 0.34 |
| | 5.00E+03 | 27.28 | 0.10 | 20837.33 | 1815.31 | 2337.67 | 215.18 | 8.92 | 0.25 |
| | 5.00E+02 | 30.88 | 0.07 | 16327.67 | 615.34 | 2276.33 | 24.17 | 7.17 | 0.22 |
| | 5.00E+01 | 35.59 | 0.56 | 9641.33 | 1651.92 | 2348.00 | 360.27 | 4.10 | 0.23 |
| F | 5.00E+06 | 15.43 | 0.14 | 33297.00 | 1916.84 | 2052.33 | 168.03 | 16.25 | 0.63 |
| | 5.00E+05 | 19.11 | 0.06 | 29081.67 | 2180.41 | 1864.67 | 111.63 | 15.59 | 0.23 |
| | 5.00E+04 | 22.51 | 0.04 | 29298.67 | 332.52 | 2259.33 | 93.54 | 12.98 | 0.41 |
| | 5.00E+03 | 26.02 | 0.28 | 26467.67 | 3021.00 | 2369.67 | 205.87 | 11.16 | 0.56 |
| | 5.00E+02 | 29.99 | 0.16 | 18158.67 | 2018.96 | 2101.67 | 201.87 | 8.63 | 0.22 |
| | 5.00E+01 | 34.66 | 0.20 | 10877.67 | 346.52 | 2399.33 | 233.29 | 4.56 | 0.47 |

Linearity was observed with all adenovirus subtypes. All titrated concentrations for all subtypes were detected and were also found to be within the 2.32 CT range of quantification. Background was low, resulting in higher signal-to-noise ratio calculations.

### Example 7

The performance of the primers and probes shown in Table 13 above in amplification and detection of human adenovirus was evaluated under higher and lower concentrations of probes, primers and MgCl₂ using HAdV plasmids for serotypes 18, 11, 5, 8, 4, and 41 (from HAdV subtypes A-F, respectively). Avg Ct, Avg RFU, and Avg slope at threshold were compared to control conditions (0.3 μM Probes, 0.6 μM Primers and 4 mM MgCl₂). The assay was run as a real-time PCR reaction on the Panther Fusion system. PCR formulations were prepared including all primers and probes listed in Table 13. Two extractions and six PCR replicates for each condition were performed. The samples were processed using the LDT-DNA-10-S version 2 protocol and the Wave 1 sequence file in which 360 μL of sample is extracted. 5 μL of eluate was used in the final PCR reaction. Plasmids were spiked into STM supplemented with HeLa cells for all tested conditions. The final concentration was 1000 copies/mL of the indicated plasmid and 10,000 HeLa cells/mL in STM.

Analysis of the results and associated amplification curves revealed that a probe concentration of 0.2 μM improved detection sensitivity over that of control conditions for all subtypes by >1 Ct. The analysis is summarized in Table 15 below. Detection of the A subtype using this probe concentration was also found to be more consistent with the other subtypes, improving quantification of the assay (median Ct difference between groups <2.3 Ct). Subsequent Examples herein use 0.2 μM probes, 0.6 μM primers, and 4 mM MgCl₂.

TABLE 15

| | HAdV A sero 18 | | HadV B sero 11 | | HadV C sero 5 | | HadV D sero 8 | | HadV E sero 4 | | HadV F sero 41 | | |
|---|---|---|---|---|---|---|---|---|---|---|---|---|---|
| | | Delta | | Delta | | Delta | | Delta | | Delta | | Delta | |
| | | Ct from | | Ct from | | Ct from | | Ct from | | Ct from | | Ct from | Median |
| | Ct | median | Ct | median | Ct | median | Ct | median | Ct | median | Ct | median | Ct |
| 0.2 μM | 30.53 | 1.96 | 28.55 | −0.03 | 28.31 | −0.27 | 29.70 | 1.13 | 28.60 | 0.03 | 27.39 | −1.19 | 28.58 |
| Probes | | | | | | | | | | | | | |
| 0.4 μM | 33.16 | 3.11 | 29.96 | −0.09 | 29.94 | −0.12 | 31.29 | 1.23 | 30.14 | 0.09 | 28.61 | −1.45 | 30.05 |
| Probes | | | | | | | | | | | | | |
| 0.5 μM | 31.62 | 1.78 | 29.70 | −0.14 | 29.37 | −0.48 | 31.28 | 1.44 | 29.99 | 0.14 | 28.74 | −1.11 | 29.85 |
| Primers | | | | | | | | | | | | | |
| 0.7 μM | 31.60 | 2.04 | 29.45 | −0.11 | 29.40 | −0.17 | 30.54 | 0.98 | 29.68 | 0.11 | 28.28 | −1.29 | 29.56 |
| Primers | | | | | | | | | | | | | |
| 3.5 mM | 34.61 | 4.37 | 30.26 | 0.02 | 30.07 | −0.17 | 32.08 | 1.84 | 30.22 | −0.02 | 28.78 | −1.46 | 30.24 |
| MgCl2 | | | | | | | | | | | | | |
| 4.5 mM | 31.70 | 2.37 | 29.18 | −0.14 | 29.22 | −0.10 | 30.41 | 1.08 | 29.43 | 0.10 | 28.20 | −1.13 | 29.32 |
| MgCl2 | | | | | | | | | | | | | |
| Control | 32.55 | 2.66 | 30.00 | 0.11 | 29.43 | −0.46 | 30.85 | 0.96 | 29.78 | −0.11 | 28.24 | −1.65 | 29.89 |

Control = 0.3 μM Probes, 0.6 μM Primers and 4 mM MgCl₂

### Example 8

60 HadV strains representing all serotypes that comprise subgroups A-F were evaluated for reactivity in STM supplemented with 10,000 HeLa cells/mL. The assay was run as a real-time PCR reaction on the Panther Fusion system using the primers and probes listed in Table 13. Two extractions and six PCR replicates for each condition were performed. The samples were processed using the LDT-DNA-10-S version 2 protocol and the Wave 1 sequence file in which 360 μL of sample is extracted. 5 μL of eluate was used in the final PCR reaction.

The analyte and the internal control were detected in all reactions. The data is summarized in Table 16.

TABLE 16

| | | | | | | | | | Avg | SD |
|---|---|---|---|---|---|---|---|---|---|---|
| | | | | | Avg | SD | Avg | SD | BKG | BKG |
| Subtype | Source | Serotype | TCID₅₀/mL | Reactivity | Ct | Ct | RFU | RFU | RFU | RFU |
| C | Zeptometrix | 1 | 2.50E+03 | 6/6 | 28.79 | 0.28 | 58747.83 | 2156.44 | 1998.33 | 322.67 |
| C | ATCC | 2 | 2.50E+03 | 6/6 | 20.00 | 0.26 | 89365.50 | 1419.36 | 1919.33 | 232.65 |
| B | Zeptometrix | 3 | 2.50E+03 | 6/6 | 24.76 | 0.15 | 76778.00 | 1974.84 | 1820.00 | 107.94 |
| E | Tricore | 4 | 2.50E+03 | 6/6 | 27.26 | 0.26 | 67521.00 | 2244.59 | 1823.33 | 220.16 |
| E | ATCC | 4a | 2.50E+03 | 6/6 | 26.66 | 0.45 | 64999.83 | 3483.83 | 2008.17 | 187.32 |
| E | Tricore | 4p3 | 2.50E+04 | 6/6 | 22.63 | 0.22 | 81730.17 | 1373.39 | 1740.00 | 216.84 |
| C | ATCC | 5 | 2.50E+04 | 6/6 | 24.53 | 0.20 | 81357.83 | 3349.02 | 1926.50 | 300.39 |
| C | Zeptometrix | 6 | 2.50E+03 | 6/6 | 19.35 | 0.15 | 93998.50 | 1051.44 | 2008.33 | 228.29 |
| B | ATCC | 7 | 2.50E+04 | 6/6 | 15.03 | 0.10 | 109989.83 | 7238.79 | 1588.17 | 322.68 |
| B | ATCC | 7a | 2.50E+03 | 6/6 | 25.25 | 0.12 | 78997.50 | 2966.31 | 1980.17 | 278.75 |
| B | Zeptometrix | 7d4 | 25000 | 6/6 | 19.33 | 0.30 | 94794.17 | 2486.46 | 1719.17 | 316.03 |
| B | ATCC | 7h1 | 2.50E+04 | 6/6 | 19.22 | 0.18 | 95735.17 | 1876.29 | 1755.17 | 236.03 |
| D | ATCC | 8 | 2.50E+04 | 6/6 | 13.36 | 0.42 | 101345.00 | 5661.95 | 1923.33 | 280.10 |
| D | Tricore | 9 | 2.50E+03 | 6/6 | 22.87 | 0.68 | 88559.67 | 2270.03 | 1814.50 | 185.92 |
| D | Tricore | 10 | 2.50E+03 | 6/6 | 26.44 | 0.30 | 74914.00 | 2368.96 | 2003.50 | 285.65 |
| B | ATCC | 11 | 2.50E+03 | 6/6 | 16.70 | 0.31 | 103785.83 | 2947.69 | 1847.00 | 262.15 |
| A | ATCC | 12 | 2.50E+03 | 6/6 | 27.93 | 0.32 | 34951.83 | 2721.50 | 2008.50 | 183.83 |
| A | Tricore | 12 | 2.50E+03 | 6/6 | 20.95 | 0.35 | 61744.50 | 9423.43 | 1550.33 | 230.20 |
| D | ATCC | 13 | 2.50E+04 | 6/6 | 19.27 | 0.31 | 95523.17 | 3368.30 | 1820.17 | 178.12 |
| B | Tricore | 14 | 2.50E+04 | 6/6 | 15.89 | 0.10 | 102834.17 | 4726.55 | 1717.00 | 261.23 |
| B | ATCC | 14-4 | 2.50E+04 | 6/6 | 18.70 | 0.15 | 104413.83 | 1648.44 | 1683.17 | 132.29 |
| D | ATCC | 15 | 2.50E+04 | 6/6 | 18.20 | 0.44 | 100137.67 | 3348.20 | 1498.17 | 184.60 |
| B | ATCC | 16 | 2.50E+04 | 6/6 | 18.29 | 0.50 | 98027.50 | 6796.07 | 1733.67 | 229.85 |
| D | Tricore | 17 | 2.50E+03 | 6/6 | 22.89 | 0.40 | 88432.00 | 671.21 | 1983.33 | 141.53 |
| A | ATCC | 18 | 2.50E+03 | 6/6 | 25.76 | 0.30 | 89225.67 | 2183.93 | 1738.17 | 320.50 |
| D | ATCC | 19 | 2.50E+03 | 6/6 | 20.03 | 0.45 | 97435.00 | 5486.69 | 1722.83 | 168.02 |
| D | Tricore | 20 | 2.50E+03 | 6/6 | 19.57 | 0.15 | 93545.00 | 1943.88 | 1881.33 | 236.18 |
| B | Tricore | 21 | 2.50E+04 | 6/6 | 18.15 | 0.25 | 101619.00 | 4911.25 | 1785.00 | 179.17 |
| B | Zeptometrix | 21a | 2.50E+04 | 6/6 | 20.29 | 0.04 | 89761.83 | 1982.37 | 1797.33 | 350.02 |
| D | ATCC | 22 | 2.50E+04 | 6/6 | 14.72 | 0.24 | 102927.67 | 4755.73 | 1688.67 | 256.75 |
| D | Tricore | 23 | 2.50E+04 | 6/6 | 13.87 | 0.31 | 102399.83 | 4135.16 | 1918.83 | 190.78 |
| D | Tricore | 24 | 2.50E+04 | 6/6 | 13.31 | 0.19 | 103656.17 | 4828.03 | 1706.33 | 293.59 |
| D | ATCC | 25 | 2.50E+04 | 6/6 | 19.69 | 0.18 | 100156.00 | 4154.81 | 1719.00 | 159.98 |
| D | Zeptometrix | 26 | 2.50E+03 | 6/6 | 14.68 | 0.35 | 115479 | 1747.00 | 1642.00 | 139.00 |
| D | Tricore | 27 | 2.50E+03 | 6/6 | 14.59 | 0.19 | 97614.17 | 5614.10 | 1707.83 | 218.55 |
| D | Zeptometrix | 28 | 2.50E+03 | 6/6 | 23.92 | 0.63 | 83350.17 | 4031.91 | 2094.50 | 246.58 |
| D | ATCC | 29 | 2.50E+04 | 6/6 | 17.84 | 0.36 | 102855.50 | 2595.53 | 1624.00 | 258.04 |
| D | ATCC | 30 | 2.50E+04 | 6/6 | 15.28 | 0.31 | 108680.00 | 7344.62 | 1452.17 | 275.57 |
| A | Tricore | 31 | 2.50E+04 | 6/6 | 15.98 | 0.10 | 140851.00 | 10095.22 | 1640.83 | 238.61 |
| D | Zeptometrix | 32 | 2.50E+03 | 6/6 | 20.74 | 0.23 | 91109.00 | 2774.21 | 1795.83 | 259.65 |
| D | ATCC | 33 | 2.50E+03 | 6/6 | 21.62 | 0.31 | 93096.00 | 4749.16 | 1737.17 | 298.32 |
| B | Tricore | 34 | 2.50E+03 | 6/6 | 17.22 | 0.13 | 109270.67 | 3644.41 | 1711.50 | 216.66 |
| B | Zeptometrix | 35 | 2.50E+03 | 6/6 | 14.40 | 0.12 | 101277.00 | 7660.56 | 1904.83 | 294.65 |
| D | ATCC | 36 | 2.50E+03 | 6/6 | 17.92 | 0.29 | 98692.67 | 2530.85 | 1919.00 | 139.77 |
| D | ATCC | 37 | 2.50E+04 | 6/6 | 12.63 | 0.24 | 112555.00 | 7019.94 | 1558.50 | 200.83 |
| D | Tricore | 38 | 2.50E+04 | 6/6 | 15.37 | 0.27 | 113325.83 | 4936.32 | 1692.17 | 225.02 |
| D | Zeptometrix | 39 | 2.50E+04 | 6/6 | 17.53 | 0.32 | 104745.17 | 8594.60 | 1607.67 | 283.13 |
| F | ATCC | 40 | 2.50E+04 | 6/6 | 12.12 | 0.23 | 143842.67 | 5377.17 | 1747.00 | 141.34 |
| F | Tricore | 41 | 2.50E+03 | 6/6 | 16.00 | 0.17 | 175083.67 | 6860.69 | 1479.83 | 179.00 |
| D | Tricore | 42 | 2.50E+03 | 6/6 | 12.44 | 0.02 | 101975.67 | 3540.21 | 1705.83 | 314.52 |
| D | Tricore | 43 | 2.50E+03 | 6/6 | 16.62 | 0.09 | 101172.17 | 1597.91 | 1797.17 | 226.88 |
| D | ATCC | 44 | 2.50E+03 | 6/6 | 14.99 | 0.30 | 111918.33 | 4119.98 | 1610.00 | 271.18 |
| D | Tricore | 45 | 2.50E+04 | 6/6 | 17.19 | 0.17 | 103027.00 | 2026.27 | 1696.50 | 242.50 |
| D | Zeptometrix | 46 | 2.50E+04 | 6/6 | 18.14 | 1.00 | 104482.67 | 3170.44 | 1583.33 | 153.94 |
| D | ATCC | 47 | 2.50E+04 | 6/6 | 12.15 | 0.07 | 109291.83 | 4202.98 | 1744.67 | 236.60 |
| D | ATCC | 48 | 2.50E+04 | 6/6 | 18.09 | 0.88 | 95397.17 | 2433.64 | 1805.67 | 97.49 |
| D | Tricore | 49 | 2.50E+03 | 6/6 | 21.24 | 0.35 | 96551.17 | 2064.72 | 1565.50 | 193.48 |
| B | ATCC | 50 | 2.50E+03 | 6/6 | 20.35 | 0.08 | 94071.83 | 1897.54 | 1964.67 | 233.68 |
| D | Zeptometrix | 21c | 2.50E+03 | 6/6 | 24.74 | 0.21 | 79531.83 | 2427.77 | 1912.50 | 324.48 |
| D | ATCC | 51 | 2.50E+03 | 6/6 | 26.57 | 0.63 | 90062.17 | 4248.13 | 1774.00 | 182.79 |

### Example 9: Plasmid Sensitivity and Lower Limit of Detection (LLOD)

Plasmid sensitivity was evaluated by testing HAdV plasmids representative of each subtype in STM supplemented with 10,000 HeLa cells/mL at seven concentrations between 5-5,000,000 copies/reaction, as listed in Table 17. The assay was run as a real-time PCR reaction on the Panther Fusion system using the primers and probes listed in Table 13. Plasmids were spiked into STM at the indicated concentrations. The plasmid spiked samples containing HeLa cells were lysed, and the plasmids were purified from these lysates. 5 μL of eluate was used in the final PCR reaction. The data is summarized in Table 17 below. 100% detection of HAdV was seen down to 50 cp/rxn for subtypes A to F. PCR efficiency was high with R² values >0.99 and slopes at −3.5, −3.6, −3.6, −3.5, −3.6, −3.6, −3.6 for serotypes 12, 18, 11, 5, 8, 4, and 41, respectively. The internal control exhibited 100% detection in all dilutions for all serotypes.

TABLE 17
Plasmid Sensitivity Results

| | | Conc. | | Avg | StdDev | Avg | StdDev | Avg T- | StdDev |
|---|---|---|---|---|---|---|---|---|---|
| Subtype | Serotype | (cp/rxn) | Reactivity | Ct | Ct | RFU | RFU | Slope | T-Slope |
| A | 12 | 5,000,000 | 3/3 | 18.96 | 0.14 | 80165.33 | 3811.97 | 1046.67 | 183.33 |
| | | 500,000 | 3/3 | 22.42 | 0.08 | 63242.00 | 3102.59 | 971.00 | 52.12 |
| | | 50,000 | 3/3 | 25.72 | 0.10 | 50892.33 | 2114.12 | 853.00 | 49.51 |
| | | 5,000 | 3/3 | 29.04 | 0.11 | 38426.00 | 2704.00 | 1005.00 | 189.93 |
| | | 500 | 3/3 | 33.15 | 0.26 | 22556.67 | 2612.66 | 794.67 | 92.46 |
| | | 50 | 3/3 | 37.25 | 0.81 | 10593.67 | 2171.03 | 659.67 | 45.35 |
| | | 5 | 3/3 | 41.95 | 0.73 | 3668.00 | 941.74 | 721.67 | 42.91 |
| A | 18 | 5,000,000 | 6/6 | 16.54 | 0.18 | 224320.00 | 10979.61 | 1031.33 | 114.30 |
| | | 500,000 | 6/6 | 20.19 | 0.16 | 175855.00 | 4302.64 | 1256.50 | 125.66 |
| | | 50,000 | 6/6 | 23.28 | 0.11 | 145227.17 | 4038.20 | 1180.67 | 74.42 |
| | | 5,000 | 6/6 | 27.26 | 0.23 | 105368.83 | 4170.12 | 1214.17 | 180.34 |
| | | 500 | 6/6 | 30.91 | 0.25 | 75059.67 | 4796.62 | 942.00 | 132.16 |
| | | 50 | 6/6 | 34.64 | 0.48 | 42552.17 | 6034.52 | 993.83 | 186.21 |
| | | 5 | 5/6 | 39.11 | 1.26 | 11650.67 | 8432.67 | 947.80 | 135.34 |
| B | 11 | 5,000,000 | 6/6 | 17.06 | 0.08 | 135164.83 | 3068.96 | 1196.67 | 280.69 |
| | | 500,000 | 6/6 | 20.51 | 0.06 | 123705.33 | 4508.83 | 1067.67 | 35.09 |
| | | 50,000 | 6/6 | 24.11 | 0.06 | 108955.00 | 3192.32 | 1327.00 | 59.85 |
| | | 5,000 | 6/6 | 27.50 | 0.12 | 92976.67 | 3821.53 | 1052.33 | 83.57 |
| | | 500 | 6/6 | 31.20 | 0.12 | 68871.17 | 2442.28 | 1135.00 | 54.78 |
| | | 50 | 6/6 | 35.18 | 0.30 | 39617.50 | 2934.96 | 901.00 | 144.80 |
| | | 5 | 6/6 | 39.14 | 0.86 | 14582.33 | 4224.51 | 966.00 | 107.16 |
| C | 5 | 5,000,000 | 6/6 | 16.41 | 0.15 | 142115.33 | 5084.50 | 1163.50 | 102.25 |
| | | 500,000 | 6/6 | 19.87 | 0.15 | 126195.50 | 3111.05 | 1085.33 | 259.79 |
| | | 50,000 | 6/6 | 23.32 | 0.04 | 112128.33 | 2976.73 | 1152.33 | 33.42 |
| | | 5,000 | 6/6 | 26.81 | 0.11 | 95340.67 | 999.32 | 888.00 | 36.60 |
| | | 500 | 6/6 | 30.46 | 0.24 | 72462.67 | 3026.14 | 1026.17 | 134.40 |
| | | 50 | 6/6 | 34.11 | 0.27 | 48995.50 | 5651.49 | 880.50 | 134.92 |
| | | 5 | 5/6 | 39.83 | 1.27 | 9738.00 | 6745.33 | 821.40 | 140.71 |
| D | 8 | 5,000,000 | 6/6 | 17.23 | 0.10 | 131214.83 | 2367.98 | 1295.17 | 91.82 |
| | | 500,000 | 6/6 | 20.86 | 0.09 | 117607.67 | 4053.46 | 876.00 | 40.78 |
| | | 50,000 | 6/6 | 24.40 | 0.14 | 103229.50 | 1769.92 | 1101.33 | 86.43 |
| | | 5,000 | 6/6 | 27.72 | 0.15 | 86245.33 | 3281.46 | 925.50 | 56.63 |
| | | 500 | 6/6 | 31.62 | 0.14 | 61904.00 | 1957.06 | 876.83 | 48.34 |
| | | 50 | 6/6 | 35.63 | 0.62 | 36572.67 | 4337.44 | 1009.17 | 142.65 |
| | | 5 | 6/6 | 40.07 | 1.49 | 11355.00 | 7196.92 | 941.83 | 129.60 |
| E | 4 | 5,000,000 | 6/6 | 16.35 | 0.14 | 142891.33 | 7869.61 | 1210.50 | 137.93 |
| | | 500,000 | 6/6 | 20.08 | 0.10 | 121561.67 | 2339.69 | 1198.83 | 267.05 |
| | | 50,000 | 6/6 | 23.62 | 0.15 | 113837.00 | 2787.34 | 1007.00 | 86.93 |
| | | 5,000 | 6/6 | 27.13 | 0.05 | 97371.00 | 3998.45 | 1326.67 | 52.44 |
| | | 500 | 6/6 | 30.67 | 0.19 | 76271.17 | 1889.83 | 924.17 | 76.38 |
| | | 50 | 6/6 | 34.69 | 0.50 | 44917.17 | 5143.70 | 982.33 | 129.21 |
| | | 5 | 4/6 | 38.44 | 1.08 | 10560.50 | 9169.18 | 874.50 | 129.79 |
| F | 41 | 5,000,000 | 6/6 | 17.02 | 0.13 | 203222.83 | 10125.33 | 1246.50 | 269.23 |
| | | 500,000 | 6/6 | 20.27 | 0.13 | 175873.00 | 7055.45 | 1243.17 | 114.68 |
| | | 50,000 | 6/6 | 23.88 | 0.17 | 149971.17 | 4007.39 | 976.17 | 210.86 |
| | | 5,000 | 6/6 | 27.36 | 0.09 | 112766.50 | 5049.99 | 1154.83 | 63.93 |
| | | 500 | 6/6 | 31.15 | 0.15 | 76868.83 | 5539.41 | 1030.00 | 172.66 |
| | | 50 | 5/5 | 35.63 | 0.75 | 30022.17 | 15786.13 | 919.40 | 148.77 |
| | | 5 | 4/6 | 38.49 | 0.70 | 11958.50 | 10095.94 | 795.50 | 66.38 |

*Reactivity defined as an amplification curve crossing a threshold of 1700 RFU.

The median Ct values for all serotypes are summarized in Table 18 below. All the tested serotypes are within 2.3 Ct from the median of the Ct values, demonstrating that all tested serotypes can be quantified with one set of standard curves.

TABLE 18
Plasmid Sensitivity: Median Ct Values

| Subtype | | | | Delta Ct from |
|---|---|---|---|---|
| (Serotype) | cp/rxn | Ct Avg | Median | Median |
| HAdV A (12) | 50 | 37.25 | 35.18 | 2.07 |
| HAdV A (18) | | 34.64 | | −0.54 |
| HAdV B (11) | | 35.18 | | 0.00 |
| HAdV C (5) | | 34.11 | | −1.07 |
| HAdV D (8) | | 35.63 | | 0.45 |
| HAdV E (4) | | 34.69 | | −0.49 |
| HAdV F (41) | | 35.63 | | 0.46 |
| HAdV A (12) | 500 | 33.15 | 31.15 | 2.00 |
| HAdV A (18) | | 30.91 | | −0.24 |
| HAdV B (11) | | 31.20 | | 0.05 |
| HAdV C (5) | | 30.46 | | −0.68 |
| HAdV D (8) | | 31.62 | | 0.48 |
| HAdV E (4) | | 30.67 | | −0.48 |
| HAdV F (41) | | 31.15 | | 0.00 |
| HAdV A (12) | 5,000 | 29.04 | 27.36 | 1.69 |
| HAdV A (18) | | 27.26 | | −0.10 |
| HAdV B (11) | | 27.50 | | 0.14 |
| HAdV C (5) | | 26.81 | | −0.55 |
| HAdV D (8) | | 27.72 | | 0.36 |
| HAdV E (4) | | 27.13 | | −0.23 |
| HAdV F (41) | | 27.36 | | 0.00 |
| HAdV A (12) | 50,000 | 25.72 | 23.88 | 1.85 |
| HAdV A (18) | | 23.28 | | −0.60 |
| HAdV B (11) | | 24.11 | | 0.23 |
| HAdV C (5) | | 23.32 | | −0.56 |
| HAdV D (8) | | 24.40 | | 0.52 |
| HAdV E (4) | | 23.62 | | −0.26 |
| HAdV F (41) | | 23.88 | | 0.00 |
| HAdV A (12) | 500,000 | 22.42 | 20.27 | 2.15 |
| HAdV A (18) | | 20.19 | | −0.08 |
| HAdV B (11) | | 20.51 | | 0.24 |
| HAdV C (5) | | 19.87 | | −0.40 |
| HAdV D (8) | | 20.86 | | 0.59 |
| HAdV E (4) | | 20.08 | | −0.19 |
| HAdV F (41) | | 20.27 | | 0.00 |
| HAdV A (12) | 5,000,000 | 18.96 | 17.02 | 1.94 |
| HAdV A (18) | | 16.54 | | −0.48 |
| HAdV B (11) | | 17.06 | | 0.04 |
| HAdV C (5) | | 16.41 | | −0.61 |
| HAdV D (8) | | 17.23 | | 0.21 |
| HAdV E (4) | | 16.35 | | −0.67 |
| HAdV F (41) | | 17.02 | | 0.00 |

### Example 10: Viral Sensitivity and LLOD

Eight HAdV strains were tested for sensitivity with a PCR formulation including all primers and probes listed in Table 13. A summary of tested samples and their associated matrices are shown in Table 19 below. The assay was run as a real-time PCR reaction using the Panther Fusion system. Virus was spiked into either STM containing 10,000 HeLa cells/mL, or pooled plasma in a 10-fold dilution series. Eight concentrations were used ranging from 2.5E3 TCID50/mL to 2.5E-4 TCID50/mL. The samples in STM and pooled plasma samples were processed and amplification and detection was performed as generally described above.

Results for strains in STM are shown in Table 20 below. 100% detection (6/6 replicates) was observed for viral strains down to a concentration of 2.5 TCID50/mL with the exception of HAdV B serotype 3, which showed 83.3% detection (5/6 replicates) at this concentration.

Serotypes 4 and 7 were chosen for evaluation within pooled plasma. Both serotypes are relatively common, and serotype 7 is associated with severe clinical outcomes according to the CDC. Results for serotypes 4 and 7 are shown in Table 21 below. Pooled plasma alone was used as a negative control to confirm the absence of HAdV. 100% detection was observed down to 2.5 TCID50/mL for these two strains. A summary of virus linearity and LOQ for viral strains in STM and pooled plasma is shown in Table 22 below.

TABLE 19
Viral Sensitivity and LLOD Samples

| Sub- | | | |
|---|---|---|---|
| type | Serotype | Source | Matrix |
| A | 12 | ATCC | HeLa cells in STM at 10,000 cells/mL |
| A | 12 | Tricore | |
| B | 3 | Zeptometrix | |
| B | 7 | Tricore | Pooled plasma |
| C | 2 | ATCC | HeLa cells in STM at 10,000 cells/mL |
| D | 8 | Zeptometrix | |
| E | 4 | Tricore | Pooled plasma |
| F | 40 | Zeptometrix | HeLa cells in STM at 10,000 cells/mL |

TABLE 20
Virus Sensitivity Results: STM

| | | | | | Avg | SD | Avg | SD |
|---|---|---|---|---|---|---|---|---|
| Subtype | Source | Serotype | TCID₅₀/mL | Reactivity | Ct | Ct | RFU | RFU |
| A | Tricore | 12 | 2.50E+03 | 6/6 | 21.25 | 0.17 | 71648.67 | 5887.32 |
| | | | 2.50E+02 | 6/6 | 24.52 | 0.40 | 56268.00 | 2324.16 |
| | | | 2.50E+01 | 6/6 | 27.86 | 0.27 | 42845.17 | 3456.39 |
| | | | 2.50E+00 | 6/6 | 31.38 | 0.24 | 31527.17 | 2275.68 |
| | | | 2.50E−01 | 6/6 | 35.50 | 0.54 | 18543.83 | 2100.52 |
| | | | 2.50E−02 | 6/6 | 37.70 | 1.09 | 13930.83 | 4317.53 |
| | | | 2.50E−03 | 0/6 | 0.00 | 0.00 | 87.17 | 221.14 |
| | | | 2.50E−04 | 0/6 | 0.00 | 0.00 | 63.50 | 137.08 |
| A | ATCC | 12 | 2.50E+03 | 6/6 | 27.50 | 0.11 | 43352.00 | 3877.30 |
| | | | 2.50E+02 | 6/6 | 30.95 | 0.30 | 31796.17 | 2776.51 |
| | | | 2.50E+01 | 6/6 | 35.12 | 0.32 | 20763.50 | 2909.23 |
| | | | 2.50E+00 | 6/6 | 39.01 | 1.06 | 11841.17 | 7682.46 |
| | | | 2.50E−01 | 1/6 | 41.29 | 0.00 | 726.33 | 1783.60 |
| | | | 2.50E−02 | 0/6 | 0.00 | 0.00 | −1.17 | 12.29 |
| | | | 2.50E−03 | 1/6 | 39.22 | 0.00 | 2017.67 | 4934.48 |
| | | | 2.50E−04 | 0/6 | 0.00 | 0.00 | 0.83 | 16.36 |
| B | Zeptometrix | 3 | 2.50E+03 | 6/6 | 25.56 | 0.42 | 97479.17 | 2758.77 |
| | | | 2.50E+02 | 6/6 | 29.03 | 0.33 | 78290.33 | 1945.26 |
| | | | 2.50E+01 | 6/6 | 33.08 | 0.69 | 51406.33 | 4945.54 |
| | | | 2.50E+00 | 5/6 | 37.43 | 1.41 | 18780.33 | 11033.59 |
| | | | 2.50E−01 | 6/6 | 32.70 | 0.35 | 56505.83 | 1858.89 |
| | | | 2.50E−02 | 5/6 | 37.07 | 0.63 | 19755.00 | 10187.13 |
| | | | 2.50E−03 | 6/6 | 39.05 | 2.42 | 16174.17 | 10001.90 |
| | | | 2.50E−04 | 6/6 | 37.12 | 1.57 | 24926.67 | 9508.94 |
| C | ATCC | 2 | 2.50E+03 | 6/6 | 22.59 | 0.23 | 113777.67 | 2103.50 |
| | | | 2.50E+02 | 6/6 | 25.57 | 0.16 | 104394.83 | 2308.05 |
| | | | 2.50E+01 | 6/6 | 28.45 | 0.31 | 89509.83 | 4356.48 |
| | | | 2.50E+00 | 6/6 | 32.80 | 0.29 | 57761.17 | 4535.30 |
| | | | 2.50E−01 | 6/6 | 36.69 | 0.52 | 31291.17 | 9460.56 |
| | | | 2.50E−02 | 5/6 | 39.60 | 1.79 | 11432.67 | 9757.12 |
| | | | 2.50E−03 | 6/6 | 38.59 | 1.27 | 17783.83 | 8495.97 |
| | | | 2.50E−04 | 4/6 | 38.81 | 1.41 | 11161.17 | 11660.39 |
| D | Zeptometrix | 8 | 2.50E+03 | 6/6 | 15.45 | 0.23 | 135353.83 | 6982.66 |
| | | | 2.50E+02 | 6/6 | 18.98 | 0.29 | 126424.83 | 2421.63 |
| | | | 2.50E+01 | 6/6 | 22.29 | 0.26 | 116409.50 | 4786.16 |
| | | | 2.50E+00 | 6/6 | 25.56 | 0.17 | 108928.00 | 3952.37 |
| | | | 2.50E−01 | 6/6 | 29.09 | 0.16 | 88216.33 | 2867.91 |
| | | | 2.50E−02 | 6/6 | 32.33 | 0.15 | 67498.00 | 5800.71 |
| | | | 2.50E−03 | 6/6 | 33.72 | 0.48 | 53072.67 | 5072.98 |
| | | | 2.50E−04 | 6/6 | 31.84 | 0.24 | 63790.50 | 4847.34 |
| F | Zeptometrix | 40 | 2.50E+03 | 6/6 | 18.50 | 0.19 | 172798.50 | 7648.43 |
| | | | 2.50E+02 | 6/6 | 22.10 | 0.42 | 148389.83 | 5243.25 |
| | | | 2.50E+01 | 6/6 | 25.54 | 0.32 | 126059.50 | 4687.56 |
| | | | 2.50E+00 | 6/6 | 29.11 | 0.18 | 96385.83 | 5346.10 |
| | | | 2.50E−01 | 6/6 | 31.73 | 0.31 | 98203.17 | 5605.49 |
| | | | 2.50E−02 | 6/6 | 33.13 | 0.29 | 74289.50 | 3782.91 |
| | | | 2.50E−03 | 6/6 | 35.13 | 0.30 | 51043.50 | 7157.39 |
| | | | 2.50E−04 | 6/6 | 38.43 | 0.61 | 18044.00 | 4095.72 |

TABLE 21
Virus Sensitivity Results: Pooled Plasma

| Subtype | Serotype | TCID₅₀/mL | Reactivity | Avg Ct | SD Ct | Avg RFU | SD RFU |
|---|---|---|---|---|---|---|---|
| B | 7 | 2.50E+03 | 6/6 | 24.40 | 0.19 | 94332.50 | 2545.75 |
| | | 2.50E+02 | 6/6 | 27.97 | 0.23 | 76856.83 | 1868.55 |
| | | 2.50E+01 | 6/6 | 31.93 | 0.26 | 55982.83 | 2921.51 |
| | | 2.50E+00 | 6/6 | 35.97 | 0.45 | 31499.67 | 3391.52 |
| | | 2.50E−01 | 5/6 | 40.21 | 1.33 | 8054.33 | 6001.99 |
| E | 4 | 2.50E+03 | 6/6 | 27.77 | 0.27 | 84501.00 | 1373.16 |
| | | 2.50E+02 | 6/6 | 31.97 | 0.25 | 61094.00 | 1913.43 |
| | | 2.50E+01 | 6/6 | 35.75 | 0.78 | 34559.33 | 4923.17 |
| | | 2.50E+00 | 6/6 | 39.87 | 1.00 | 11156.50 | 4423.13 |
| | | 2.50E−01 | 1/6 | 42.23 | 0.00 | 673.83 | 1509.61 |
| Plasma | 3 | N/A | 0/3 | 0.00 | 0.00 | −21.67 | 11.15 |
| neg Ctrl | | | | | | | |

TABLE 22
Summary of Virus Linearity

| Sub- | | | LOQ | | |
|---|---|---|---|---|---|
| type | Serotype | Matrix | (TCID₅₀/mL) | Slope | Intercept |
| A | 12(Tricore) | STM + HeLa | 0.25 | −3.5 | 30.9 |
| A | 12(ATCC) | STM + HeLa | 2.5 | −3.7 | 38.1 |
| B | 3 | STM + HeLa | 25 | −3.7 | 36 |
| C | 2 | STM + HeLa | 0.25 | −3.5 | 32 |
| D | 8 | STM + HeLa | 0.025 | −3.3 | 25.1 |
| F | 40 | STM + HeLa | 0.25 | −3.3 | 28.2 |
| B | 7 | Plasma | 0.25 | −3.9 | 37.4 |
| E | 4 | Plasma | 2.5 | −4 | 41.45 |

### Example 11: Specificity

HAdV reactivity was evaluated in 35 organisms commonly found in blood. 12 panels grouped by organism relationship were studied using a PCR formulation including all primers and probes listed in Table 13. The assay was run as a real-time PCR reaction using the Panther Fusion system. The samples were processed as described above and 5 μL of eluate was used in the final PCR reaction. 1 extraction and 3 PCR replicates were performed for each panel. Organism panels were spiked into STM and reactivity was tested in the absence of HAdV material. HAdV E subtype 4 was also spiked into STM at a concentration of 2.5E2 TCID50/mL as a positive control.

Panel composition and reactivity results are shown in Table 23 below. All organisms tested were negative for HAdV except for 1 replicate for panel 2. However, the negative control (no template) also showed weak amplification (Ct=39 for ⅓ replicates). Both Panel 2 and the negative control were retested and they were all negative for HAdV. The late Ct values in the first run for one rep of the negative control and one rep of panel 2 were likely caused by operator error.

TABLE 23
Specificity Results

| | | | | Panel | Final | |
|---|---|---|---|---|---|---|
| Panel | Organism | Strain | Units | Conc. (10×) | Conc.(1:10) | Reactivity |
| 1 | Varicella Zoster Virus (VZV) | Isolate A | cp/mL | 1.00E+07 | 1.00E+06 | 0/3 |
| | Epstein-Barr Virus (EBV) | B95-8 | cp/mL | 1.00E+07 | 1.00E+06 | |
| | BK Virus | N/A | cp/mL | 1.00E+07 | 1.00E+06 | |
| | Cytomegalovirus AD-169 | AD-169 | cp/mL | 1.00E+07 | 1.00E+06 | |
| | Cell culture | | | | | |
| 2 | Candida albicans | CBS 562 | CFU/mL | 1.00E+07 | 1.00E+06 | 1/3 in the |
| | Chlamydia trachomatis | BOUR | IFU/mL | 1.00E+07 | 1.00E+06 | 1^{st} run, 0/3 |
| | HIV-1 | B | cp/mL | 1.00E+07 | 1.00E+06 | in the 2^{nd} run |
| 3 | Dengue Virus Type 1 | Hawaii | TCID₅₀/mL | 5.00E+04 | 5.00E+03 | 0/3 |
| | Dengue Virus Type 2 | New Guinea | TCID₅₀/mL | 5.00E+04 | 5.00E+03 | |
| | | C | | | | |
| | Dengue Virus Type 3 | H87 | TCID₅₀/mL | 1.43E+06 | 1.43E+05 | |
| 4 | Herpes Simplex Virus Type | MS | TCID₅₀/mL | 1.43E+05 | 1.43E+04 | 0/3 |
| | 2 (HSV-2) | | | | | |
| | HIV Type 2 (HIV-2) | NIH-Z | TCID₅₀/mL | 1.00E+04 | 1.00E+03 | |
| | HPV type 18 purified | 18 | cp/mL | 1.00E+07 | 1.00E+06 | |
| | plasmid DNA | | | | | |
| 5 | Human Herpes Virus Type | GS | cp/mL | 1.00E+07 | 1.00E+06 | 0/3 |
| | 6A (HHV-6A) | | | | | |
| | Human Herpes Virus Type | Z29 | cp/mL | 1.00E+07 | 1.00E+06 | |
| | 6B (HHV-6B) | | | | | |
| | Human Herpes Virus Type 7 | SB | TCID₅₀/mL | 1.00E+06 | 1.00E+05 | |
| | (HHV-7) | | | | | |
| 6 | Human T-Lymphotropic | N/A | vp/mL | 1.00E+07 | 1.00E+06 | 0/3 |
| | Virus Type I (HTLV-I) | | | | | |
| | Human T-Lymphotropic | N/A | vp/mL | 1.00E+07 | 1.00E+06 | |
| | Virus Type II (HTLV-II) | | | | | |
| | Human Hepatitis B Virus | N/A | IU/mL | 1.79E+05 | 1.79E+04 | |
| | (HBV) | | | | | |
| 7 | Mycobacterium smegmatis | W-113 | CFU/mL | 1.00E+07 | 1.00E+06 | 0/3 |
| | Neisseria gonorrhoeae | NCTC 8375 | CFU/mL | 1.00E+07 | 1.00E+06 | |
| | Propionibacterium acnes | NCTC 737 | CFU/mL | 1.00E+07 | 1.00E+06 | |
| 8 | West Nile Virus (WNV) | NY 2001- | cp/mL | 1.00E+04 | 1.00E+03 | 0/3 |
| | | 6263 | | | | |
| | Vaccinia Virus Culture Fluid | “Vaccine | TCID₅₀/mL | 1.00E+07 | 1.00E+06 | |
| | | Strain” | | | | |
| | Trichomonas vaginalis | JH 31A #4 | cells/mL | 1.00E+06 | 1.00E+05 | |
| 9 | Staphylococcus epidermidis | RP62A | CFU/mL | 1.00E+07 | 1.00E+06 | 0/3 |
| | Human Parvovirus | B19 | IU/mL | 1.00E+07 | 1.00E+06 | |
| 10 | Hepatitis A virus | HM175 | IU/mL | 9.95E+06 | 9.95E+05 | 0/3 |
| | Dengue Virus Type 4 | H241 | TCID₅₀/mL | 5.00E+06 | 5.00E+05 | |
| | Synthetic Human | 16 | cp/mL | 1.00E+04 | 1.00E+03 | |
| | Papillomavirus 16 DNA | | | | | |
| 11 | Human Herpes Virus Type 8 | N/A | cp/mL | 1.00E+07 | 1.00E+06 | 0/3 |
| | (HHV-8) | | | | | |
| | Human Hepatitis C Virus | N/A | IU/mL | 2.63E+04 | 2.63E+03 | |
| | (HCV) | | | | | |
| | Staphylococcus aureus | NCTC 8532 | CFU/mL | 1.00E+07 | 1.00E+06 | |
| 12 | HSV-1 Strain | MacIntyre | TCID₅₀/mL | 1.00E+06 | 1.00E+05 | 0/3 |
| | Mycobacterium gordonae | L. Wayne | cp/mL | 1.00E+07 | 1.00E+06 | |
| | | W-1609 | | | | |

### Example 12: Interference

Interference was also tested simultaneously with the specificity experiment of Example 11. Reactivity for HAdV E serotype 4 at a concentration of 250 TCID50/ml was evaluated in the presence of the panels from the specificity experiment. Composition and reactivity results are shown in Table 24 below. HAdV was detected in 100% of the panels. The data is summarized in Table 25 below.

TABLE 24
HAdV Oligo Multiplex Performance in the Presence of Common Organisms

| | | | | Panel | Final | |
|---|---|---|---|---|---|---|
| Panel | Organism | Strain | Units | Conc. (10×) | Conc.(1:10) | Reactivity |
| 1 | Varicella Zoster Virus (VZV) | Isolate A | cp/mL | 1.00E+07 | 1.00E+06 | 3/3 |
| | Epstein-Barr Virus (EBV) | B95-8 | cp/mL | 1.00E+07 | 1.00E+06 | |
| | BK Virus | N/A | cp/mL | 1.00E+07 | 1.00E+06 | |
| | Cytomegalovirus AD-169 | AD-169 | Cp/mL | 1.00E+07 | 1.00E+06 | |
| | Cell culture | | | | | |
| 2 | Candida albicans | CBS 562 | CFU/mL | 1.00E+07 | 1.00E+06 | 3/3 |
| | Chlamydia trachomatis | BOUR | IFU/mL | 1.00E+07 | 1.00E+06 | |
| | HIV-1 | B | cp/mL | 1.00E+07 | 1.00E+06 | |
| 3 | Dengue Virus Type 1 | Hawaii | TCID₅₀/mL | 5.00E+04 | 5.00E+03 | 3/3 |
| | Dengue Virus Type 2 | New Guinea | TCID₅₀/mL | 5.00E+04 | 5.00E+03 | |
| | | C | | | | |
| | Dengue Virus Type 3 | H87 | TCID₅₀/mL | 1.43E+06 | 1.43E+05 | |
| 4 | Herpes Simplex Virus Type 2 | MS | TCID₅₀/mL | 1.43E+05 | 1.43E+04 | 3/3 |
| | (HSV-2) | | | | | |
| | HIV Type 2 (HIV-2) | NIH-Z | TCID₅₀/mL | 1.00E+04 | 1.00E+03 | |
| | HPV type 18 purified plasmid | 18 | cp/mL | 1.00E+07 | 1.00E+06 | |
| | DNA | | | | | |
| 5 | Human Herpes Virus Type | GS | cp/mL | 1.00E+07 | 1.00E+06 | 3/3 |
| | 6A (HHV-6A) | | | | | |
| | Human Herpes Virus Type | Z29 | cp/mL | 1.00E+07 | 1.00E+06 | |
| | 6B (HHV-6B) | | | | | |
| | Human Herpes Virus Type 7 | SB | TCID₅₀/mL | 1.00E+06 | 1.00E+05 | |
| | (HHV-7) | | | | | |
| 6 | Human T-Lymphotropic | N/A | vp/mL | 1.00E+07 | 1.00E+06 | 3/3 |
| | Virus Type I (HTLV-I) | | | | | |
| | Human T-Lymphotropic | N/A | vp/mL | 1.00E+07 | 1.00E+06 | |
| | Virus Type II (HTLV-II) | | | | | |
| | Human Hepatitis B Virus | N/A | IU/mL | 1.79E+05 | 1.79E+04 | |
| | (HBV) | | | | | |
| 7 | Mycobacterium smegmatis | W-113 | CFU/mL | 1.00E+07 | 1.00E+06 | 3/3 |
| | Neisseria gonorrhoeae | NCTC 8375 | CFU/mL | 1.00E+07 | 1.00E+06 | |
| | Propionibacterium acnes | NCTC 737 | CFU/mL | 1.00E+07 | 1.00E+06 | |
| 8 | West Nile Virus (WNV) | NY 2001- | cp/mL | 1.00E+04 | 1.00E+03 | 3/3 |
| | | 6263 | | | | |
| | Vaccinia Virus Culture Fluid | “Vaccine | TCID₅₀/mL | 1.00E+07 | 1.00E+06 | |
| | | Strain” | | | | |
| | Trichomonas vaginalis | JH 31A #4 | cells/mL | 1.00E+06 | 1.00E+05 | |
| 9 | Staphylococcus epidermidis | RP62A | CFU/mL | 1.00E+07 | 1.00E+06 | 3/3 |
| | Human Parvovirus | B19 | IU/mL | 1.00E+07 | 1.00E+06 | |
| 10 | Hepatitis A virus | HM175 | IU/mL | 9.95E+06 | 9.95E+05 | 3/3 |
| | Dengue Virus Type 4 | H241 | TCID₅₀/mL | 5.00E+06 | 5.00E+05 | |
| | Synthetic Human | 16 | cp/mL | 1.00E+04 | 1.00E+03 | |
| | Papillomavirus 16 DNA | | | | | |
| 11 | Human Herpes Virus Type 8 | N/A | cp/mL | 1.00E+07 | 1.00E+06 | 3/3 |
| | (HHV-8) | | | | | |
| | Human Hepatitis C Virus | N/A | IU/mL | 2.63E+04 | 2.63E+03 | |
| | (HCV) | | | | | |
| | Staphylococcus aureus | NCTC 8532 | CFU/mL | 1.00E+07 | 1.00E+06 | |
| 12 | HSV-1 Strain | MacIntyre | TCID₅₀/mL | 1.00E+06 | 1.00E+05 | 3/3 |
| | Mycobacterium gordonae | L. Wayne | cp/mL | 1.00E+07 | 1.00E+06 | |
| | | W-1609 | | | | |

TABLE 25
HAdV Oligo Multiplex Performance Summary

| Panel | Avg Ct | SD Ct | Avg RFU | SD RFU | Avg T-Slope | SD T-Slope |
|---|---|---|---|---|---|---|
| 1 | 31.07 | 0.09 | 74469 | 2078 | 1528 | 60 |
| 2 | 30.53 | 0.34 | 76492 | 2863 | 1302 | 287 |
| 3 | 30.88 | 0.55 | 44127 | 764 | 1474 | 10 |
| 4 | 30.79 | 0.31 | 70886 | 6885 | 1286 | 98 |
| 5 | 30.40 | 0.09 | 64936 | 3775 | 1419 | 182 |
| 6 | 30.65 | 0.60 | 83775 | 1300 | 1474 | 69 |
| 7 | 31.18 | 0.24 | 82487 | 992 | 1317 | 125 |
| 8 | 30.98 | 0.14 | 78968 | 6541 | 1425 | 228 |
| 9 | 31.11 | 0.24 | 88952 | 3155 | 1482 | 58 |
| 10 | 30.76 | 0.13 | 56779 | 2483 | 1260 | 154 |
| 11 | 30.08 | 0.62 | 95074 | 2119 | 1325 | 92 |
| 12 | 29.60 | 0.13 | 63241 | 54693 | 864 | 662 |

### Example 13: Oligomers Containing Nucleotide Analogues

Exemplary HAdV-specific oligomers containing one or more nucleotide analogues are shown in Table 26 below.

TABLE 26

| SEQ | | | Corresponds |
|---|---|---|---|
| ID | | | to |
| NO | Sequence (5′ → 3′) | Description | SEQ ID NO* |
| 48 | AC^{5M}C^{5M}GTTAGTGAAAAC^{5M}GT | Amplification Oligomer | 4 |
| 49 | AC^{5M}C^{5M}ATC^{5M}AGTGAAAAC^{5M}GT | Amplification Oligomer | 5 |
| 50 | GC^{5M}CTTGTAGACGTAGGG | Amplification Oligomer | 6 |
| 51 | GC^{5M}C^{5M}TTGTAAAC^{5M}GTAGGG | Amplification Oligomer | 7 |
| 52 | GC^{5M}TTTGTAC^{5M}AC^{5M}GTAAGG | Amplification Oligomer | 8 |
| 53 | CC^{5M}TGC^{5M}TC^{5M}TC^{5M}ACAGATCA | Detection Probe | 15 |
| | CGGGAC | | |
| 54 | AC^{5M}C^{5M}GTC^{5M}AGTGAAAAC^{5M}GT | Amplification Oligomer | 3 |
| 55 | AC^{5M}C^{5M}GT^{dU}T^{dU}AGT^{dU}GAAAAC^{dC}GT | Amplification Oligomer | 4 |
| 56 | AC^{5M}C^{5M}AT^{dU}C^{5M}AGT^{dU}GAAAAC^{dC}GT | Amplification Oligomer | 5 |
| 57 | GC^{dC}C^{dC}T^{dU}T^{dU}GT^{dU}AGAC^{dU}GT^{dU}AGGG | Amplification Oligomer | 6 |
| 58 | GC^{dC}C^{dC}T^{dU}T^{dU}GT^{dU}AAAC^{dC}GT^{dU}AGGG | Amplification Oligomer | 7 |
| 59 | GC^{dC}T^{dU}T^{dU}T^{dU}GT^{dU}AC^{dC}AC^{dC}GT^{dU}AAGG | Amplification Oligomer | 8 |
| 60 | GC^{dC}T^{dU}T^{dU}T^{dU}GT^{dC}GGAC^{dU}GTAGGG | Amplification Oligomer | 9 |
| 61 | CC^{5M}TGC^{5M}TC^{5M}TC^{5M}AC^{5M}AGATC^{5M} | Detection Probe | 15 |
| | AC^{5M}GGGAC^{5M} | | |
| 62 | CCCGCCCTC^{5M}AC^{5M}AGATC^{5M}AC^{5M} | Detection Probe | 16 |
| | GGAAC | | |
| 63 | CCCGCTC^{5M}TC^{5M}AC^{5M}AGATC^{5M}AC^{5M} | Detection Probe | 17 |
| | GGGAC | | |
| 64 | C^{5M}AT^{dU}C^{5M}AC^{dU}GGT^{dU}T^{dU}T^{dU}C^{5M}GT^{dU} | Amplification Oligomer | 27 |
| | AAGAGG | | |
| 65 | TGGC^{5M}TTTTC^{5M}C^{5M}C^{5M}GAGGCACCACCA | Detection Probe | 29 |
| 66 | CCCGCC^{5M}C^{5M}TTAC^{5M}AGATC^{5M}AC^{5M}GGGAC^{5M} | Detection Probe | 30 |
| 67 | GTAATGGTC^{5M}AC^{5M}TC^{5M}GC^{5M}TGGACTC | Amplification Oligomer | 35 |
| 68 | CGGCGAC^{5M}C^{5M}TGAAAAC^{5M}C^{5M}ATC^{5M} | Detection Probe | 36 |
| | C^{5M}TGCATA | | |
| 69 | ATAC^{5M}GAGT^{5M}GGGT^{5M}GGAGTTTG | Detection Probe | 41 |
| | AGC^{5M}TGC | | |
| 70 | TTTAGC^{5M}C^{5M}T^{dU}AC^{5M}AAC^{5M}T^{dU}ATG | Amplification Oligomer | 42 |
| | GAGAC^{5M}C | | |
| 71 | TTTGGC^{5M}C^{5M}TAC^{5M}AACTACGGAGATG | Amplification Oligomer | 43 |

*“Corresponds to” means that the oligomer shown in the second column is an example of an oligomer containing one or more nucleotide analogues relative to this SEQ ID NO.

### Example 14

Modified HAdV-specific oligomers containing modifications to increase Tₘ relative to the primers and probes shown in Table 13 were prepared. The modified primers and probes are shown in Table 27 below.

TABLE 27

| | SEQ ID | |
|---|---|---|
| Oligo | NO | Sequence (5′ to 3′) |
| HAdV Penton FP 1B | 54 | AC^{5M}C^{5M}GTC^{5M}AGTGAAAAC^{5M}GT |
| HAdV Penton FP 2B | 55 | AC^{5M}C^{5M}GT^{dU}T^{dU}AGT^{dU}GAAAAC^{dC}GT |
| HAdV Penton FP 3B | 56 | AC^{5M}C^{5M}AT^{dU}C^{5M}AGT^{dU}GAAAAC^{dC}GT |
| HAdV Penton RP 1B | 57 | GC^{dC}C^{dC}T^{dU}T^{dU}GT^{dU}AGAC^{dC}GT^{dU}AGGG |
| HAdV Penton RP 2B | 58 | GC^{dC}C^{dC}T^{dU}T^{dU}GT^{dU}AAAC^{dC}GT^{dU}AGGG |
| HAdV Penton RP 3B | 59 | GC^{dC}T^{dU}T^{dU}T^{dU}GT^{dC}AC^{dC}AC^{dC}GT^{dU}AAGG |
| HAdV Penton RP 4B | 60 | GC^{dC}T^{dU}T^{dU}T^{dU}GT^{dU}GGAC^{dC}GT^{dU}AGGG |
| HAdV Penton PR 1B | 61 | Cal Fluor Orange-CC^{5M}TGC^{5M}TC^{5M}TC^{5M} |
| | | AC^{5M}AGATC^{5M}AC^{5M}GGGAC^{5M}-BHQ1 |
| HAdV Penton PR 2B | 62 | Cal Fluor Orange-CCCGCCCTC^{5M}AC^{5M} |
| | | AGATC^{5M}AC^{5M}GGAAC-BHQ1 |
| HAdV Penton PR 3B | 63 | Cal Fluor Orange-CCCGCTC^{5M}TC^{5M}AC^{5M}AGA |
| | | TC^{5M}AC^{5M}GGGAC-BHQ1 |

The modified primers and probes were tested penton gene plasmids for HAdV subtypes A-F. For human adenovirus serotypes representing each of subtypes A, B, C, D, E, and F, HAdV plasmid stock was diluted in STM (supplemented with 10,000 HeLa cells/mL) to provide HAdV plasmids at 50, 500, and 5,000 copies/reaction. Amplification and detection reactions were set up at 3 reactions per condition (3 reactions for each concentration of HAdV plasmid and 3 reactions negative for HAdV). The reactions were performed as real-time PCR reactions using a Panther Fusion system. Results are shown in Table 28 below.

TABLE 28

| | Conc. | Count of Ct/ | | |
|---|---|---|---|---|
| Subtype | (cp/rxn) | Count of sample | Avg Ct | Avg RFU |
| A | 5,000 | 3/3 | 32.71 | 16363.33 |
| | 500 | 3/3 | 36.16 | 8342.33 |
| | 50 | 2/3 | 40.03 | 2026.33 |
| B | 5,000 | 3/3 | 33.25 | 10442 |
| | 500 | 2/3 | 37.68 | 3482 |
| | 50 | 1/3 | 39.55 | 1248.33 |
| C | 5,000 | 3/3 | 27.95 | 17342 |
| | 500 | 3/3 | 31.28 | 15364.33 |
| | 50 | 3/3 | 35.18 | 9089 |
| D | 5,000 | 3/3 | 28.65 | 14870.67 |
| | 500 | 3/3 | 31.85 | 14723 |
| | 50 | 3/3 | 35.20 | 12088.33 |
| E | 5,000 | 3/3 | 27.90 | 20050.67 |
| | 500 | 3/3 | 31.25 | 16705.67 |
| | 50 | 3/3 | 34.97 | 8988.33 |
| F | 5,000 | 3/3 | 26.75 | 23841 |
| | 500 | 3/3 | 30.38 | 17360.67 |
| | 50 | 3/3 | 33.81 | 11586 |

HAdV subtypes A and B had the latest Cts and lowest RFU values. HAdV subtypes E and F had the earliest Cts and highest RFU values. Overall, the primers of probes shown Table 13 have better Cts; however, the modified primers and probes of Table 27 have better t-slopes and standard slopes with the exception of subtype A. The raw curve of an internal control reaction of HAdV subtypes A-F showed significant background interference and confirmed plasmids caused interference due to their degradation. Further testing is suggested to determine if the modified oligomers of Table 27 perform better than those of Table 13.

### Example 15

The modified primers and probes of Table 27 were tested on live HAdV viruses for subtypes A-F. This study compared the performance of the modified primers and probes against that of the oligomers of Table 13 (“nominal” primers and probes).

The assay was run as a real-time PCR reaction using the Panther Fusion system. Virus was spiked into STM containing 10,000 HeLa cells/mL in a 10-fold dilution series of three concentrations: 1E3 TCID50/mL. 1E2 TCID50/mL, and 1E1 TCID50/mL. Amplification and detection reactions were set up at 3 reactions per condition. The reactions were performed as real-time PCR reactions using a Panther Fusion system. Results are shown in Table 29 below.

TABLE 29

| | Copies/rxn | | | |
|---|---|---|---|---|
| Subtype | (TCID₅₀/mL) | PPR* | Avg Ct | Avg RFU |
| A | 1E3 | Modified | 19.20 | 27827.67 |
| | | Nominal | 24.36 | 19439.33 |
| | 1E2 | Modified | 22.37 | 26485.33 |
| | | Nominal | 27.59 | 16647 |
| | 1E1 | Modified | 25.55 | 22855.33 |
| | | Nominal | 31.16 | 12084.67 |
| B | 1E3 | Modified | 22.97 | 22972 |
| | | Nominal | 24.55 | 16511.33 |
| | 1E2 | Modified | 27.00 | 21347.33 |
| | | Nominal | 26.76 | 13774.33 |
| | 1E1 | Modified | 33.12 | 13932 |
| | | Nominal | 32.15 | 11415.33 |
| C | 1E3 | Modified | 30.03 | 16841.33 |
| | | Nominal | 28.20 | 12979.33 |
| | 1E2 | Modified | 33.05 | 14415.67 |
| | | Nominal | 33.38 | 11510 |
| | 1E1 | Modified | 35.20 | 9898.67 |
| | | Nominal | 35.66 | 7575.67 |
| D | 1E3 | Modified | 22.02 | 21671 |
| | | Nominal | 21.39 | 19051 |
| | 1E2 | Modified | 26.13 | 19198.33 |
| | | Nominal | 26.55 | 15480 |
| | 1E1 | Modified | 29.04 | 20099 |
| | | Nominal | 29.72 | 9586.67 |
| E | 1E3 | Modified | 18.69 | 25503 |
| | | Nominal | 17.65 | 18211.67 |
| | 1E2 | Modified | 21.95 | 25388.33 |
| | | Nominal | 21.52 | 19138.67 |
| | 1E1 | Modified | 24.35 | 23473.33 |
| | | Nominal | 24.61 | 18573.33 |
| F | 1E3 | Modified | 20.95 | 31494.67 |
| | | Nominal | 21.21 | 26650.33 |
| | 1E2 | Modified | 24.43 | 24919 |
| | | Nominal | 24.51 | 22217 |
| | 1E1 | Modified | 27.88 | 23098.33 |
| | | Nominal | 27.95 | 19770 |

*PPR = Primer and probe set; “modified” PPR is shown in Table 27 and “nominal” PPR is shown in Table 13.

Significant Ct improvement was observed for HAdV subtype A (˜5 Cts). The modified primers and probes also had a significant improvement in amplification compared to the nominal primers and probes for all subtypes. The modified primers and probes also observed better linearization for subtypes A, C, E, and F. Further experimentation is suggested to determine a preliminary LoD in STM+HeLa cells for subtypes A-F using the modified primers and probe.

### Example 16

Preliminary limits of detection (LoDs) of HAdV subtypes A-F were determined using the modified primers and probes shown in Table 27. The assay was run as a real-time PCR reaction using the Panther Fusion system. Virus was spiked into STM containing 10,000 HeLa cells/mL in a 10-fold dilution series. Six concentrations were used ranging from 1E3 TCID50/mL to 1E-3 TCID50/mL. Results are shown in Table 30 below.

TABLE 30

| | Conc. | Count of Ct/ | | |
|---|---|---|---|---|
| Subtype | (TCID₅₀/mL) | Count of sample | Avg Ct | Avg RFU |
| A | 1E1 | 3/3 | 25.90 | 21359 |
| | 1E0 | 3/3 | 29.01 | 18591 |
| | 1E-1 | 3/3 | 32.48 | 14013.67 |
| | 1E-2 | 3/3 | 36.02 | 8497.67 |
| | 1E-3 | 3/3 | 39.10 | 4140.67 |
| B | 1E1 | 3/3 | 34.11 | 11893.33 |
| | 1E0 | 3/3 | 37.20 | 7134 |
| | 1E-1 | 0/3 | — | 126 |
| | 1E-2 | 0/3 | — | 123 |
| | 1E-3 | 0/3 | — | 124.33 |
| C | 1E1 | 3/3 | 37.77 | 6173.67 |
| | 1E0 | 0/3 | — | 133.67 |
| | 1E-1 | 0/3 | — | 128.67 |
| | 1E-2 | 0/3 | — | 118 |
| | 1E-3 | 0/3 | — | 134.33 |
| D | 1E1 | 3/3 | 29.53 | 17763.33 |
| | 1E0 | 3/3 | 33.07 | 13088.67 |
| | 1E-1 | 3/3 | 36.14 | 9173.33 |
| | 1E-2 | 2/3 | 38.66 | 3291.67 |
| | 1E-3 | 0/3 | — | 119.67 |
| E | 1E1 | 3/3 | 26.20 | 20188 |
| | 1E0 | 3/3 | 29.57 | 19067 |
| | 1E-1 | 3/3 | 32.91 | 15260.67 |
| | 1E-2 | 3/3 | 36.11 | 9665 |
| | 1E-3 | 1/3 | 39.43 | 1417.67 |
| F | 1E1 | 3/3 | 28.07 | 21782.33 |
| | 1E0 | 3/3 | 31.18 | 18160 |
| | 1E-1 | 3/3 | 34.48 | 11558.33 |
| | 1E-2 | 1/3 | 36.89 | 2394.67 |
| | 1E-3 | 0/3 | — | 98 |

Preliminary LoDs (TCID50/mL) were determined and are shown in Table 31 below.

TABLE 31

| Subtype | LoD (TCID₅₀/mL) |
|---|---|
| A | 1E-3 |
| B | 1E0 |
| C | 1E1 |
| D | 1E-1 |
| E | 1E-2 |
| F | 1E-1 |

The modified HAdV penton gene multiplex had the most sensitivity for HAdV A, followed by HAdV E. Subtypes A, D, E, and F had good slope values and were approximately 0.1-0.2 away from the ideal slope value of −3.2. Further determination of LoD's will be performed and determined by testing for concentrations ½ log above and below the preliminary LoDs for all subtypes.

### Example 17

Limits of detection (LoDs) of HAdV subtypes A-F were determined using plasma and the modified primers and probes shown in Table 27. Virus was spiked into pooled plasma at the concentrations shown in Table 32 below. The assay was run as a real-time PCR reaction using the Panther Fusion system. Results are shown in Table 33 below.

TABLE 32

| HAdV Subtype | Sample Conc. (TCID₅₀/mL) |
|---|---|
| A | 3E-3 |
| | 1E-3 |
| | 3E-4 |
| B | 3E0 |
| | 1E0 |
| | 3E-1 |
| C | 3E1 |
| | 1E1 |
| | 3E0 |
| D | 3E-1 |
| | 1E-1 |
| | 3E-2 |
| E | 3E-2 |
| | 1E-2 |
| | 3E-3 |
| F | 3E-1 |
| | 1E-1 |
| | 3E-2 |

TABLE 33

| | Count of Ct/ | | |
|---|---|---|---|
| Sample | Count of sample | Avg Ct | Avg RFU |
| Positive Control | 4/4 | 26.48 | 25637.25 |
| Negative Control | 0/4 | — | 120.25 |
| HAdV A 3E-3 | 4/4 | 37.28 | 6765.25 |
| HAdV A 1E-3 | 2/4 | 39.37 | 2058 |
| HAdV A 3E-4 | 1/4 | 41 | 666.25 |
| HAdV B 3E0 | 4/4 | 32.53 | 14754.5 |
| HAdV B 1E0 | 4/4 | 33.28 | 13194.5 |
| HAdV B 3E-1 | 4/4 | 35.27 | 10212.5 |
| HAdV C 3E1 | 4/4 | 34.10 | 13274.5 |
| HAdV C 1E1 | 4/4 | 35.35 | 10939 |
| HAdV C 3E0 | 4/4 | 37.19 | 7732.25 |
| HAdV D 3E-1 | 4/4 | 33.25 | 12267 |
| HAdV D 1E-1 | 4/4 | 35.19 | 9949 |
| HAdV D 3E-2 | 4/4 | 36.07 | 8549.25 |
| HAdV E 3E-2 | 4/4 | 32.97 | 13015.75 |
| HAdV E 1E-2 | 4/4 | 34.49 | 10973.75 |
| HAdV E 3E-3 | 4/4 | 35.67 | 10124 |
| HAdV F 3E-1 | 4/4 | 32.77 | 16066.75 |
| HAdV F 1E-1 | 4/4 | 34.38 | 13762.75 |
| HAdV F 3E-2 | 4/4 | 36.31 | 9231.5 |

All subtypes were detected at ½ log above and ½ log below the LoDs which were predetermined in the experiment described in Example 16, with the exception of subtype A. Subtype A had detection up to 3E-3.

### Example 18

Four new primer/probe designs were screened to determine if these primer/probe sets can improve detection of HAdV subtypes A, C, E, and F when compared to the primer and probes of Table 27. The new primer/probe sets are shown in Table 34 below.

TABLE 34

| Tar- | Olig- | | |
|---|---|---|---|
| get | omer | | |
| (HAdV | (SEQ | | |
| sub- | ID | | |
| type) | NO) | Sequence (5′ to 3′) | Description |
| A | 26 | CGGTGGAACTGCAGACAG | Forward primer |
| | 27 | C^{5M}AT^{dU}C^{5M}AC^{5M}GGT^{dU}T^{dU} | Reverse primer |
| | | T^{dU}C^{5M}GT^{dU}AAGAGG | |
| | 28 | CGCTGAACTCCACTGAT | Reverse primer |
| | | ACTGCT | |
| | 29 | CalRed-TGGC^{5M}TTTTC^{5M} | Detection Probe |
| | | C^{5M}C^{5M}GAGGCACCACC | |
| | | A-BHQ2 | |
| | 30 | CalRed-CCCGCC^{5M}C^{5M} | Detection Probe |
| | | TTAC^{5M}AGATC^{5M}AC^{5M}GG | |
| | | GAC^{5M}-BHQ2 | |
| C | 33 | ACAGACCATCAATCTTG | Forward primer |
| | | ACGAC | |
| | 34 | ACCATCACCCGCGCCTT | Reverse primer |
| | | AAAC | |
| | 35 | GTAATGGTC^{5M}AC^{5M}TC^{5M}G | Reverse primer |
| | | C^{5M}TGGACTC | |
| | 36 | Q760-CGGCGAC^{5M}C^{5M}T | Detection probe |
| | | GAAAAC^{5M}C^{5M}ATC^{5M}C^{5M} | |
| | | TGCATA-BHQ2 | |
| E | 38 | GATGGTAGTCAGGATGAG | Forward primer |
| | | CTG | |
| | 39 | AATGGTCATGGTCACCG | Reverse primer |
| | | AGAAGT | |
| | 40 | TAACGGTCACGCGCTG | Reverse primer |
| | | GAC | |
| | 41 | Q670-ATAC^{5M}GAGTUGGG | Detection probe |
| | | T^{dU}UGGAGTTTGAGC^{5M}TG | |
| | | C-BHQ2 | |
| F | 42 | TTTAGC^{5M}C^{5M}T^{dU}AC^{5M}AA | Forward primer |
| | | C^{5M}T^{dU}ATGGAGAC^{5M}C | |
| | 43 | TTTGGC^{5M}C^{5M}TAC^{5M}AAC | Forward primer |
| | | TACGGAGATG | |
| | 44 | TC^{5M}TTGC^{5M}ATC^{5M}ATGT | Reverse primer |
| | | C^{5M}GGG | |
| | 45 | AC^{5M}TC^{5M}C^{5M}AC^{5M}T^{dU}GA | Reverse primer |
| | | T^{dU}AC^{5M}TGC^{5M}TGC | |
| | 46 | FAM-ACCAGTAC^{5M}AC^{5M} | Detection probe |
| | | C^{5M}TGC^{5M}TGCGACC | |
| | | C-BHQ2 | |

For each of human adenovirus serotypes 31, 2, 4, and 41 (representing subtypes A, C, E, and F, respectively), virus was spiked into STM, or STM containing 10,000 HeLa cells/mL, at concentrations of 1E2 TCID50/mL and 1E1 TCID50/mL. Negative controls (STM without adenovirus) were prepared with and without HeLa cells. Each new primer and probe set shown in Table 34 and the modified primer and probe set of Table 27 were tested. The assay was run as a real-time PCR reaction using the Panther Fusion system. Results are shown in Table 35 below.

TABLE 35

| HAdV | HeLa/ | Sample conc. | | Avg | Avg |
|---|---|---|---|---|---|
| Subtype | No HeLa | (TCID₅₀/mL) | PPR* | Ct | RFU |
| A | HeLa | 1E1 | Modified | 26.29 | 22567.5 |
| | | | New HAdV A | 25.47 | 7797.5 |
| | | 1E2 | Modified | 22.66 | 22679.5 |
| | | | New HAdV A | 22.01 | 7530 |
| | No HeLa | 1E1 | Modified | 26.11 | 23058 |
| | | | New HAdV A | 25.39 | 7402.5 |
| | | 1E2 | Modified | 22.61 | 28668.5 |
| | | | New HAdV A | 22.16 | 7540.5 |
| C | HeLa | 1E1 | Modified | 37.94 | 7282 |
| | | | New HAdV C | 38.23 | 973.5 |
| | | 1E2 | Modified | 33.46 | 15173 |
| | | | New HAdV C | 34.36 | 1131.5 |
| | No HeLa | 1E1 | Modified | 37.16 | 8897.5 |
| | | | New HAdV C | 37.58 | 1063.5 |
| | | 1E2 | Modified | 34.09 | 15157.5 |
| | | | New HAdV C | 34.86 | 1104.5 |
| E | HeLa | 1E1 | Modified | 27.16 | 18645 |
| | | | New HAdV E | 28.87 | 779 |
| | | 1E2 | Modified | 21.50 | 22912 |
| | | | New HAdV E | 23.12 | 878.5 |
| | No HeLa | 1E1 | Modified | 26.65 | 20491.5 |
| | | | New HAdV E | 28.68 | 823.5 |
| | | 1E2 | Modified | 23.545 | 21758.5 |
| | | | New HAdV E | 25.59 | 821 |
| F | HeLa | 1E1 | Modified | 28.04 | 22924.5 |
| | | | New HAdV F | 31.46 | 16241.5 |
| | | 1E2 | Modified | 24.82 | 26908 |
| | | | New HAdV F | 28.04 | 17073.5 |
| | No HeLa | 1E1 | Modified | 27.67 | 24430 |
| | | | New HAdV F | 31.22 | 15320.5 |
| | | 1E2 | Modified | 24.29 | 30104 |
| | | | New HAdV F | 27.63 | 16646.5 |

*PPR = Primer and probe set; “modified” PPR is shown in Table 27; “new” PPR for each of HAdV subtypes A, C, E, and F is shown in Table 34.

Both the new HAdV A primers and probe and the modified primers and probes of Table 27 had similar Cts, but the modified primers and probes had much better RFU as well as steeper T-dopes. For each of HAdV C, E, and F, the primers and probes of Table 27 had earlier Cts as well as better T-slopes and RFUs than the new HAdV C, E, and F primers and probes, respectively.

### SEQUENCES

TABLE 36

| SEQ | | |
|---|---|---|
| ID NO | Sequence (5′ → 3′) | Description |
| 3 | ACCGTCAGTGAAAACGT | Amplification Oligomer |
| 4 | ACCGTTAGTGAAAACGT | Amplification Oligomer |
| 5 | ACCATCAGTGAAAACGT | Amplification Oligomer |
| 6 | GCCTTGTAGACGTAGGG | Amplification Oligomer |
| 7 | GCCTTGTAAACGTAGGG | Amplification Oligomer |
| 8 | GCTTTGTACACGTAAGG | Amplification Oligomer |
| 9 | GCTTTGTGGACGTAGGG | Amplification Oligomer |
| 10 | TCACTCGCTGGACTC | Amplification Oligomer |
| 11 | TCACGCGCTGGACTC | Amplification Oligomer |
| 12 | TCACACGTTGGACTC | Amplification Oligomer |
| 13 | TCACGCGCTGAACTC | Amplification Oligomer |
| 15 | CCTGCTCTCACAGATCACGGGAC | Detection Probe |
| 16 | CCCGCCCTCACAGATCACGGAAC | Detection Probe |
| 17 | CCCGCTCTCACAGATCACGGGAC | Detection Probe |
| 18 | ACCRTYAGTGAAAACGT | Amplification Oligomer |
| 19 | CCYGCYCTCACAGATCACGGRAC | Detection Probe |
| 20 | TCACDCGYTGRACTC | Amplification Oligomer |
| 21 | GCYTTGTRVACGTARGG | Amplification Oligomer |
| 22 | ACCRTYAGTGAAAACGTTCCYGCYCTYACAGATCACGGRACCCTGCCGCTG | Amplicon |
| | CGTAGCAGTATCAGTGGAGTRCAYCGDGTCACCATCACTGACGCCCGCCGC | |
| | CGGACCTGTCCRTACGTVRACAAYGC | |
| 23 | CCYGCYCTTACAGATCACGGRACCCTGCCGCTGCGTAGCAGTATCAGTGGA | Amplicon |
| | GTRCAYCGDGTCACCATCACTGACGCCCGCCGCCGGACCTGTCCRTACGTV | |
| | RACAAYGC | |
| 24 | ACCRTYAGTGAAAACGTTCCYGCYCTYACAGATCACGGRACCCTGCCGCTG | Amplicon |
| | CGTAGCAGTATCAGTGGAGTRCAYCGDGTCA | |
| 25 | GenBank Accession No. X73487.1 | Human Adenovirus |
| | | Reference Sequence |
| 26 | CGGTGGAACTGCAGACAG | Amplification Oligomer |
| 27 | CATCACGGTTTCGTAAGAGG | Amplification Oligomer |
| 28 | CGCTGAACTCCACTGATACTGCT | Amplification Oligomer |
| 29 | TGGCTTTTCCCGAGGCACCACC | Detection Probe |
| 30 | CCCGCCCTTACAGATCACGGGAC | Detection Probe |
| 31 | TGGACTCCCCGGATACTGCT | Amplification Oligomer |
| 32 | TGCCGTTGCGCAGCAGTATC | Amplification Oligomer |
| 33 | ACAGACCATCAATCTTGACGAC | Amplification Oligomer |
| 34 | ACCATCACCCGCGCCTTAAAC | Amplification Oligomer |
| 35 | GTAATGGTCACTCGCTGGACTC | Amplification Oligomer |
| 36 | CGGCGACCTGAAAACCATCCTGCATA | Detection Probe |
| 37 | ACGGTCACTCGCTGGACTC | Amplification Oligomer |
| 38 | GATGGTAGTCAGGATGAGCTG | Amplification Oligomer |
| 39 | AATGGTCATGGTCACCGAGAAGT | Amplification Oligomer |
| 40 | TAACGGTCACGCGCTGGAC | Amplification Oligomer |
| 41 | ATACGAGTGGGTGGAGTTTGAGCTGC | Detection Probe |
| 42 | TTTAGCCTACAACTATGGAGACC | Amplification Oligomer |
| 43 | TTTGGCCTACAACTACGGAGATG | Amplification Oligomer |
| 44 | TCTTGCATCATGTCGGG | Amplification Oligomer |
| 45 | ACTCCACTGATACTGCTGC | Amplification Oligomer |
| 46 | ACCAGTACACCTGCTGCGACCC | Detection Probe |
| 47 | CCYGCYCTYACAGATCACGGRAC | Detection Probe |

From the foregoing, it will be appreciated that, although specific embodiments have been described herein for purposes of illustration, various modifications may be made without deviating from the spirit and scope of the disclosure. All publications, patents, and patent applications cited herein are hereby incorporated by reference in their entireties for all purposes.

1. A composition or kit for determining the presence or absence of human adenovirus in a sample, said composition or kit comprising:
at least two amplification oligomers capable of amplifying a target region of a human adenovirus target nucleic acid, wherein
(a) at least one amplification oligomer comprises a target-hybridizing sequence as shown in SEQ ID NO: 18, or an RNA equivalent or a DNA/RNA chimeric thereof and including from 0 to 9 nucleotide analogs; and
(b) at least one amplification oligomer is selected from the group consisting of
(i) an oligomer comprising a target-hybridizing sequence as shown in SEQ ID NO:21, or an RNA equivalent or a DNA/RNA chimeric thereof and including from 0 to 9 nucleotide analogs, and
(ii) an oligomer comprising a target-hybridizing sequence as shown in SEQ ID NO:20, or an RNA equivalent or a DNA/RNA chimeric thereof and including from 0 to 9 nucleotide analogs.

2. The composition or kit of claim 1, further comprising at least one detection probe oligomer comprising a target-hybridizing sequence as shown in SEQ ID NO:19, or an RNA equivalent or a DNA/RNA chimeric thereof and including from 0 to 9 nucleotide analogs.

3. (canceled)

4. A formulation for amplifying a target region of a human adenovirus target nucleic acid in a sample, the formulation comprising:
(a) at least one amplification oligomer comprising a target-hybridizing sequence as shown in SEQ ID NO:18, or an RNA equivalent or a DNA/RNA chimeric thereof and including from 0 to 9 nucleotide analogs;
(b) at least one amplification oligomer selected from the group consisting of (i) an oligomer comprising a target-hybridizing sequence as shown in SEQ ID NO:21, or an RNA equivalent or a DNA/RNA chimeric thereof and including from 0 to 9 nucleotide analogs, and (ii) an oligomer comprising a target-hybridizing sequence as shown in SEQ ID NO:20, or an RNA equivalent or a DNA/RNA chimeric thereof and including from 0 to 9 nucleotide analogs; and
(c) a buffer.

5. The formulation of claim 4, wherein the formulation is a dried composition.

6-7. (canceled)

8. A kit comprising a dried composition as in claim 5.

9. The kit of claim 8, further comprising a reconstitution reagent, wherein the dried composition is in a first vial within the kit and the reconstitution reagent is in a second vial within the kit.

10. A method for preparing an aqueous reaction mixture for determining the presence or absence of a human adenovirus in a sample, the method comprising the step of combining a dried composition as described in claim 5 with a reconstitution reagent to make an aqueous reaction mixture.

11. A method for determining the presence or absence of human adenovirus in a sample, the method comprising:
(1) contacting a sample, said sample suspected of containing human adenovirus, with at least two oligomers capable of amplifying a target region of a human adenovirus target nucleic acid, said oligomer combination comprising
(a) at least one amplification oligomer comprising a target-hybridizing sequence as shown in SEQ ID NO: 18, or an RNA equivalent or a DNA/RNA chimeric thereof and including from 0 to 9 nucleotide analogs; and
(b) at least one amplification oligomer selected from the group consisting of
(i) an oligomer comprising a target-hybridizing sequence as shown in SEQ ID NO:21, or an RNA equivalent or a DNA/RNA chimeric thereof and including from 0 to 9 nucleotide analogs, and
(ii) an oligomer comprising a target-hybridizing sequence as shown in SEQ ID NO:20, or an RNA equivalent or a DNA/RNA chimeric thereof and including from 0 to 9 nucleotide analogs;
(2) performing an in vitro nucleic acid amplification reaction, wherein any human adenovirus target nucleic acid present in the sample is used as a template for generating an amplification product; and
(3) detecting the presence or absence of the amplification product, thereby determining the presence or absence of human adenovirus in the sample.

12. The method of claim 11, wherein the detecting step (3) comprises contacting the in vitro nucleic acid amplification reaction with at least one detection probe oligomer configured to specifically hybridize to the amplification product under conditions whereby the presence or absence of the amplification product is determined, thereby determining the presence or absence of human adenovirus in the sample.

13. The method of claim 12, wherein the at least one detection probe oligomer comprises a target-hybridizing sequence as shown in SEQ ID NO: 19, or an RNA equivalent or a DNA/RNA chimeric thereof and including from 0 to 9 nucleotide analogs.

14-23. (canceled)

24. An oligonucleotide comprising a human adenovirus (HAdV)-specific target-hybridizing sequence as shown in SEQ ID NO:18, SEQ ID NO:21, SEQ ID NO:20, SEQ ID NO: 19, SEQ ID NO:26, SEQ ID NO:27, SEQ ID NO:28, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, or SEQ ID NO:46, or SEQ ID NO:47, or an RNA equivalent or a DNA/RNA chimeric thereof and including from 0 to 9 nucleotide analogs, wherein the 3′ end of said oligonucleotide is attached to a solid support.

25. The oligonucleotide of claim 24, wherein the HAdV-specific target-hybridizing sequence is SEQ ID NO: 18, SEQ ID NO:20, SEQ ID NO:21, or SEQ ID NO:47, or an RNA equivalent or a DNA/RNA chimeric thereof and including from 0 to 9 nucleotide analogs.

26. The oligonucleotide of claim 25, wherein the HAdV-specific target-hybridizing sequence is SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, SEQ ID NO:13, SEQ ID NO: 15, SEQ ID NO: 16, or SEQ ID NO: 17, or SEQ ID NO:30, or an RNA equivalent or a DNA/RNA chimeric thereof and including from 0 to 9 nucleotide analogs.

27. The composition or kit of claim 1, wherein the target-hybridizing sequence of (a) is SEQ ID NO:3, SEQ ID NO:4, or SEQ ID NO:5, or an RNA equivalent or a DNA/RNA equivalent thereof and including from 0 to 9 nucleotide analogs.

28. The composition or kit of claim 1, wherein the composition or kit comprises at least two amplification oligomers as in (a)
optionally wherein the composition or kit comprises first, second, and third amplification oligomers as in (a), wherein
the first amplification oligomer as in (a) comprises a target-hybridizing sequence as shown in SEQ ID NO:3, or an RNA equivalent or a DNA/RNA chimeric thereof and including from 0 to 9 nucleotide analogs,
the second amplification oligomer as in (a) comprises a target-hybridizing sequence as shown in SEQ ID NO:4, or an RNA equivalent or a DNA/RNA chimeric thereof and including from 0 to 9 nucleotide analogs, and
the third amplification oligomer as in (a) comprises a target-hybridizing sequence as shown in SEQ ID NO:5, or an RNA equivalent or a DNA/RNA chimeric thereof and including from 0 to 9 nucleotide analogs.

29. The composition or kit of claim 1, wherein the composition or kit comprises the at least one amplification oligomer of (b)(i), optionally wherein the target-hybridizing sequence of (b)(i) is SEQ ID NO:6, SEQ ID NO:7, SEQ ID NO:8, or SEQ ID NO:9, or an RNA equivalent or a DNA/RNA equivalent thereof and including from 0 to 9 nucleotide analogs.

30. The composition or kit of claim 1, wherein the composition or kit comprises at least two amplification oligomers as in (b)(i),
optionally wherein the composition or kit comprises first, second, third, and fourth amplification oligomers as in (b)(i), wherein
the first amplification oligomer as in (b)(i) comprises a target-hybridizing sequence as shown in SEQ ID NO:6, or an RNA equivalent or a DNA/RNA chimeric thereof and including from 0 to 9 nucleotide analogs,
the second amplification oligomer as in (b)(i) comprises a target-hybridizing sequence as shown in SEQ ID NO:7, or an RNA equivalent or a DNA/RNA chimeric thereof and including from 0 to 9 nucleotide analogs,
the third amplification oligomer as in (b)(i) comprises a target-hybridizing sequence as shown in SEQ ID NO:8, or an RNA equivalent or a DNA/RNA chimeric thereof and including from 0 to 9 nucleotide analogs, and
the fourth amplification oligomer as in (b)(i) comprises a target-hybridizing sequence as shown in SEQ ID NO:9, or an RNA equivalent or a DNA/RNA chimeric thereof and including from 0 to 9 nucleotide analogs.

31. The composition or kit of claim 1, wherein the composition or kit comprises the at least one amplification oligomer of (b)(ii), optionally wherein the target-hybridizing sequence of (b)(ii) is SEQ ID NO: 10, SEQ ID NO:11, SEQ ID NO:12, or SEQ ID NO:13, or an RNA equivalent or a DNA/RNA equivalent thereof and including from 0 to 9 nucleotide analogs.

32. The composition or kit of claim 1, wherein the composition or kit comprises at least two amplification oligomers as in (b)(ii),
optionally wherein the composition or kit comprises first, second, third, and fourth amplification oligomers as in (b)(ii), wherein
the first amplification oligomer as in (b)(ii) comprises a target-hybridizing sequence as shown in SEQ ID NO: 10, or an RNA equivalent or a DNA/RNA chimeric thereof and including from 0 to 9 nucleotide analogs,
the second amplification oligomer as in (b)(ii) comprises a target-hybridizing sequence as shown in SEQ ID NO:11, or an RNA equivalent or a DNA/RNA chimeric thereof and including from 0 to 9 nucleotide analogs,
the third amplification oligomer as in (b)(ii) comprises a target-hybridizing sequence as shown in SEQ ID NO:12, or an RNA equivalent or a DNA/RNA chimeric thereof and including from 0 to 9 nucleotide analogs, and
the fourth amplification oligomer as in (b)(ii) comprises a target-hybridizing sequence as shown in SEQ ID NO: 13, or an RNA equivalent or a DNA/RNA chimeric thereof and including from 0 to 9 nucleotide analogs.

33. The composition or kit of claim 2, wherein the at least one detection probe oligomer target-hybridizing sequence is SEQ ID NO:15, SEQ ID NO:16, or SEQ ID NO:17, or an RNA equivalent or a DNA/RNA equivalent thereof and including from 0 to 9 nucleotide analogs.
